(19) United States
(12) Patent Application Publication
(10) Pub. No.: US 2013/0245385 A1
(43) Pub. Date: Sep. 19, 2013

(54) SELECTIVE ANTI-HLA ANTIBODY REMOVAL DEVICE AND METHODS OF PRODUCTION AND USE THEREOF

(71) Applicants: William H. Hildebrand, Edmond, OK (US); Curtis McMurtrey, Oklahoma City, OK (US); Rico Buchli, Edmond, OK (US); Steven Cate, Moore, OK (US)

(72) Inventors: William H. Hildebrand, Edmond, OK (US); Curtis McMurtrey, Oklahoma City, OK (US); Rico Buchli, Edmond, OK (US); Steven Cate, Moore, OK (US)

(73) Assignee: THE BOARD OF REGENTS OF THE UNIVERSITY OF OKLAHOMA, Norman, OK (US)

(21) Appl. No.: 13/860,897

(22) Filed: Apr. 11, 2013

Related U.S. Application Data
(63) Continuation-in-part of application No. 13/460,433, filed on Apr. 30, 2012.
Application No. 13/460,433 is a continuation-in-part of application No. 12/859,002, filed on Aug. 18, 2010.
(60) Provisional application No. 61/622,607, filed on Apr. 11, 2012.
(60) Provisional application No. 61/480,865, filed on Apr. 29, 2011.
(60) Provisional application No. 61/234,937, filed on Aug. 18, 2009.
(60) Provisional application No. 61/333,827, filed on May 12, 2010.

(51) Int. Cl. C07K 16/28; B01D 15/00

(52) U.S. Cl. CPC C07K 16/28; B01D 15/00
USPC 600/300; 530/389.6; 210/483

(57) ABSTRACT

An anti-MHC removal device includes a serologically active, soluble MHC moiety covalently coupled to a solid support. Methods of production include covalently coupling the serologically active, soluble MHC moiety to the solid support. Methods of use of the anti-MHC removal device include contacting a biological sample with the device so that antibodies specific for the MHC moiety are removed from the biological sample.

### CROSS REFERENCE TO RELATED APPLICATIONS

This application claims benefit under 35 USC 119(e) of U.S. provisional application Ser. No. 61/622,607, filed Apr. 11, 2012. This application is also a continuation-in-part of U.S. Ser. No. 13/460,433, filed Apr. 30, 2012; which claims benefit under 35 USC 119(e) of U.S. provisional application Ser. No. 61/480,865, filed Apr. 29, 2011.

The '433 application is also a continuation-in-part of U.S. Ser. No. 12/859,002, filed Aug. 18, 2010; which claims benefit under 35 USC 119(e) of U.S. provisional application Ser. No. 61/234,937, filed Aug. 18, 2009; and Ser. No. 61/333,827, filed May 12, 2010.

The entire contents of each of the above-referenced patents and patent applications are hereby expressly incorporated herein by reference.

### STATEMENT REGARDING FEDERALLY SPONSORED RESEARCH OR DEVELOPMENT

Not Applicable.

### BACKGROUND OF THE INVENTIVE CONCEPT(S)

1. Field of the Invention

The presently disclosed and claimed inventive concept(s) relates generally to a methodology of removing anti-HLA antibodies from a sample, as well as a device utilized therefor.

2. Description of the Background Art

Human cells express on their surface an incredibly large number of membrane-bound proteins, all of which display individual properties and physiological functions. From this large array of surface cell proteins, a number of clinical procedures require characterization of the human major histocompatibility complex (MHC) class I and II membrane-bound molecules. The human MHC class I and class II molecules are known as human leukocyte antigens, or HLA. The HLA class I and class II molecules are responsible for presenting peptide antigens to receptors located on the surface of T-lymphocytes, Natural Killer Cells (NK), and possibly other immune effector and regulatory cells. Display of peptide antigens on the MHC I and MHC II molecules are the basis for the recognition of “self vs. non-self” and the onset of important immune responses such as transplant rejection, graft-versus-host-disease, autoimmune disease, and healthy anti-viral and anti-bacterial immune responses.

HLA class I and class II molecules differ from person to person. Each person expresses a different complement of class I and class II on the surface of their cells. For transplant purposes it is important to determine which of the multiple HLA expressed on a cell are recognized by the antibodies of another individual. The presence of anti-HLA antibodies in a transplant recipient can lead to hyperacute organ rejection. It is often difficult to determine which of many HLA are recognized by antibodies because sera can have antibodies to non-HLA proteins and multiple HLA molecules, and sera may cross-react among different HLA molecules. With many human proteins, many HLA proteins, antibodies to multiple human proteins, and antibodies cross-reactive to various HLA proteins, it can be difficult when screening patients for organ transplantation to ascertain which of the many HLA in the population, and expressed on an organ to be transplanted, are recognized by antibodies. Antibodies to HLA proteins may also lead to problems during the transfusion of blood products, whereby antibodies in the blood of the blood donor may react with the HLA class I and class II antigens of the recipient of the blood product. Antibodies in the blood product that recognize the recipient's HLA may lead to transfusion related acute lung injury (TRAM.

Class I MHC molecules, designated HLA class I in humans, bind and display peptide antigen ligands upon the cell surface. The peptide antigen ligands presented by the class I MHC molecule are derived from either normal endogenous proteins (“self”) or foreign proteins (“nonself”) introduced into the cell. Nonself proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptide antigens bound and presented by HLA class II are derived from extracellular foreign antigens, such as products of bacteria that multiply outside of cells, wherein such products include protein toxins secreted by the bacteria or any other bacterial protein to which the human immune system might respond in a protective manner. In this manner, class II molecules convey information regarding the existence of pathogens in extracellular spaces that are accessible to the cell displaying the class II molecule. HLA class II expressing cells then present peptide antigens derived from the extracellular antigen/bacteria to immune effector cells, including but not limited to, CD4⁺ helper T cells, thereby helping to eliminate such pathogens. The elimination of such pathogens is accomplished by both helping B cells make antibodies against microbes, as well as toxins produced by such microbes, and by activating macrophages to destroy ingested microbes.

HLA class I and class II molecules exhibit extensive polymorphism generated by systematic recombinatorial and point mutation events; as such, hundreds of different HLA types exist throughout the world's population, resulting in substantial immunologic diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, and because of their tremendous diversity, large quantities of individual HLA proteins are required in order to effectively study transplantation, autoimmunity disorders, and for vaccine development.

Antibodies that recognize class I and class II human leukocyte antigens (HLA) currently represent a contraindication at multiple stages of the organ transplant process. Prior to transplantation, patients who have been sensitized to produce HLA-specific antibodies typically wait longer to receive a transplant. Post-transplantation, antibodies that recognize the HLA of the donor organ contribute to hyperacute, acute, and chronic rejection of a transplanted organ. However, it is likely that not all antibodies that recognize HLA promote organ failure. A more thorough understanding of anti-HLA antibodies would therefore indicate those immunoglobulins that are truly a contraindication for transplantation.

It has been difficult to evaluate the phenotypic and functional traits of antibodies to any given HLA molecule because anti-HLA humoral responses tend to be polyclonal and these antibodies cannot be readily isolated for individual characterization. Antibody concentration, isotype, epitope specificity, cross-reactivity, and the ability to fix complement have all been implicated as factors that contribute to the pathogenicity of anti-HLA antibodies (6). More advanced tools such as bead-based semi-quantitative assays have recently provided a more definitive indication for these antibodies' HLA specificity. Nonetheless, the complex nature of human sera and the inability to study antibodies reactive against individual HLA antigens continue to cloud the contribution of antibody isotype, concentration, and specificity to transplant rejection.

The current methods of antibody removal only remove antibodies of broad specificity. The PROSORBA® (Cypress Bioscience, San Diego, Calif.) and follow-on IMMUNOSORBA® (Fresenius Medical Care, Waltham, Mass.) products (and others like them) use Protein A to bind a broad range of antibodies. Plasma is filtered through the IMMUNOSORBA® device to rid the majority of IgG antibodies from the sera. However, IgG3 and IgM and other subtypes are NOT removed. These current devices provide no method of selecting between “wanted” and “not-wanted” antibodies.

Therefore, there exists a need in the art for improved devices that selectively remove anti-MHC/HLA antibodies from a sample, as well as methods of production and use thereof, that overcome the disadvantages and defects of the prior art.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is a schematic representation of a soluble HLA class II trimolecular complex produced in accordance with the presently disclosed and claimed inventive concept(s).

FIG. 2 is a schematic diagram of a method of producing the soluble HLA (sHLA) class II trimolecular complex (of FIG. 1) in accordance with the presently disclosed and claimed inventive concept(s).

FIG. 3 is a schematic diagram of sHLA class II trimolecular complex production in a hollow fiber bioreactor unit.

FIG. 4 graphically depicts the production of sHLA class II DRB1*0103 produced in transfected cells, demonstrating the ability to scale up production from a T175 flask to a hollow fiber bioreactor unit (CELL PHARM®).

FIG. 5 graphically demonstrates the ability of commercially available monoclonal antibodies (mAb) and patient sera to specifically detect the sHLA DRB1*0103 produced in FIG. 4.

FIG. 6 graphically depicts the ability to produce multiple different sHLA class II complexes from transfected cells in accordance with the presently disclosed and claimed inventive methods.

FIG. 7 graphically depicts production in a bioreactor of milligram quantities of sHLA class II over time.

FIG. 8 demonstrates quantification of sHLA class II DRB*0103/DRA*0101 (produced in FIG. 7) using electrospray mass spectroscopy.

FIG. 9 illustrates the molecular weight results and analysis of the proteins from FIG. 8 and using electrospray ionization TOF mass spectrometry.

FIG. 10 graphically depicts coupling of soluble DRB1*1101 ZP HLA Class II molecule to a solid support and use thereof to facilitate removal of HLA Class II specific antibodies in an ELISA format. Panel A: a diagram of the consecutive absorption matrix ELISA performed for specific antibody removal. Panel B: absorbance and retentate values from 3 different HLA Class II specific mAb antibodies: L243, OL (One Lambda), and 2H11 were subjected to the consecutive absorbance matrix.

FIG. 11 graphically depicts that DRB1*1101-specific human sera was recognized by soluble DRB1*1101 in an ELISA format.

FIG. 12 graphically depicts that soluble DRB1*1101 can be coupled to SEPHAROSE® and used to absorb HLA Class II specific antibody, 9.3F10. Panel A: soluble DRB1*1101 was coupled to SEPHAROSE® Fast Flow and packed into a gravity column. mAb 9.3F10, which has DR reactivity, was passed over the column and flow thru was collected as fractions. Then the mAb was eluted using DEA (diethanolamine) buffer, pH 11.3, was added to the column, and fractions were collected. Panel B: two separate ELISAs for total mouse IgG and human HLA were also performed on the Flow Thru and Eluate to detect specific antibodies versus HLA proteins that might have been eluted off the column.

FIG. 13 graphically depicts that antibodies contained in human sera specific for DRB1*1101 can be removed by a DRB1*1101 specific column. Donor #1 sera was passed over the DRB1*1101 SEPHAROSE® column, and two 2 ml fractions of flow thru were collected. To elute, DEA buffer pH 11.3, was added to the column, and two 2 ml fractions were collected. Panel A: a direct DRB1*1101 ELISA was performed to detect the amount of DRB1*1101 specific antibodies that were left in the flow thru and eluate. Panel B: a total human IgG sandwich ELISA was also performed to evaluate passage of total human IgG.

FIG. 14 graphically depicts that soluble DRB1*1101 coupled SEPHAROSE® is specific for DRB1*1101 and not other DR alleles. Donor #2 sera was passed over the same DRB1*1101 column in the same manner as FIG. 13, and two fractions of the flow thru and one fraction of the eluate were evaluated for multi-allele DR reactivity.

FIG. 15 depicts the nucleic acid (SEQ ID NO:1) and amino acid (SEQ ID NO:2) sequences of a DRA*0101 alpha chain-leucine zipper construct. The highlighted sequence encodes a linker that connects DRA1*0101 allele's sequence to the leucine zipper motif's sequence. The underlined sequence encodes the leucine zipper motif.

FIG. 16 depicts the nucleic acid (SEQ ID NO:3) and amino acid (SEQ ID NO:4) sequences of a DRB1*0401 beta chain-leucine zipper construct. The highlighted sequence encodes a linker that connects DRB1*0401 allele's sequence to the leucine zipper motif's sequence. The underlined sequence encodes the leucine zipper motif.

FIG. 17 depicts the nucleic acid (SEQ ID NO:5) and amino acid (SEQ ID NO:6) sequences of a DRB1*0103 beta chain-leucine zipper construct. The highlighted sequence encodes a linker that connects DRB1*0103 allele's sequence to the leucine zipper motif's sequence. The underlined sequence encodes the leucine zipper motif.

FIG. 18 illustrates the construction of sHLA-DR11. A) The transmembrane domains of the alpha (DRA1*01:01) and beta (DRB1*11:01) chains were deleted and replaced by a 7 amino acid linker followed by leucine zipper ACIDp1(LZA) and leucine zipper BASEp1 (LZB), respectively. B) Amino acid sequences for the mature DRA1*01:01 and DRB1*11:01 constructs. Red letters represent the sequence covered from the MS analysis. Underlined letters show the amino acid sequence for the leucine zipper domains.

FIG. 19 illustrates removal and recovery of L243 with a sHLA-DR11 column. A) A280 values for the fractions obtained from the flow through and elution of the sHLA-DR11 column. B) Class II reactivity of the eluted L243 antibody. The raw MFI for each individual HLA complex tested is shown, and the results are grouped together by loci.

FIG. 20 illustrates the specific removal of anti-HLA-DR11 antibodies using the sHLA-DR11 column. A, B) Representative class II HLA reactivities in the starting sera obtained from two sensitized donors, (A:Donor1, B:Donor2). HLA types are color coded by locus (DR11:black, other DR:shades of blue, DQ: shades of red, DP:green). Data are shown as background corrected MFI (BCMFI). C, D) Anti-HLA reactivity of fractions in the column flow-through and eluate from Donor 1 (C) and Donor 2 (D) were analyzed as in A and B. Each trace shows the reactivity profile for a different class II HLA type as shown in the figure legend. HLA types are color coded as in A and B.

FIG. 21 illustrates removal of complement and non-complement fixing antibodies. A) Complement dependant cytolysis of HLA-DR11 positive cells (C433, C418, C428, C423) using anti-HLA-DR11 antibodies. Mean percent cell death is calculated as described in the materials and methods. Starting serum is shown in blue, flow through in red, and eluate in green. Error bars represent the standard deviation from three independent experiments. Significant differences in mean values are shown and were determined by a one way ANOVA (analysis of variance) with a Turkey post-hoc test (p<0.05). B) Representative fluorescent microscope images used for the quantitative analysis in A. Dead cells are red (ethidium bromide) and viable cells are green (acridine orange).

FIG. 22 illustrates isotype profiles of purified anti-HLA-DR11 antibodies. Antibody isotypes in the starting sera, flow through, and eluate were quantified using a LUMINEX®-based ELISA and expressed as a percentage of total antibody.

FIG. 23 illustrates removal of anti-HLA-DR11 antibodies from sensitized sera. The starting sera from two sensitized donors were tested for class II reactivity using a single antigen bead assay. Once the sera were passed over the sHLA-DR11 column, the flow through, and eluate from the column were tested using the same class II single antigen bead assay.

FIG. 24 illustrates the coupling efficiencies of two different SEPHAROSE® matrices with class I soluble HLA. 1 mg of sHLA-B was added to 1 ml of either CNBr-activated or NHS-activated SEPHAROSE® 4 Fast Flow matrix. The coupling was allowed to react for 1 hour and was terminated. Coupling efficiency is calculated using the following equation: (coupling efficiency=mg starting sHLA/mg sHLA in solution after coupling).

FIG. 25 illustrates the binding capacities of two different SEPHAROSE® matrices for class I soluble HLA. Saturating quantities of pan class I HLA monoclonal antibody W6/32 was run over 1 ml of coupled matrix (1 mg @ 1 mg/ml). The matrix was either CNBr-activated or NHS-activated SEPHAROSE® 4 Fast Flow matrix. The sHLA used in this experiment was sHLA-B*07:02. Binding capacity was determined by measuring the quantity of antibody recovered in the elution. To adjust for variations in coupling efficiencies, the data is shown as μg of W6/32 in the elution per mg of sHLA coupled on the matrix.

FIG. 26 illustrates the regeneration capabilities of two different SEPHAROSE® matrices loaded with class I soluble HLA. Saturating quantities of pan class I HLA monoclonal antibody W6/32 was run over 1 ml of coupled matrix (1 mg @ 1 mg/ml). The matrix was either CNBr-activated or NHS-activated SEPHAROSE® 4 Fast Flow matrix. The columns were then serially loaded and eluted 5 times as indicated on the x axis. Percent of the original (cycle 1) antibody binding capacity is shown for each cycle.

FIG. 27 illustrates the coupling efficiencies of two different SEPHAROSE® matrices with class II soluble HLA. 1 mg of sHLA-DR11 was added to 1 ml of either CNBr activated or NHS activated SEPHAROSE® 4 Fast Flow matrix. The coupling was allowed to react for 1 hour and was terminated. Coupling efficiency is calculated using the following equation: (coupling efficiency=mg starting sHLA/mg sHLA in solution after coupling).

FIG. 28 illustrates the binding capacities of two different SEPHAROSE® matrices for class II soluble HLA. Saturating quantities of pan HLA-DR monoclonal antibody L243 was run over 1 ml of coupled matrix (1 mg @ 1 mg/ml). The matrix was either CNBr-activated or NHS-activated SEPHAROSE® 4 Fast Flow matrix. The sHLA used in this experiment was sHLA-DR11. Binding capacity was determined by measuring the quantity of antibody recovered in the elution. To adjust for variations in coupling efficiencies, the data is shown as μg of L243 in the elution per mg of sHLA coupled on the matrix.

FIG. 29 illustrates the regeneration capabilities of two different SEPHAROSE® matrices loaded with class II soluble HLA. Saturating quantities of pan HLA-DR monoclonal antibody L243 was run over 1 ml of coupled matrix (1 mg @ 1 mg/ml). The matrix was either CNBr-activated or NHS-activated SEPHAROSE® 4 Fast Flow matrix. The columns were then serially loaded and eluted 5 times as indicated on the x axis. Percent of the original (cycle 1) antibody binding capacity is shown for each cycle.

FIG. 30 illustrates monoclonal anti-HLA antibody depletion from PBS using a class I HLA SHARC (soluble HLA antibody removal column). Saturating quantities of pan class I HLA monoclonal antibody W6/32 was run over 65 ml of coupled matrix (24.4 mg at 97 μg/ml). The column was then washed with PBS pH 7.4 and eluted with 0.1 M Glycine pH 11. During the load and wash phase, 11.7 mg passed through the column. During the elution phase, 8 mg of antibody was recovered.

FIG. 31 illustrates polyclonal anti-HLA-A2 antibody depletion from patient plasma with class I HLA-A2 SHARC. 2.5 L of Patient plasma containing anti-HLA antibodies was run over the 65 ml sHLA-A2 SHARC. Plasma pre- and post-SHARC were analyzed using a multiplexed, LUMINEX®-based detection method as described by the manufacturer (LABScreen® Single Antigen, OneLambda, Inc., Canoga Park, Calif.). This individual had multiple HLA specificities, as indicated in the legend. As shown in the figure, anti-HLA-A2 antibodies, as well as serologically related antibodies (B57, B58), were reduced from the starting plasma. Serologically unrelated anti-HLA antibodies (B61, B81, B18, B60) were unchanged from the pre-SHARC plasma as they passed through the SHARC. This demonstrates the specificity of the HLA-A2 SHARC.

FIG. 32 illustrates polyclonal anti-HLA-A2 antibody depletion from patient plasma with HLA-A2 SHARC. 2.5 L of Patient plasma containing anti-HLA antibodies was run over the 65 ml sHLA-A2 SHARC. Fractions were collected as the plasma was passed over the SHARC. The resulting fractions were analyzed using a multiplexed, LUMINEX®-based detection method as described by the manufacturer. Data is represented by percent reduction in BCMFI (% Reduction in BCMFI=1−(BCMFI of the fraction/BCMFI starting plasma).

FIG. 33 illustrates monoclonal anti-HLA antibody depletion from PBS using a class II HLA SHARC (soluble HLA antibody removal column). Saturating quantities of pan HLA-DR monoclonal antibody L243 was ran over 65 ml of coupled matrix (30.0 mg @ 120 μg/ml). The column was then washed with PBS pH 7.4 and eluted with 0.1 M Glycine pH 11. During the load and wash phase, 2 mg passed through the column. During the elution phase, 23.1 mg of antibody was recovered.

FIG. 34 illustrates polyclonal anti-HLA-DR11 antibody depletion from patient plasma with HLA-DR11 SHARC. 2.5 L of Patient plasma containing anti-HLA antibodies was run over the 65 ml sHLA-DR11 SHARC. Plasma pre- and post-SHARC were analyzed using a multiplexed, LUMINEX®-based detection method as described by the manufacturer (LABScreen® Single Antigen, OneLambda, Inc., Canoga Park, Calif.). This individual had multiple HLA specificities as indicated in the legend. As shown in the figure, anti-HLA-DR11 antibodies as well as serologically related antibodies (DR13, DR4, DR17) were reduced from the starting plasma. Serologically unrelated anti-HLA antibodies (DQ7, DQ8, DQ9) were unchanged from the pre-SHARC plasma as they passed through the SHARC. This demonstrates the specificity of the HLA-DR11 SHARC.

FIG. 35 illustrates polyclonal anti-HLA-DR11 antibody depletion from patient plasma with HLA-DR11 SHARC. 2.5 L of Patient plasma containing anti-HLA antibodies was run over the 65 ml sHLA-DR11 SHARC. Fractions were collected as the plasma was passed over the SHARC. The resulting fractions were analyzed using a multiplexed, LUMINEX®-based detection method as described by the manufacturer. Data is represented by percent reduction in BCMFI (% Reduction in BCMFI=1−(BCMFI of the fraction/BCMFI starting plasma).

FIG. 36 illustrates the coupling efficiency of soluble class I HLA A*0201 to an NHS-activated SEPHAROSE® Fast Flow Matrix column.

FIG. 37 illustrates a repeatability study evaluating the column profile of FIG. 36 based on absorption units (mAU) to detect proteinaceous material.

FIG. 38 illustrates a repeatability study evaluating the column profile of FIG. 36 based on pH.

FIG. 39 illustrates a repeatability study evaluating the column profile of FIG. 36 based on conductivity to detect changes in buffer phases.

FIGS. 40-42 illustrate a stability evaluation of the column of FIG. 36, wherein the column was exposed to multiple rounds of load-elute-equilibrate cycles with W6/32.

FIG. 43 illustrates a capacity evaluation of the column of FIG. 36, utilizing varying amounts of W6/32.

FIG. 44 illustrates a capacity evaluation of the column of FIG. 36, utilizing varying amounts of Anti-β2m.

FIG. 45 illustrates a capacity evaluation of the column of FIG. 36, utilizing varying amounts of Ant-VLDL (an antibody against an artificial tail introduced into the A*0201 molecule).

FIG. 46 illustrates a binding efficiency evaluation of the column of FIG. 36, using W6/32.

FIG. 47 illustrates a binding efficiency evaluation of the column of FIG. 36, using Anti-β2m.

FIG. 48 illustrates a binding efficiency evaluation of the column of FIG. 36, using Anti-VLDL.

FIG. 49 illustrates a proposed application scenario in accordance with one embodiment of the presently disclosed and claimed inventive concept(s).

FIG. 50 illustrates the specific depletion and recovery of DR11 alloantibodies from sensitized sera. Data in A, B, C, is shown as both a histogram as well a heatmap. 1 ml of DR11 sensitized sera was passed over a 1 ml sHLA-DR11 column, washed, and eluted off the column. MFI values of the sera before (A) and after (B) passage over the DR11 column. C) MFI values of the neutralized column eluate. D) MFI values of approximately 160 μl (3 drop) fractions of the flow-through (1 ml of sera followed by 1 ml of PBS). E) MFI values of approximately 160 μl (3 drop) fractions of the elution. In D and E each line represents MFI values from the indicated allomorph.

FIG. 51 illustrates the purification of DR11 specific alloantibodies from multiple patient sera. Heatmap indicating MFI values for each allomorph on the panel for the sera before the column (PRE), after the column (POST), or in the elution (ELUTION) in patients G-12. Scale of the heatmap is shown on the bottom panel. For clarity, values below the threshold are blacked out. HLA-DR11 MFI values are outlined in blue. Threshold values for PRE and POST were determined by taking the average bead MFI of negative sera plus 5 standard deviations. Threshold values for ELUTION were determined by taking the average DQ bead MFI plus 5 standard deviations.

FIG. 52 illustrates CDC activity of purified DR11 specific alloantibodies. A) Sera from patient ‘G’ was passed over the column and the eluted antibodies were collected. These three samples (PRE, POST, ELUTION) were then added to 4 different HLA-DR11 positive B-cells (C433 blue, C418 red, C428 green, C432 purple) in the presence of complement. Cell death was measured according to the materials and methods and is shown in the histogram. A representative image of the assay (cell line C428) is shown below the histogram. Class II haplotype of each cell line is shown in the table inlay. B) Complement dependant cell death of the eluted antibodies from patients G-12.

FIG. 53 illustrates the isotype profiles of the purified DR11 specific alloantibodies. A) Isotype profiles of the purified antibodies from all of the patients in the study. Seven different isotypes were analyzed: IgG1 (blue), IgG2 (red), IgG3 (green), IgG4 (purple), IgM (teal), IgA (orange), and IgE (light blue). B) Proportion of indicated isotype in purified HLA-DR11 antibodies compared to bulk serum antibodies. Line represents the median value and bars show the interquartile range. P values are shown as the result of a Mann-Whitney t-test.

FIG. 54 illustrates the correlation of DR11 alloantibody concentration and MFI values. MFI values of 13 different patients plotted against the total Ig (A) or IgG1-4 (B, C). Data was fit to linear model and shown with the 95% confidence bands. R² values for each line are shown.

FIG. 55 illustrates SEC-HPLC of purified DR11 alloantibodies. Approximately 10 μg of purified human IgM (bule), IgA (red), and IgG (black) were either left neat (A) or reduced with 100 mM DTT (E) and run individually over a size-exclusion column. B-D) 10 μg of neat DR11 alloantibodies from patients 13 (B), 14 (C), 15 (D), was run over a size-exclusion column. The collected Ig monomeric fraction is shaded in grey. F-H) 10 μg of DTT reduced DR11 alloantibodies from patients 13 (B), 14 (C), 15 (D), was run over a size-exclusion column. The collected Ig monomeric fraction is shaded in grey.

FIG. 56 illustrates the cross-reactive pattern of multimeric and monomeric alloantibodies. HLA-DR MFI values of the different antibody preparations. For patients 13, 14, and 15 MIF values were determined for native antibodies (All), monomeric antibodies (Mono), or DTT treated monomeric antibodies (DTT) at a saturating concentration of 200 μg/ml. MFI vales are shown as a heat map and scale is shown to the right.

FIG. 57 illustrates isotype specificity of the One Lambda human IgG secondary antibody. Single isotype antibodies were biotynlated and coupled to LUMINEX® beads coated in streptavidin (Lumavidin Microspheres) where a single isotype was on a single distinct bead number. Single isotype beads were then mixed and stained with the anti-human IgG PE secondary antibody (diluted ten-fold) supplied by One Lambda. Raw MFI values are shown. IgG is a mixture of all four IgG subclasses at their naturally occurring ratios.

FIG. 58 illustrates the proportion of isotypes in SEC-HPLC fractions. For patients 13, 14, and 15, the SEC-HPLC fraction containing monomeric Ig, ‘Monomeric Ig’ (FIG. 6B-D grey shaded area), and the antibodies before fractionation, ‘All Ig’, were assessed for their isotype profile according to the materials and methods. Percent Ig is shown either by IgG1-4 (blue) or IgM, A, E (red).

FIG. 59 illustrates soluble phase inhibition. HLA antibody inhibition dependant on the HLA protein used: HLA A2, B7, B13 proteins (0.05 μg/μl) were added to patient serum and the extent of HLA antibody inhibition was determined. The experiment was performed at 22° C. for 30 minutes. HLA B7 and B13 share the 163E-166E epitope and give effective inhibition, illustrating shared epitope reactivity. In contrast, HLA A2 which lacks the epitope causes no inhibition.

FIG. 60 illustrates soluble phase inhibition. A) HLA-A2: specific reactions are shaded together with HLA A69 which share 107W and marked with *. Epitope specific removal>75% is observed. B) HLA-A24: specific reactions (shaded bars) depleted all reactions against HLA-Bw4 included HLA-A specificities carrying the epitope (marked *). C) HLA-B57: removes the same Bw4 associated specificities as HLA-A24 but removal efficacy increased (typically>50%). D) HLA-Cw2: confirms the expected removal of all HLA-C locus specificities carrying the 77N+80K epitope. E) Combination of all four proteins: In soluble phase provides a very effective overall reduction in HLA reactive repertoire, with median antibody reduction of 72.3%.

FIG. 61 illustrates HLA protein bound to sepharose (solid phase). A) HLA-A2: epitope specific reduction in the region of 60-70%. B) HLA-A24: specific reduction of all Bw4 associated specificities. C) HLA-B57: epitope specific reduction of all Bw4 associated specificities with increased efficacy compared to HLA-A24 (50-60% vs 30-40%). D) HLA-Cw2: Specific reduction, approximately 50%, of all Cw specificities carrying 77N+80K epitope. E) Combination of all four proteins: Consistent epitope specific removal of all HLA reactive specificities with median antibody reduction of 73.6%.

### DETAILED DESCRIPTION OF THE INVENTIVE CONCEPT(S)

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first”, “second”, “third”, “fourth”, etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein, “substantially pure” means an object species is the predominant species present (i.e., on a molar basis it is more abundant than any other individual species in the composition). Generally, a substantially pure composition will comprise more than about 50% percent of all macromolecular species present in the composition, such as more than about 55%, 60%, 65%, 70%, 75%, 80%, 85%, 90%, 95%, and 99%. In one embodiment, the object species is purified to essential homogeneity (contaminant species cannot be detected in the composition by conventional detection methods) wherein the composition consists essentially of a single macromolecular species.

The terms “isolated polynucleotide” and “isolated nucleic acid segment” as used herein shall mean a polynucleotide of genomic, cDNA, or synthetic origin or some combination thereof, which by virtue of its origin the “isolated polynucleotide” or “isolated nucleic acid segment” (1) is not associated with all or a portion of a polynucleotide in which the “isolated polynucleotide” or “isolated nucleic acid segment” is found in nature, (2) is operably linked to a polynucleotide which it is not linked to in nature, or (3) does not occur in nature as part of a larger sequence.

The term “isolated protein” referred to herein means a protein of genomic, cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or, (4) does not occur in nature.

The term “polypeptide” as used herein is a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “antibody” is used in the broadest sense, and specifically covers monoclonal antibodies (including full length monoclonal antibodies), polyclonal antibodies, multispecific antibodies (e.g., bispecific antibodies), and antibody fragments (e.g., Fab, F(ab′)2 and Fv) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “MHC” as used herein will be understood to refer to the Major Histocompability Complex, which is defined as a set of gene loci specifying major histocompatibility antigens. The term “HLA” as used herein will be understood to refer to Human Leukocyte Antigens, which is defined as the major histocompatibility antigens found in humans. As used herein, “HLA” is the human form of “MHC”.

The terms “MHC class I light chain” and “MHC class I heavy chain” as used herein will be understood to refer to portions of the MHC class I molecule. Structurally, class I molecules are heterodimers comprised of two noncovalently bound polypeptide chains, a larger “heavy” chain (α) and a smaller “light” chain (β-2-microglobulin or β2m). The polymorphic, polygenic heavy chain (45 kDa), encoded within the MHC on chromosome six, is subdivided into three extracellular domains (designated 1, 2, and 3), one intracellular domain, and one transmembrane domain. The two outermost extracellular domains, 1 and 2, together form the groove that binds antigenic peptide. Thus, interaction with the TCR occurs at this region of the protein. The 3^{rd} extracellular domain of the molecule contains the recognition site for the CD8 protein on the CTL; this interaction serves to stabilize the contact between the T cell and the APC. The invariant light chain (12 kDa), encoded outside the MHC on chromosome 15, includes a single, extracellular polypeptide. The terms “MHC class I light chain”, “β-2-microglobulin”, and “β2m” may be used interchangeably herein. Association of the class I heavy and light chains is required for expression of class I molecules on cell membranes.

Like MHC class I molecules, class II molecules are also heterodimers, but in this case consist of two nearly homologous α and β chains, both of which are encoded in the MHC. The class II MHC molecules are membrane-bound glycoproteins, and both the α and β chains contain external domains, a transmembrane anchor segment, and a cytoplasmic segment. Each chain in a class II molecule contains two external domains: the 33-kDa a chain contains α₁ and α₂ external domains, while the 28-kDa β chain contains β₁ and β₂ external domains. The membrane-proximal α₂ and β₂ domains, like the membrane-proximal 3^{rd} extracellular domain of class I heavy chain molecules, bear sequence homology to the immunoglobulin-fold domain structure. The membrane-distal domain of a class II molecule is composed of the α₁ and β₁ domains, which form an antigen-binding cleft for processed peptide antigen. The peptides presented by class II molecules are derived from extracellular proteins (not cytosolic intracellular peptide antigens as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane. Because the peptide-binding groove of MHC class II molecules is open at both ends while the corresponding groove on class I molecules is closed at each end, the peptides presented by MHC class II molecules are longer, generally between 13 and 24 amino acid residues long. Like class I HLA, the peptides that bind to class II molecules often have internal conserved “motifs”, but unlike class I-binding peptides, they lack conserved motifs at the carboxyl-terminal end, since the open ended binding cleft allows a bound peptide to extend from both ends.

The term “trimolecular complex” as used herein will be understood to refer to the MHC heterodimer associated with a peptide. An “MHC class I trimolecular complex” or “HLA class I trimolecular complex” will be understood to include the class I heavy and light chains associated together and having a peptide displayed in an antigen binding groove thereof. The terms “MHC class II trimolecular complex” and “HLA class II trimolecular complex” will be understood to include the class II alpha and beta chains associated together and having a peptide displayed in an antigen binding groove thereof.

The term “MHC moiety” as used herein will be understood to include MHC class I trimolecular complexes, MHC class II trimolecular complexes, and any portion or subunit of MHC class I/class II molecules.

The term “biological sample” as used herein will be understood to include, but not be limited to, serum, tissue, blood, plasma, cerebrospinal fluid, tears, saliva, lymph, dialysis fluid, organ or tissue culture derived fluids, and fluids extracted from physiological tissues. The term “biological sample” as used herein will also be understood to include derivatives and fractions of such fluids, as well as combinations thereof. For example, the term “biological sample” will also be understood to include complex mixtures.

The term “HLA protein” as used herein will be understood to refer to any HLA molecule, complex thereof or fragment thereof that is capable of being expressed on a surface of a non-human cell. Examples of HLA proteins that may be utilized in accordance with the presently disclosed and claimed inventive concept(s) include, but are not limited to, an HLA class I trimolecular complex, an HLA class II trimolecular complex, an HLA class II α chain and an HLA class II β chain. Specific examples of HLA class II α and/or β proteins that may be utilized in accordance with the presently disclosed and claimed inventive concept(s) include, but are not limited to, those encoded at the following gene loci: HLA-DRA; HLA-DRB1; HLA-DRB3,4,5; HLA-DQA; HLA-DQB; HLA-DPA; and HLA-DPB.

The term “mammalian cell” as used herein will be understood to refer to any cell capable of expressing a recombinant HLA protein (as defined herein above). Therefore, any “mammalian cell” utilized in accordance with the presently disclosed and claimed inventive concept(s) must contain the necessary machinery and transport proteins required for expression of MHC/HLA proteins and/or MHC/HLA trimolecular complexes on a surface of such cell. “Mammalian cells” utilized in accordance with the presently disclosed and claimed inventive concept(s) must have (A) machinery for chaperoning and loading MHC/HLA proteins, such as class I and class II proteins; and (B) such machinery must be able to interact and work with human HLA proteins, such as class I and class II proteins. Not all cells express class II MHC protein; only professional immune cells such as but not limited to dendritic cells (DC), macrophages, B cells, and the like express class II proteins. Therefore, when it is desired to express HLA class II protein in a mammalian, non-human cell, such non-human cell must express class II MHC for that species and contain the appropriate machinery for interacting and working with both that species' class II MHC as well as human HLA class II. However, the presently disclosed and claimed inventive concept(s) also includes the use of cells of other lineages that have been induced to express class II MHC, such as but not limited to, cytokines, cells that have been subjected to mutagenesis, and the like.

The term “mammalian cell” as used herein refers to immortalized mammalian cell lines and does not include animals or primary cells. Examples of “mammalian cells” that may be utilized in accordance with the presently disclosed and claimed inventive concept(s) include, but are not limited to, human and mouse DC lines, macrophage lines, and B cell lines.

MHC (major histocompatibility complex) or HLA (Human leukocyte antigen) Class II molecules are found only on a few specialized cell types, including macrophages, dendritic cells and B cells, all of which are professional antigen-presenting cells (APCs). The peptides presented by class II molecules are derived from extracellular proteins (not cytosolic as in class I); hence, the MHC class II-dependent pathway of antigen presentation is called the endocytic or exogenous pathway. Loading of class II molecules must still occur inside the cell; extracellular proteins are endocytosed, digested in lysosomes, and bound by the class II MHC molecule prior to the molecule's migration to the plasma membrane.

Like MHC class I molecules, class II molecules are also heterodimers, but in this case consist of two homologous peptides, an α and β chain, both of which are encoded in the MHC. Class II molecules are composed of two polypeptide chains, both encoded by the D region. These polypeptides (alpha and beta) are about 230 and 240 amino acids long, respectively, and are glycosylated, giving molecular weights of about 33 kDa and 28 kDa. These polypeptides fold into two separate domains; alpha-1 and alpha-2 for the alpha polypeptide, and beta-1 and beta-2 for the beta polypeptide. Between the alpha-1 and beta-1 domains lies a region very similar to that seen on the class I molecule. This region, bounded by a beta-pleated sheet on the bottom and two alpha helices on the sides, is capable of binding (via non-covalent interactions) a small peptide. Because the antigen-binding groove of MHC class II molecules is open at both ends while the corresponding groove on class I molecules is closed at each end, the antigens presented by MHC class II molecules are longer, generally between 15 and 24 amino acid residues long. This small peptide is “presented” to a T-cell and defines the antigen “epitope” that the T-cell recognizes.

Turning now to the presently disclosed and claimed inventive concept(s), anti-MHC antibody removal devices, as well as kits containing same, and methods of production and use thereof, are disclosed and claimed herein. The devices/kits described herein may be utilized for various clinical, diagnostic and therapeutic methods, as described in more detail herein below. The anti-MHC antibody removal device includes a soluble MHC moiety covalently coupled to a solid support. The soluble MHC moiety attached to the solid support is serologically active such that the soluble MHC moiety maintains the physical, functional and antigenic integrity of a native MHC trimolecular complex. When a biological sample is brought into contact with the anti-MHC antibody removal device, anti-MHC antibodies specific for the MHC moiety attach to the soluble MHC moiety and are detected and/or removed from the biological sample.

The soluble MHC moiety may be a class I or class II soluble MHC moiety produced by any methods known in the art or otherwise contemplated herein. In certain embodiments, the soluble MHC moiety is a class I or class II soluble HLA moiety. Non-limiting examples of class I soluble HLA moieties that may be utilized in accordance with the presently disclosed and claimed inventive concept(s) (as well as methods of production and purification thereof) are disclosed in U.S. Ser. No. 09/465,321, filed Dec. 17, 1999; U.S. Ser. No. 10/022,066, filed Dec. 18, 2011 (US Publication No. 2003/0166057, published Sep. 4, 2003); and U.S. Ser. No. 10/337,161, filed Jan. 2, 2011 (US Publication No. 2003/0191286, published Oct. 9, 2033). The entire contents of the above-referenced patent applications are hereby expressly incorporated herein by reference. Non-limiting examples of class II soluble HLA moieties that may be utilized in accordance with the presently disclosed and claimed inventive concept(s) (as well as methods of production and purification thereof) are disclosed in parent application U.S. Ser. No. 12/859,002, filed Aug. 18, 2010, and are disclosed in further detail herein below.

In certain embodiments, the MHC/HLA is purified substantially away from other proteins such that the individual MHC/HLA trimolecular complex maintains the physical, functional and antigenic integrity of a native MHC/HLA trimolecular complex. The functionally active, individual MHC/HLA trimolecular complex may be purified as described herein or by any other method known in the art. Upon attachment to the solid support, the conformation of the functionally active, individual MHC/HLA trimolecular complex is maintained.

Any solid support capable of covalent attachment to the MHC/HLA moiety and capable of otherwise functioning in accordance with the presently disclosed and claimed inventive concept(s) may be utilized. In certain embodiments, the solid support may be selected from the group consisting of a well, a bead (such as but not limited to, flow cytometry bead and/or a magnetic bead), a membrane (such as but not limited to, a nitrocellulose membrane, a PVDF membrane, a nylon membrane, and acetate derivative), a microtiter plate, a matrix (such as a SEPHAROSE® matrix), a pore, plastic, glass, a polymer, a polysaccharide, nylon, nitrocellulose, a paramagnetic compound, and combinations thereof. A non-limiting example of a solid support capable of functioning in accordance with the presently disclosed and claimed inventive concept(s) includes a device (such as a column) that possesses an inlet, an outlet, and a chamber disposed therebetween. The chamber contains an inner surface on which the serologically active soluble MHC moiety is disposed, whereby the inlet is disposed to introduce the biological sample into the chamber. As the biological sample flows through the device, anti-MHC antibodies specific for the serologically active MHC moiety attach thereto and are removed from the biological sample. The flow through collected from the outlet is substantially free of anti-MHC antibodies specific for the serologically active MHC moiety. Particular non-limiting examples of devices of this type include human use devices (HUDs), such as an extracorporeal plasmapheresis HUD.

In certain embodiments, NHS-activated SEPHAROSE® matrix is utilized as the solid support. This matrix immobilizes proteins by covalent attachment of their primary amino groups to the NHS (N-hydroxysuccinimide) activated group to form a very stable amide linkage. This is an important feature for therapeutic uses for the devices and methods described herein, as it prevents leaching of the immobilized MHC/HLA complexes from the substrate/solid support during a therapy (such as but not limited to, the use of the device as an extracorporeal device); leaching of these molecules (as well as fragments and/or subunits thereof) could cause deleterious effects to a patient. In addition to increased stability, the NHS-activated SEPHAROSE® matrix also exhibits increased binding capacity resulting from a 14 atom spacer arm present therein; the spacer arm allows the MHC/HLA to reposition as necessary and thus provide better contact with antibodies.

In certain other embodiments, alternative coupling linkages are utilized. Non-limiting examples of other types of linkages include sugar chemistry, carboxy linkage, sulfur linkage, or any other type of linkage chemistry known in the art or otherwise available to a person having ordinary skill in the art that would allow the coupling of an MHC moiety to a solid support.

In certain embodiments, the presently disclosed and claimed inventive concept(s) uses soluble HLA class I trimolecular complexes produced by the methods described in the US patents/patent applications cited herein above. In a non-limiting example, soluble HLA class I trimolecular complexes that are purified substantially away from other proteins such that the individual soluble class I MHC trimolecular complexes maintain the physical, functional and antigenic integrity of the native class I MHC trimolecular complex are provided. The trimolecular complex comprises a recombinant, individual soluble class I MHC heavy chain molecule, beta-2-microglobulin non-covalently associated with the individual soluble class I MHC heavy chain molecule, and a peptide endogenously loaded in an antigen binding groove of the individual soluble class I MHC heavy chain molecule. These molecules are produced by providing a nucleotide segment encoding a desired individual class I MHC heavy chain that has the coding regions encoding the cytoplasmic and transmembrane domains of the desired individual class I MHC heavy chain allele removed such that the nucleotide segment encodes a truncated, soluble form of the desired individual class I MHC heavy chain molecule. This nucleotide segment may be synthetically produced, or it may be produced by locus-specific PCR amplification of the truncated allele (either from cDNA that has been reverse transcribed from mRNA isolated from a source, or directly from gDNA). The nucleotide segment is then cloned into a mammalian expression vector, thereby forming a construct that encodes the desired individual soluble class I MHC heavy chain molecule. A mammalian cell line is then transfected with the construct to provide a mammalian cell line expressing a construct that encodes a recombinant, individual soluble class I MHC heavy chain molecule, wherein the mammalian cell line is able to naturally process proteins into peptide ligands for loading into antigen binding grooves of MHC molecules, and wherein the mammalian cell line expresses beta-2-microglobulin. The mammalian cell line is then cultured under conditions which allow for expression of the recombinant individual soluble class I MHC heavy chain molecule from the construct, such conditions also allowing for endogenous loading of a peptide ligand into the antigen binding groove of each recombinant, individual soluble class I MHC heavy chain molecule and non-covalent association of native, endogenously produced beta-2-microglobulin to form the individual soluble class I MHC trimolecular complexes prior to secretion of the individual soluble class I MHC trimolecular complexes from the cell. The soluble class I MHC trimolecular complexes are then harvested from the culture while retaining the mammalian cell line in culture for production of additional soluble class I MHC trimolecular complexes, and the individual, soluble class I MHC trimolecular complexes are purified substantially away from other proteins, wherein the individual soluble class I MHC trimolecular complexes maintain the physical, functional and antigenic integrity of the native class I MHC trimolecular complex, and wherein each trimolecular complex so purified comprises identical recombinant, individual soluble class I MHC heavy chain molecules.

In other embodiments, the presently disclosed and claimed inventive concept(s) uses soluble HLA class II trimolecular complexes produced by the methods described herein that provide advancements in the areas of purity, quantity, and applications over existing methods; these methods use recombinant DNA methods to alter the protein in a manner that allows mammalian host cells to secrete the protein. HLA class II is naturally produced as a trimolecular complex that is endogenously loaded with peptide ligands and is bound to the membrane. Obtaining such naturally processed and loaded class II presently primarily proceeds by gathering membrane bound forms. Production of membrane bound class II requires cell populations to be lysed for capture of the complex. This method is known as cell lysate and represents state-of-the-art for natural mammalian HLA production for anti-HLA antibody detection assays. Cell lysate class II products are a mixture of numerous cell surface components, including the membrane anchored HLA class II trimolecular complex and other non-HLA proteins that decorate the cell membrane and that co-purify with HLA. Isolation of the HLA from other cell debris and membrane proteins reduces the yield of HLA class II. When producing HLA class II from detergent lysates, one is faced with either contaminating cell surface proteins and/or low class II protein yield. As an alternative, HLA class II can be obtained from Drosophila Schneider S-2 (insect) cell lines (Novak et al., 1999; and U.S. Pat. No. 7,094,555 issued to Kwok et al. on Aug. 22, 2006) and P. pastoris (yeast) (Kalandadze et al. 1996), whereby soluble forms of the HLA class II molecule have been produced. However, class II produced in insect cells lack the endogenously loaded peptides that are an integral component of the HLA class II native trimolecular complex. The HLA molecules produced in insect cells also lack the native glycosylation of mammalian cells. As insect cells lack mammalian protein glycosylation mechanisms and lack the chaperone complexes needed for natural peptide ligand loading, there is a reluctance to utilize class II proteins from insects for clinical applications.

Thus, certain embodiments of the presently disclosed and claimed inventive concept(s) use HLA class II produced by secretion from mammalian cells as a means to produce a native trimolecular complex free of contaminating membrane proteins. Through HLA class II secretion from mammalian cells, a pure product in which the predominant species is the desired HLA class II trimolecular complex is produced. A pure, secreted molecule simplifies and enables downstream purification. Soluble HLA complexes are conducive to hollow fiber bioreactor production systems, such as but not limited to, the CELL PHARM® system (McMurtrey et al. 2008; Hickman et al., 2003; and Prilliman et al., 1999), as well as other systems designed for recombinant native protein secretion from mammalian cells. Highly concentrated harvests are much “cleaner” than cell lysates, thus allowing for minimal product loss because purification is simplified.

Other embodiments of the presently disclosed and claimed inventive concept(s) may utilize HLA class II trimolecular complexes in native form that have been produced and purified via cell lysate methods; however, the complexes produced by these prior art methods have varying amounts of cell membrane secured to the purified HLA product, thereby creating several challenges for the yield of a homogeneous HLA product as well as problems associated with the use thereof.

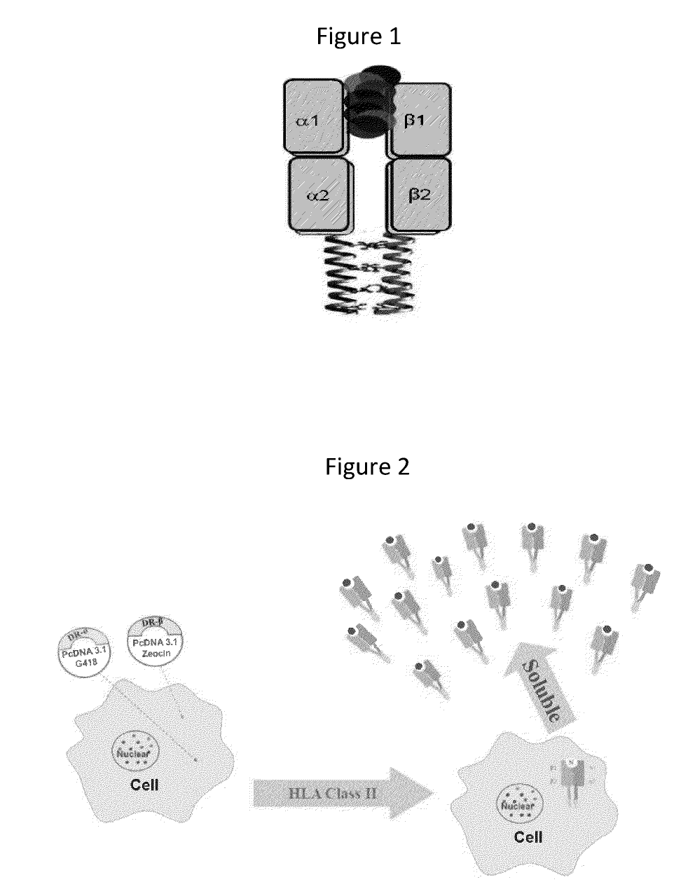

The presently disclosed and claimed inventive concept(s) includes the use of soluble HLA class II trimolecular complexes produced in mammalian cells by a method that solves, in a unique and novel manner, the limitations seen when using cell lysate and insect cell techniques (FIG. 2 illustrates the method of production, while FIG. 1 represents the sHLA trimolecular complexes produced by said method). This production method overcomes the disadvantages and defects of the prior art through the use of a combination of elements; first, each of the α and β chains of the HLA class II complex is truncated such that the domain normally anchoring the complex to the cell surface is removed by recombinant DNA techniques. In native form, the alpha and beta chains of the HLA class II trimolecular complexes rely on the transmembrane domain to maintain a native conformation. While removal of this transmembrane domain facilitates secretion, this removal prevents formation of a trimolecular complex. The sHLA production method removes the transmembrane domain and replaces it with a super secondary structural motif, such as but not limited to, a leucine zipper protein sequence, which serves as a tethering moiety for the class II alpha and beta chains. The super secondary structural motif (such as but not limited to, a leucine zipper) thereby creates adhesion or fusion forces between proteins.

The sHLA production method may further include the recombinant production of the soluble alpha and beta chains of the desired HLA class II in a mammalian cell line. The use of a recombinant mammalian cell line provides two distinct advantages over the prior art: first, production in a mammalian cell line allows the alpha and beta chains of the HLA class II molecule to be glycosylated in the same manner as seen for native HLA class II alpha and beta chains. Second, the mammalian cell line contains the appropriate machinery for natural endocytosis and lysosomal digestion to produce the same peptide ligands as would be produced by a native cell (referred to herein as an “endogenously produced peptide ligand”), as well as the appropriate chaperone machinery for trafficking and loading of the endogenously produced peptide ligands into an antigen binding groove formed between the alpha and beta chains of the HLA class II molecule.

Therefore, the features of (a) glycosylated, soluble HLA class II α and β chains; (b) production in a non-human mammalian cell line (or a human cell line that does not express endogenous class II molecules); and (c) a non-covalently attached, endogenously produced peptide ligand, provide distinct advantages that overcome the disadvantages and defects of the prior art cell lysate and non-mammalian cell production methods.

Endogenously loaded class II is a key element that distinguishes from the prior art. The endogenous peptide allows the class II trimolecular complex to be used in multiple applications not previously possible in soluble forms of the prior art (U.S. Pat. No. 7,094,555, previously incorporated herein by reference; Novak et al., 1999; and Kalandadze et al., 1996). Regarding the currently claimed application method, only a HLA class II in its native trimolecular complex form can properly bind HLA class II specific antibodies. Similarly, the effects of a non-glycosylated HLA molecule on the conformation of class II antibody epitopes when used for HLA specific antibody detection or T-cell solicitation are unknown, but there is some evidence that improper glycosylation disrupts antigen presentation (Guerra et al., 1998). Therefore, the most advantageous format for HLA class II production is to maintain all components in a native form. It has been shown that HLA specific antibody recognition is impacted indirectly by the peptides that are part of the class I complexes (Wilson, 1981). The native binding of HLA specific antibodies is a key element of the presently disclosed and claimed inventive concept(s) when the sHLA described and claimed herein is used as the antigen in an HLA antibody sera screening/removal assay.

In certain embodiments, the presently disclosed and claimed inventive concept(s) utilizes sMHC/sHLA produced by the method described herein below. In the method, a first isolated nucleic acid segment is provided, wherein the first isolated nucleic acid segment encodes a soluble form of an alpha chain of at least one HLA class II molecule, and a second isolated nucleic acid segment is provided, wherein the second isolated nucleic acid segment encodes a soluble form of a beta chain of the at least one HLA class II molecule. The isolated nucleic acid segments may be present in a single recombinant vector, or the isolated nucleic acid segments may be present on two separate recombinant vectors. The coding regions encoding the transmembrane domains of the alpha and beta chains have been removed and replaced with a super secondary structural motif that enables the alpha and beta chains (which previously interacted through their transmembrane domains) to interact. In one embodiment, the super secondary structural motif is a leucine zipper protein sequence that acts as a tethering moiety for the alpha and beta chains.

The isolated nucleic acid segments may be provided by any methods known in the art, including commercial production of synthetic segments. In one embodiment, the nucleic acid segments may be provided by a method that includes the steps of PCR amplification of the alpha and beta alleles from genomic DNA or cDNA. Methods of obtaining gDNA or cDNA for PCR amplification of MHC are described in detail in the inventor's earlier applications U.S. Ser. No. 10/022,066, filed Dec. 18, 2001 and published as US 2003/0166057 A1 on Sep. 4, 2003; and U.S. Pat. No. 7,521,202, issued Apr. 21, 2009; the entire contents of which are hereby expressly incorporated herein by reference. Therefore, while the following non-limiting example begins with gDNA and utilizes PCR amplification, it is to be understood that the scope of the presently disclosed and claimed inventive concept(s) is not to be construed as limited to any particular starting material or method of production, but rather includes any method of providing an isolated nucleic acid segment known in the art.

In one particular embodiment, gDNA is obtained from a sample, wherein portions of the gDNA encode a desired individual HLA class II molecule's alpha chain and beta chain. Two PCR products are then produced: a first PCR product encoding a soluble form of the desired HLA class II alpha chain, and a second PCR product encoding a soluble form of the desired HLA class II beta chain. Each of the PCR products is produced by PCR amplification of the gDNA, wherein the amplifications utilize at least one locus-specific primer having a leucine sequence incorporated into a 3′ primer, thereby resulting in PCR products that do not encode the cytoplasmic and transmembrane domains of the desired HLA class II alpha or beta chains and thus produce PCR products that encode soluble HLA class II alpha or beta chains. The 3′ primer utilized for PCR amplification of the HLA class II alpha chain may incorporate the leucine sequence consistent with the acid sequence of the leucine zipper dimer, while the 3′ primer utilized for PCR amplification of the HLA class II beta chain may incorporate the leucine sequence consistent with the basic sequence of the leucine zipper dimer. However, it is to be understood that the description of the leucine zipper moiety is for purposes of example only, and that the presently disclosed and claimed inventive concept(s) encompasses the use of any super secondary structural motif that enables the alpha and beta chains (which previously interacted through their transmembrane domains) to interact.

Once the isolated nucleic acid segments are provided, they are then inserted into at least one mammalian expression vector to form at least one plasmid containing the PCR products encoding the soluble HLA class II alpha chain and the soluble HLA class II beta chain. It is to be understood that the two nucleic acid segments may be inserted into the same vector or separate vectors.

The plasmid(s) containing the two PCR products are then inserted into at least one suitable immortalized, mammalian host cell line, wherein the cell line contains the necessary machinery and transport proteins required for expression of HLA proteins and/or are able to naturally process proteins into peptide ligands capable of being loaded into antigen binding grooves of HLA class II molecules.

The cell line is then cultured under conditions which allow for expression of the individual soluble HLA class II alpha and beta chains and production of functionally active, individual soluble HLA class II trimolecular complexes, wherein the soluble HLA class II trimolecular complexes comprise a soluble alpha chain, a soluble beta chain and an endogenously loaded peptide displayed in an antigen binding groove formed by the alpha and beta chains. The functionally active, soluble individual HLA class II trimolecular complex maintains the physical, functional and antigenic integrity of a native HLA trimolecular complex.

A primary application of the secreted class II product described herein is the screening of patients awaiting a transplant for anti-HLA antibodies. The requirement for an anti-HLA antibody screening assay is based on the observation that particular events (such as but not limited to, blood transfusion, bacterial infection, and pregnancy) cause one individual to produce antibodies directed against the HLA of other people (Bohmig et al., 2000; Emonds et al., 2000; and Howden et al., 2000). Such anti-HLA antibodies must be detected before a patient receives a transplant, or the transplanted organ will be immediately rejected. Thus, screening for anti-HLA class II antibodies is a prerequisite for organ transplantation.

All transplant patients (approximately 20,000 a year in the U.S.) and all those waiting for a transplant (more than 60,000 a year in the U.S.) must regularly (monthly is preferred) be screened for antibodies that target the HLA of other people. Therefore, these secreted or soluble HLA (sHLA) class II products provide native proteins for quickly and accurately identifying anti-HLA antibodies in those awaiting a transplant. This pre-transplant diagnostic test will prevent rapid organ failure.

The presently disclosed and claimed inventive concept(s) is further directed to a method of producing any of the anti-MHC removal devices described herein above or otherwise contemplated herein. In the method, a serologically active, soluble MHC moiety (as described herein above) is covalently coupled to a solid support (as described herein above). The soluble MHC moiety is attached to the solid support in such a manner that the soluble MHC moiety maintains the physical, functional and antigenic integrity of a native MHC trimolecular complex. In addition, the anti-MHC removal device is constructed so that a biological sample may be brought into contact with the device in a manner that allows the biological sample to interact with the soluble MHC moiety thereof, whereby anti-MHC antibodies specific for the MHC moiety attach to the soluble MHC moiety and are detected and/or removed from the biological sample. The method of producing the anti-MHC removal device may include any steps contemplated or otherwise described herein or otherwise known in the art.

The presently disclosed and claimed inventive concept(s) is further directed to a method for removing anti-MHC antibodies from a biological sample. Such antibody removal is useful, for example, when a patient attacks their transplanted organ with anti-HLA antibodies. Anti-HLA antibodies can also be removed prior to transplantation to enable better outcomes. The removal of antibodies specific for a particular HLA lessens the need for immune suppressing drugs. In the method for removing anti-MHC antibodies from a biological sample, an anti-MHC removal device as described herein above is provided. A biological sample is then brought into contact with the anti-MHC removal device, whereby at least a portion of the antibodies present in the biological sample that are specific for the serologically active, soluble MHC moiety (that is disposed on the surface of the anti-MHC removal device) are removed from the biological sample. The method may further include the step of recovering the biological sample following contact with the anti-MHC removal device, whereby the antibodies specific for the MHC moiety are substantially reduced in the recovered biological sample. For example, but not by way of limitation, the antibodies specific for the MHC moiety may be reduced by at least 25%, at least 30%, at least 40%, at least 50%, at least 60%, at least 70%, at least 80%, at least 90%, or at least 95% in the recovered biological sample; alternatively, the antibodies specific for the MHC moiety may be reduced by 20% to 95%, 25% to 95%, 30% to 90%, 40% to 85%, or 50% to 80% in the recovered biological sample. The method may further include repeating at least once the steps of contacting the biological sample to the anti-MHC removal device and recovering the biological sample following said contact. The use of multiple rounds of treatment provides an adequate reduction in antibody titers. In certain embodiments, the recovered biological sample may be substantially free of anti-MHC antibodies specific for the serologically active, soluble MHC moiety of the anti-MHC removal device.

When the anti-MHC removal device includes a device (such as a column) that possesses an inlet, an outlet, and a chamber disposed therebetween (with an inner surface on which the serologically active soluble MHC moiety is disposed), the biological sample is introduced into the chamber via the inlet. The biological sample is then allowed to flow through the device, and at least a portion of the anti-MHC antibodies specific for the serologically active MHC moiety attach thereto and are removed from the biological sample. The flow through is then collected from the outlet, whereby the presence of anti-MHC antibodies specific for the serologically active MHC moiety is substantially reduced.

When the anti-MHC removal device includes a human use device, the method may further include the step of placing the recovered biological sample back into a patient from which it was originally taken.

In certain additional embodiments, the method may further include the step of eluting the anti-MHC antibodies from the anti-MHC removal device. This step may be performed to allow for regeneration and reuse of the anti-MHC removal device. Alternatively, the eluted anti-MHC antibodies may be recovered and used as clinical agents. For example but not by way of limitation, the eluted, recovered anti-MHC antibodies may be utilized for quality control reagents for diagnostics and/or clinical proficiency testing. Thus, compositions that include the eluted, recovered anti-MHC antibodies are also encompassed by the scope of the presently disclosed and claimed inventive concept(s).

The presently disclosed and claimed inventive concept(s) further includes kits useful for removing anti-MHC antibodies from a biological sample. The kit may contain any of the devices described herein, and the kit may further contain other reagent(s) for conducting any of the particular methods described or otherwise contemplated herein. The nature of these additional reagent(s) will depend upon the particular assay format, and identification thereof is well within the skill of one of ordinary skill in the art. In addition, positive and/or negative controls may be included with the kit, and the kit may further include a set of written instructions explaining how to use the kit. The kit may further include a reagent (such as a competitive binding reagent) for elution of the anti-MHC antibodies from the device, thus allowing for regeneration and reuse thereof. Kits of this nature can be used in any of the methods described or otherwise contemplated herein.

### EXAMPLES

Examples are provided hereinbelow. However, the presently disclosed and claimed inventive concept(s) is to be understood to not be limited in its application to the specific experimentation, results and laboratory procedures. Rather, the Examples are simply provided as one of various embodiments and are meant to be exemplary, not exhaustive.

### Example 1

### Production of Class II sHLA Trimolecular Complexes for Use in Anti-MHC Removal Devices

This Example is directed to the expression of soluble individual human HLA class II trimolecular complexes in mammalian immortal cell lines. The method includes the use of modifications that alter the endogenous membrane bound complexes in such a way that the membrane bound anchor is disrupted, thereby allowing the cell to secrete the HLA class II trimolecular complexes. In this Example, the Alpha and Beta chain genes encoding HLA class II-DR, HLA-DQ, and HLA-DP were truncated such that the transmembrane and cytoplasmic domains were deleted. At the site of the truncation, a leucine zipper (a tethering moiety) replaced the transmembrane and cytoplasmic that endogenously anchors HLA to the membrane. The leucine zipper allows the HLA to be secreted from the cell while maintaining the class II trimolecular complex native confirmation (FIGS. 1 and 2). The leucine zipper is comprised of an acid segment tailing the class II alpha chain with complementary basic domain tailing the class II beta chain. The acid and basic segments fuse by means of the amino acid leucine being placed every 7 amino acids in the d position of the heptad repeat. The strategy was used by Chang in 1994 to bind the alpha and beta chains of soluble T cell Receptors together in the same fashion.

HLA class II complexes are comprised of two different polypeptide chains, designated α and β. In one method, the alpha and beta constructs were commercially purchased and directly ligated into a mammalian expression vector. In another, the constructs were produced by PCT amplification as described in the paragraph below, followed by purification and ligation into a mammalian expression vector.

Amplification of specific HLA class II genes from genomic DNA or cDNA was accomplished using PCR oligonucleotide primers for alleles at the HLA-DRα HLA-DRA), DRβ (HLA-DRB); DQα (DQA), DQβ (DQB); or DPα (DPA) and DPβ (DPB) gene loci. The beta chain 3′ PCR primer incorporates the leucine sequence consistent with the basic sequence of the leucine zipper dimer. The Alpha chain 3′ primer incorporates the leucine sequence consistent with the acid sequence of the leucine zipper dimer. The truncation of the class II genes through placement of the PCR primers eliminates the cytoplasmic and transmembrane regions, thus resulting in a soluble form of HLA class II trimolecular complex with a leucine zipper moiety.

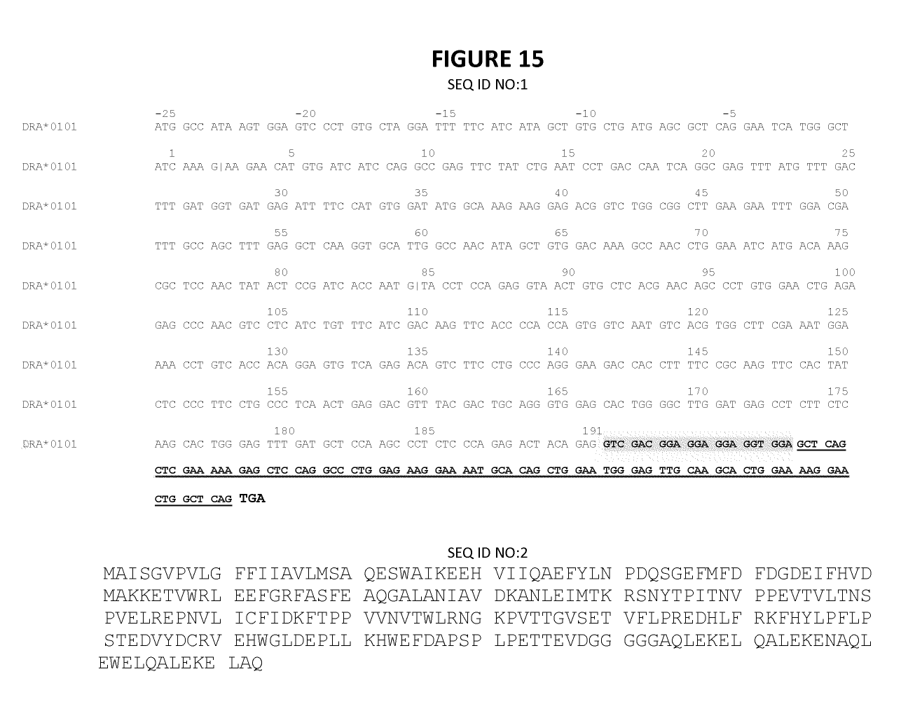

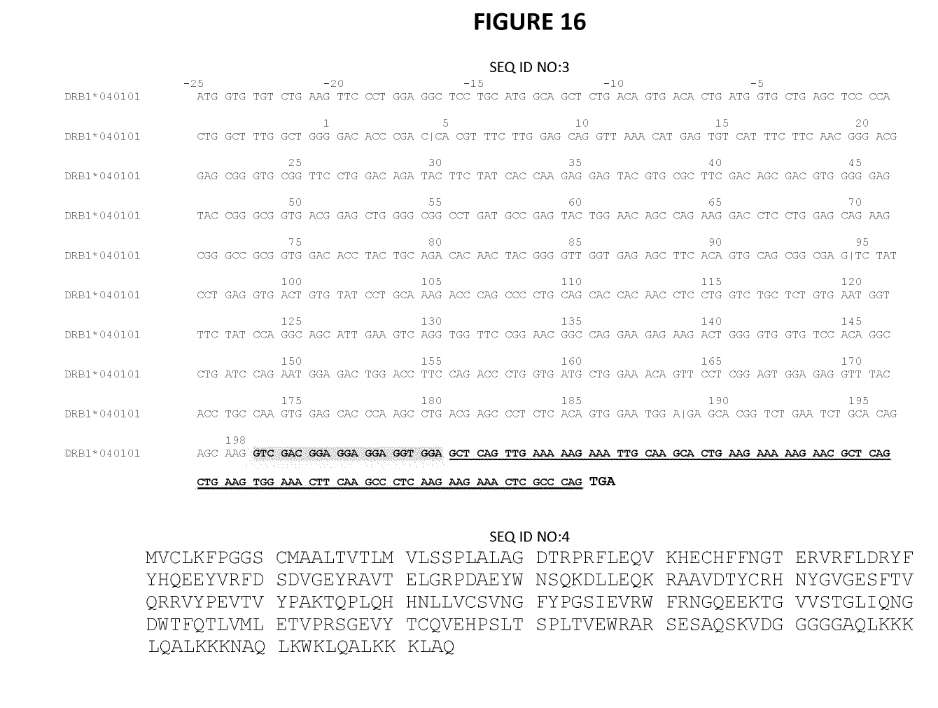

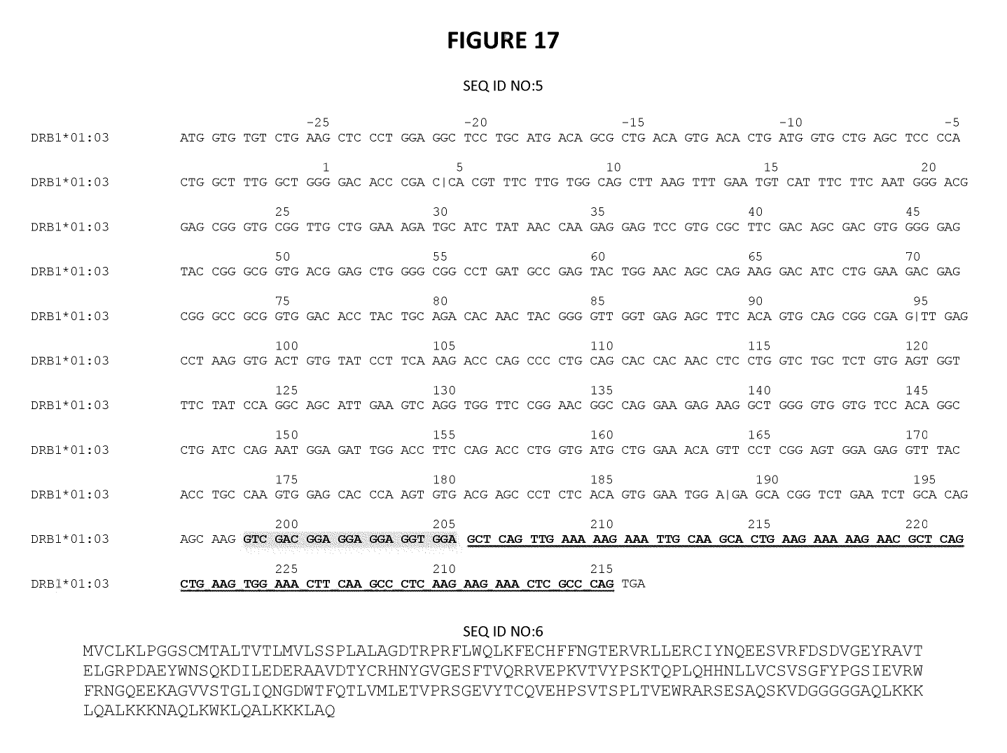

FIGS. 15-17 represent constructs used in the methods of sHLA production of the presently disclosed and claimed inventive concept(s). FIG. 15 illustrates the nucleic acid and amino acid sequences for a DRA1*0101 alpha chain-leucine zipper construct (SEQ ID NOS:1 and 2, respectively). FIG. 16 illustrates the nucleic acid and amino acid sequences for a DRB1*0401 beta chain-leucine zipper construct (SEQ ID NOS:3 and 4, respectively). FIG. 17 illustrates the nucleic acid and amino acid sequences for a DRB1*0103 beta chain-leucine zipper construct (SEQ ID NOS:5 and 6, respectively).

The constructs were then inserted into a mammalian expression vector. In one instance, the alpha chain was cut with one set of restriction enzymes, while the beta chain was cut with another set of restriction enzymes. The purified and cut alpha chain amplification products were ligated into the mammalian expression vector pcDNA3.1. Next, this ligated vector containing the sHLA class II alpha gene was transformed into E. coli strain JM109. The bacteria were grown on a solid medium containing an antibiotic to select for positive clones. Colonies from this plate were picked, grown and checked to contain insert. Plasmid DNA was isolated from the identified positive clones and subsequently DNA sequenced to insure the fidelity of the cloned alpha gene.

The alpha vector was re-cut using a second set of restriction enzymes which facilitate directional cloning of the purified beta PCR product. The final ligation product consisted of both alpha and beta clones. Plasmid DNA was then isolated from positive clones, and the beta genes were DNA sequenced from these clones.

Plasmid DNA for the alpha and beta class II alleles was prepared and DNA sequenced to confirm fidelity of the amplified class II genes. Log phase mammalian cells and the plasmid DNA were mixed in a plastic electrocuvette. This mixture was electroporated, placed on ice and resuspended in media. Special optimization was required for the electroporation step to enable successful enablement of the presently disclosed and claimed inventive concept(s). Standard electroporation procedures were unsuccessful in extensive trials by the inventors and as reported by other labs in publications.

After incubation for 2 days at 37° C. in a CO₂ incubator, the cells were subjected to selection with the antibiotic. First cells were counted and viability was determined. The cells were then resuspended in conditioned complete media. Next, cells were placed into each well of a 24-well plate and left to undergo selection. Supernatant from each well was taken, and an ELISA assay was performed to determine sHLA class II production. High producers were expanded and cryopreserved for large-scale production.

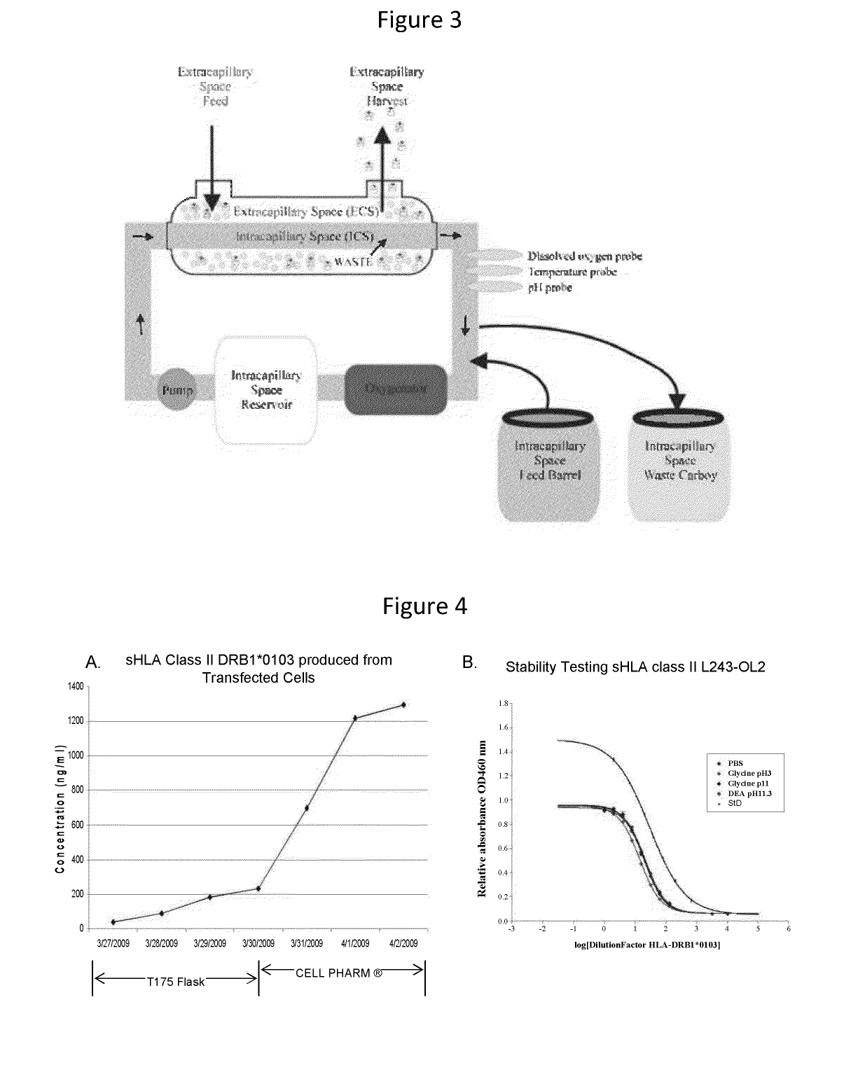

Prior to culture in CELL PHARM® bioreactors, the cellular growth parameters (pH, glucose, and serum supplementation) for each line was optimized for growth in bioreactors. Approximately 8 liters of naïve or pathogen infected sHLA-secreting class II transfectants were cultured in roller bottles in culture media supplemented with penicillin/streptomycin and serum or ITS (insulin-transferrin-selenium) supplement. The total volume of cells cultured was adjusted such that approximately 5×10⁹ cells were obtained. Cells were pelleted by centrifugation and resuspended in 300 ml of conditioned medium in a CELL PHARM® feed bottle. Cells and conditioned medium were inoculated through the ECS feed pump of a Unisyn CP2500 CELL PHARM® into 30 kDa molecular-weight cut-off hollow-fiber bioreactors previously primed with media supplemented with penicillin/streptomycin and serum or ITS. The culture of cells inside the CELL PHARM® was continued with constant monitoring of glucose, pH and infection. Medium feed rates were monitored and adjusted to maintain a glucose level of 70-110 mg/dL. FIG. 3 provides an overview of the CELL PHARM® bioreactor system; the sHLA secreting cells and their sHLA product were contained within the extra capillary space (ECS) of the hollow fiber bioreactor. Nutrients and gases for the cells were provided by recirculated medium.

FIG. 4A illustrates the increased production of sHLA class II DRB1*0103 produced from transfected cells when scaled up to the bioreactor production. The sHLA was purified from the cell supernatant with the specific anti-HLA class II antibody L243 coupled to CNBr-activated SEPHAROSE® 4B, and the protein concentration determined by a micro-BCA protein assay, UV absorbance and ELISA. The sHLA class II titer of a typical production run was found to be approximately 4-5 mg/liter of growth media. FIG. 4B illustrates that these trimolecular complexes were very stable in a wide variety of buffers and at a wide range of pH concentrations using monoclonal antibody L243, which reacts with virtually all DR HLA proteins. L243 is a murine IgG2a anti-HLA-DR monoclonal antibody previously described by Lampson & Levy (1980); said monoclonal antibody has been deposited at the American Type Culture Collection, Rockville, Md., under Accession number ATCC HB55.

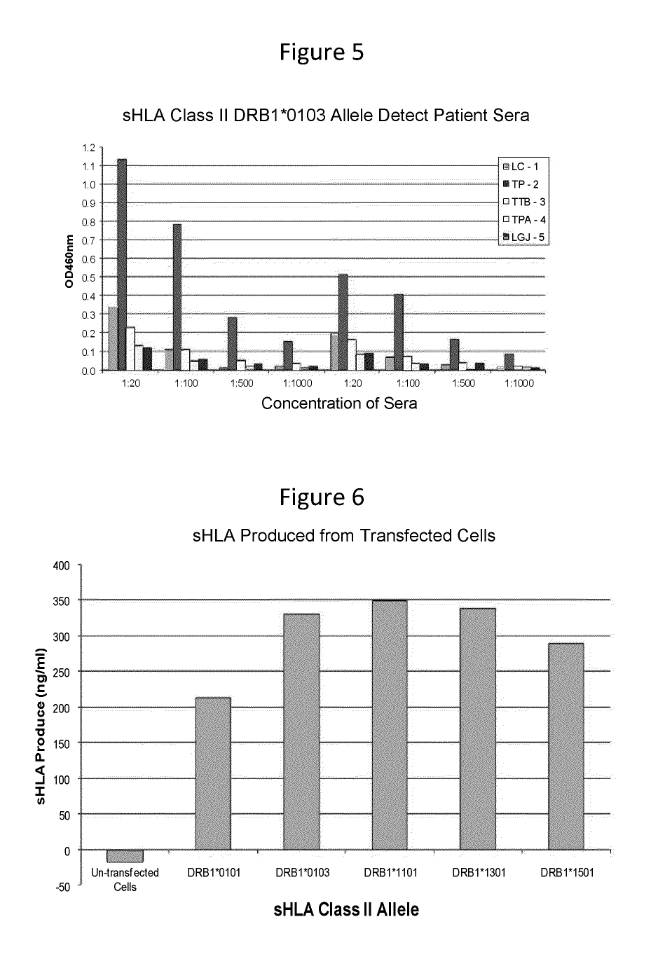

In FIG. 5, the serologic integrity of the purified sHLA class II trimolecular complexes was confirmed by directly coating the complexes on a plate and exposing the coated complexes to defined commercially available mAbs and patient sera. In addition, comparison of the sHLA with full-length molecules showed no differences in antigenicity.

FIG. 6 illustrates the ability to produce multiple different sHLA class II trimolecular complexes by the methods of the presently disclosed and claimed inventive concept(s). While DRB1*0101, DRB1*0103, DRB1*1101, DRB1*1301 and DRB1*1501 are shown for the purposes of example, multiple other sHLA class II trimolecular complexes have also been produced in milligram quantities in accordance with the presently disclosed and claimed inventive concept(s). Trimolecular complexes from each sHLA DR protein have been detected and quantitated using the L243 ELISA-based assay.

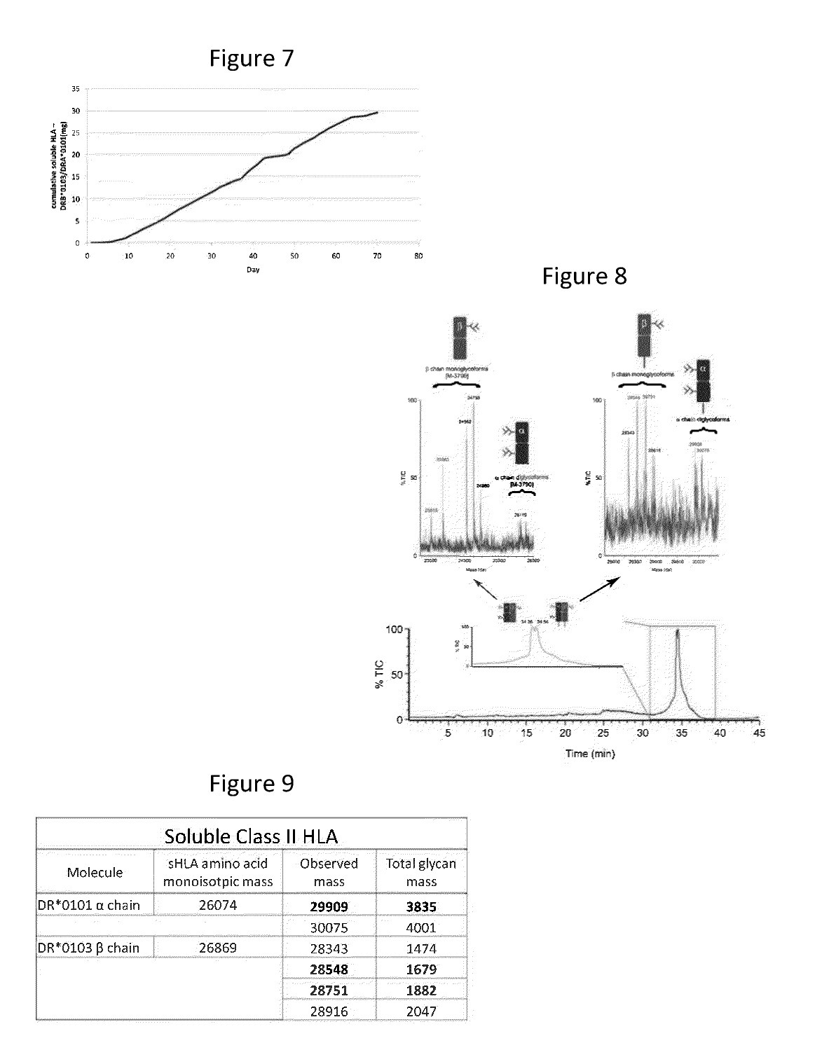

FIGS. 7-9 illustrate another example of sHLA class II production in accordance with the presently disclosed and claimed inventive concept(s). In this example, immortalized cells transfected with a soluble HLA-DRB*0103/DRA*0101 construct (DRB1*0101 soluble alpha chain with leucine zipper and DRB1*0103 soluble beta chain with leucine zipper) were grown in a roller bottle format until a total 1¹⁰ cells were obtained. The cells were then seeded into the ECS portion of 2 hollow fiber bioreactor units. Cells were grown in DMEM+10% FBS in the ECS and no FBS in the ICS. ECS harvest was collected every day until cells were dead and no longer producing soluble HLA. Protein was quantified using a capture ELISA. For this ELISA an antibody specific for the leucine zipper (2H11) was used as the capture antibody, and an antibody specific for class II HLA (L243) as the detector antibody. Approximately 8 mg of soluble HLA was loaded on an affinity antibody (L243) column and eluted in an alkaline buffer (0.1M Glycine, pH 11). Fractions containing soluble HLA were pooled and lyophilized. The lyophilate was resuspended in water/20% acetonitrile and loaded onto a C18 RP-HPLC column. The soluble HLA was then eluted using a 20% to 80% acetonitrile gradient and detected using electrospray ionization TOF mass spectrometry.

As can be seen in FIG. 7, milligram quantities of a soluble form of a single class II HLA heterodimer were produced in the bioreactor format. Additionally, the intact heterodimer was purified with no other contaminating proteins, as determined by LCMS (FIG. 9). This soluble class II contains a monoglycosylated beta chain and diglycosylated alpha consistent with native class II HLA (FIG. 8). Furthermore, the various glycoforms were consistent with the natural variation in sugars that occurs as a protein transits to the cell surface. For a subpopulation of the class II molecules, intracellular proteolytic events removed all but two amino acids of the leucine zipper domain from both the alpha and the beta chains. However, like the full length construct, class II without the leucine zipper domain remain as a heterodimer as both the alpha and beta chains co-elute. These soluble class I and class II HLA proteins are amenable to analysis by mass spectrometry, whereby the purity and identity of these proteins can be confirmed by TOF analysis of molecular weights (FIG. 9).

### Example 2

### Use of Class II sHLA for Antibody Removal

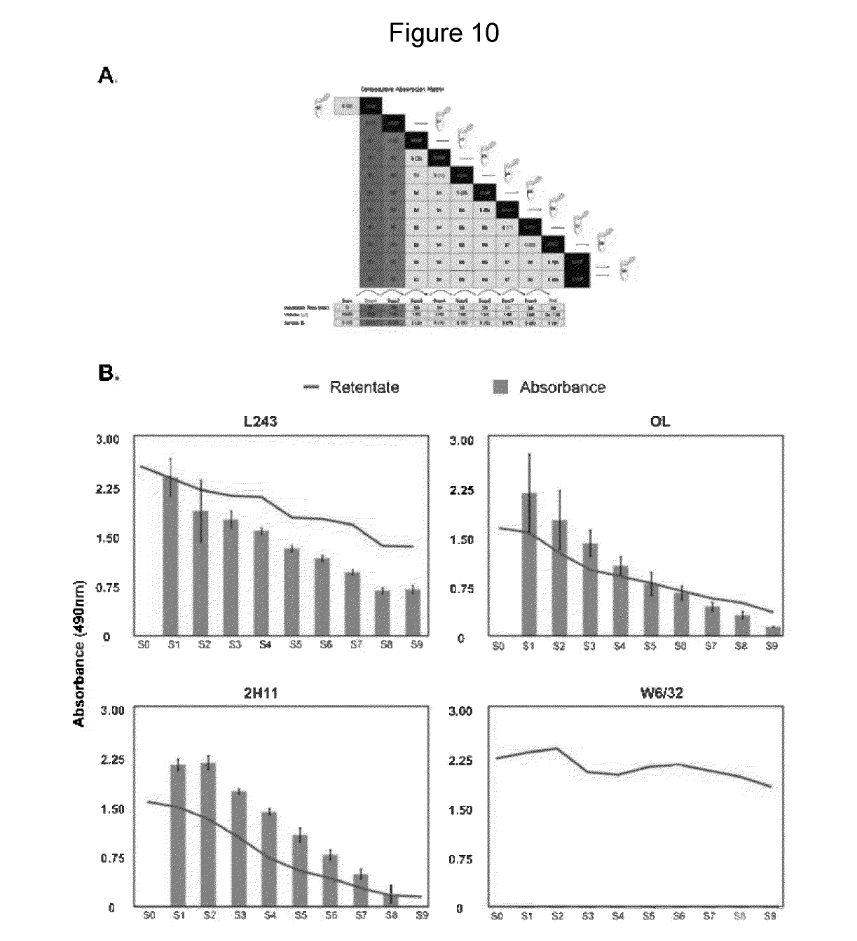

The soluble HLA class II trimolecular complexes of the presently disclosed and claimed inventive concept(s) have also been demonstrated herein to be successfully used in antibody removal techniques, as illustrated in FIGS. 10-14.

FIG. 10 graphically depicts coupling of soluble DRB1*1101 ZP HLA Class II trimolecular complex to a solid support and use thereof to facilitate removal of HLA Class II specific antibodies in an ELISA format. Panel A contains a diagram of the consecutive absorption matrix ELISA performed for specific antibody removal. Briefly, soluble HLA Class II DRB1*1101/DRA1*0101 ZP (labeled as DRB1*1101) was coated to a standard ELISA plate and blocked with BSA. Biotinylated labeled HLAII specific antibodies were then prepared and diluted according to a pre-determined titration for optimal binding, and added to 10 wells as S1. A small portion of this original dilution (200 μl) was saved as S(0). The antibody was allowed to bind for 30 minutes at room temperature, after which the entire contents of each well (<200 μl) was moved to a corresponding new well (S2), and BSA buffer was added to the S1 wells. This entire process was repeated for a total of 9 sample rounds (S1-S9). For each round, one well was saved in an eppendorf tube for evaluation of the amount of antibody remaining in the retentate solution. These were marked as S(n). After the absorption process was completed, the plate was developed using HRP/OPD peroxidase substrate and plotted as “absorbance.” The retentate samples were also read on a separate ELISA plate in the same manner. These were plotted as “retentate.” Panel B depicts absorbance and retentate values from 3 different HLA Class II specific mAb antibodies: L243, OL (One Lambda), and 2H11 were subjected to the consecutive absorbance matrix. The L243 and OL mAbs, specific for the HLA Class II molecules, and the 2H11 mAb, specific for the zipper tail piece recombinantly added to the soluble HLA Class II molecules, showed a reduction of HLA class II antibodies in the absorption and retentate through each round of the ELISA. One control mAb antibody was included, W6/32, which is specific for HLA Class I molecules, which was not absorbed to the plate and only found in the retentate.

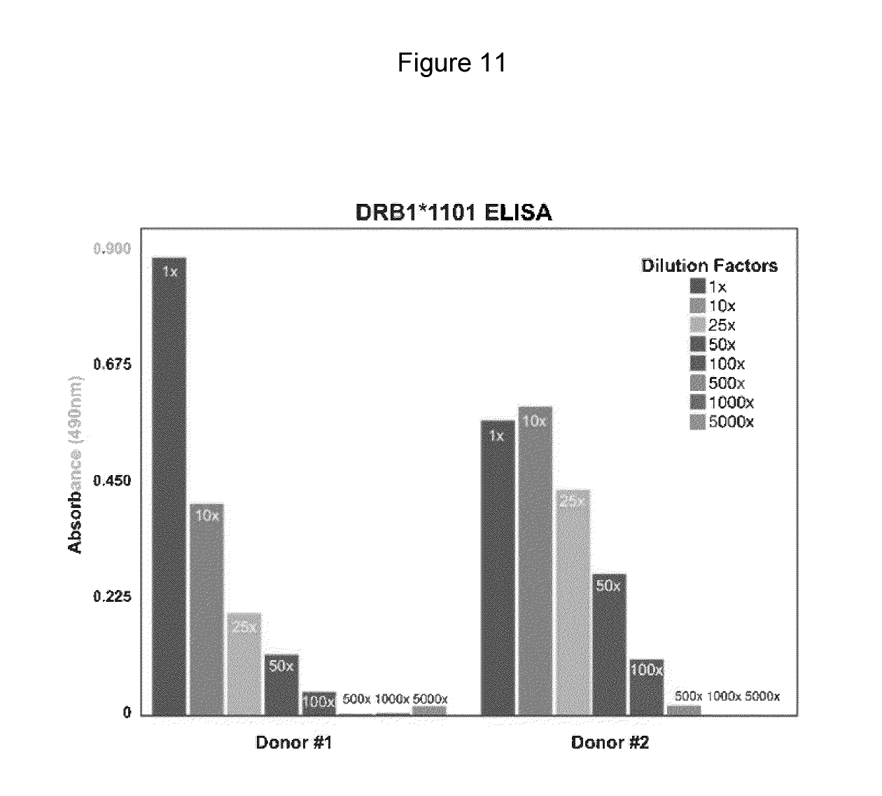

FIG. 11 graphically depicts that DRB1*1101-specific human sera was recognized by soluble DRB1*1101 in an ELISA format. Using soluble HLA Class II DRB1*1101/DRA1*0101 ZP (labeled as DRB1*1101), ELISA plates were directly coated with the HLA Class II soluble allele. Serum samples from two human donors known previously to have DRB1*1101 reactivity were added to the plates in a dilution range from 1× (no dilution) to 5000×. Plates were washed, and a secondary biotinylated goat anti-human IgG antibody was added. Plates were developed using HRP/OPD peroxidase substrate and read at absorbance of 490 nm. Dilution curves for the sera antibody reactivity can be seen for both donors, corresponding to specific avidity for DRB1*1101.

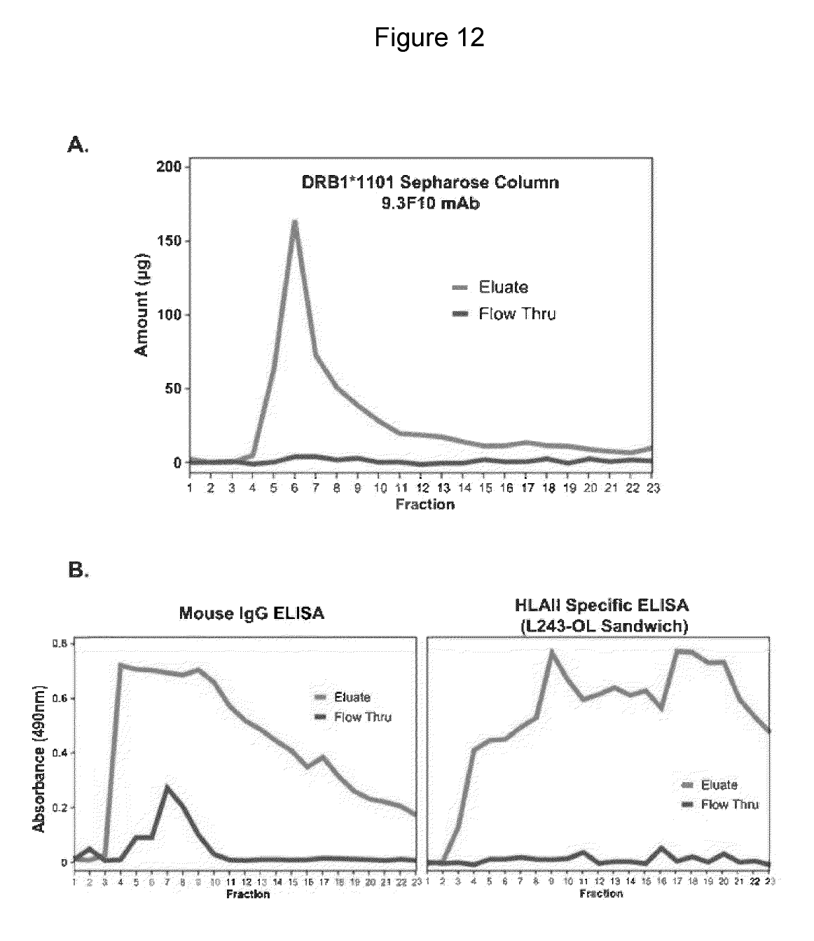

FIG. 12 graphically depicts that soluble DRB1*1101 can be coupled to SEPHAROSE® and used to absorb the HLA Class II specific antibody, 9.3F10. In Panel A, 4 mg of soluble DRB1*1101 was coupled to 1 ml of SEPHAROSE® Fast Flow and packed into a gravity column. A known mixture of 100 μg/ml of mAb 9.3F10 (in 1×PBS), which has DR reactivity, was passed over the column and washed with 1×PBS. A total of 23 200 μl fractions of flow thru were collected, weighed, and measured for OD 280 nm. Values were converted to total amount of protein. To elute the column, roughly 4 ml of DEA (diethanolamine) buffer, pH 11.3, was added to the column, and fractions were collected in 200 μl quantities. The eluate was also weighed, measured at an optical density of 280 nm, and converted to total amount of protein.

In Panel B of FIG. 12, two separate ELISAs for total mouse IgG and human HLA were also performed on the Flow Thru and Eluate to detect specific antibodies (versus HLA proteins) that might have been eluted off the column. Due to the increase in ELISA sensitivity, the minuscule amount of protein seen in the flow thru gave a small peak in the antibody ELISA. Importantly, however, no HLA was seen in the flow thru, but HLA did elute off the column when DEA was added.

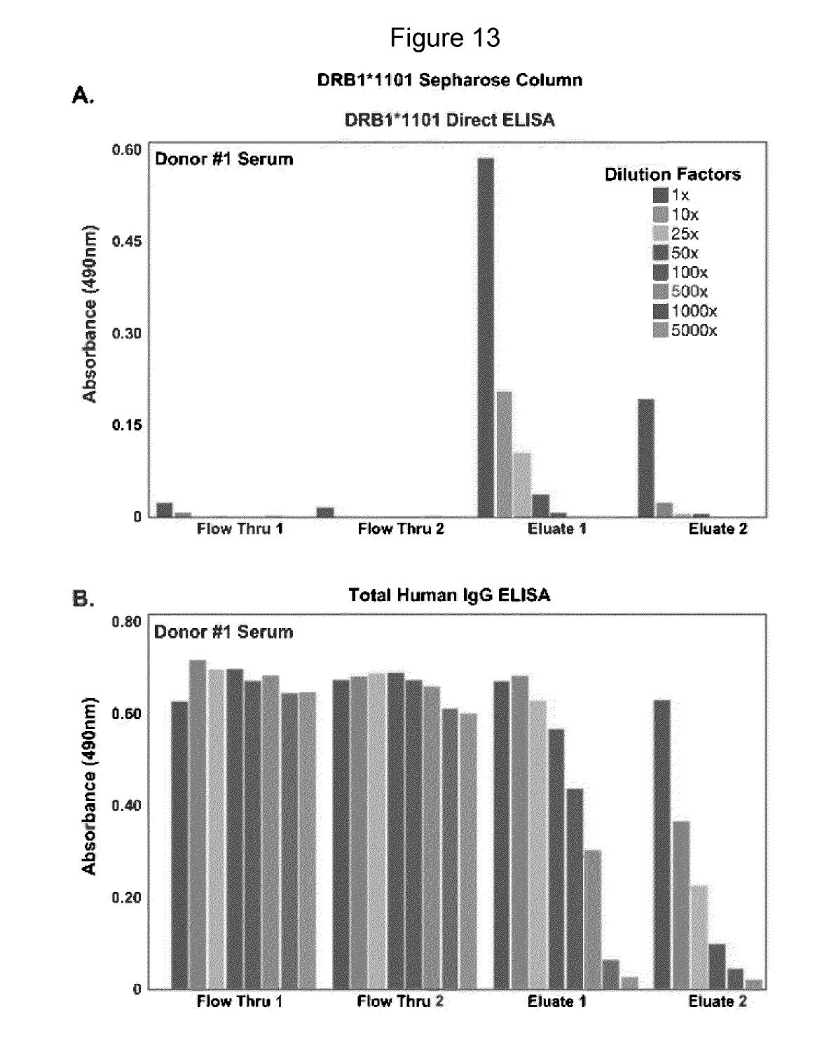

FIG. 13 graphically depicts that antibodies contained in human sera specific for DRB1*1101 can be removed by a DRB1*1101 specific column. Donor #1 sera was passed over the DRB1*1101 SEPHAROSE® column, and two 2 ml fractions of flow thru were collected. To elute, DEA buffer, pH 11.3 was added to the column, and two 2 ml fractions were collected. In Panel A, a direct DRB1*1101 ELISA was performed to detect the amount of DRB1*1101 specific antibodies that were left in the flow thru and eluate. Flow thru and eluate fractions were diluted 1× (no dilution) to 5000× and developed with a biotinylated goat anti-human secondary antibody, followed by HRP/OPD peroxidase substrate. Plates were read at an optical density of 490 nm. In Panel B, a total human IgG sandwich ELISA was also performed to evaluate passage of total human IgG. Total human IgG was seen to pass thru; however only DRB1*1101 antibodies were retained by the column, and only seen once the column was eluted.

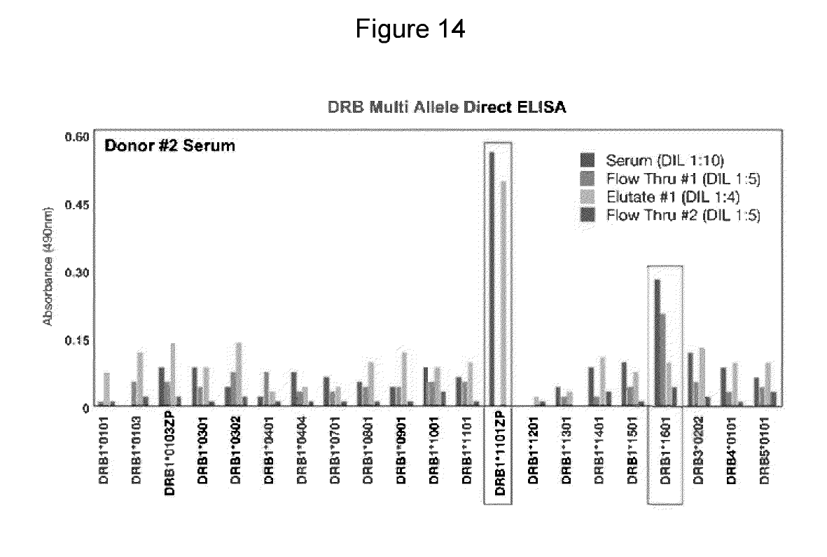

FIG. 14 graphically depicts that soluble DRB1*1101 coupled SEPHAROSE® is specific for DRB1*1101 and not other DR alleles. Donor #2 sera was passed over the same DR1*1101 column in the same manner as FIG. 13, and two fractions of the flow thru and one fraction of the eluate were evaluated for multi-allele DR reactivity. Briefly, multiple alleles of DR from membrane detergent purifications and two DR alleles produced solubly were coated to a 96 well ELISA plate in previously determined optimal amounts for reactivity. Two flow thru fractions and one of the eluate fractions were compared to the original sera sample for reactivity. The second eluate fraction was not evaluated given that most of the specific reactivity was contained in Eluate #1 (FIG. 14). Low reactivity was seen across the board except for the soluble DRB1*1101 (DRB1*1101 ZP) allele, which gave high reactivity to only the sera sample and the eluate but not the flow thrus (first boxed area). The sera also contained strongly reactive antibodies to a second allele, DRB1*1601 (second boxed area), which passed through the flow thru but not the eluate.

Therefore, this Example demonstrates that sHLA class II trimolecular complexes immobilized in a column format can selectively and efficiently remove the vast majority of anti-HLA specific antibodies based on affinity to the bound HLA class II protein in a single pass through, while not removing antibodies that bind to antigenically dissimilar HLA molecules. These data show that a highly specific and efficient antibody removal device can be constructed using the sHLA class II proteins produced in accordance with the presently disclosed and claimed inventive concept(s).

### Example 3

### Isolation of HLA-DR11 Antibodies from Sensitized Human Sera

To test the hypothesis that antigen-based isolation of naturally occurring, polyclonal, anti-HLA antibodies would facilitate the characterization of allogeneic anti-HLA antibody responses, appreciable quantities of soluble class II HLA molecules were produced in a native conformation. Next, this unique HLA reagent was used to construct the first reported HLA immunoaffinity column. Donor sera containing a complex mixture of anti-HLA antibodies were then passed over the column. Antibodies specific for a particular class II HLA were retained on the column, and these immunoglobulins were subsequently recovered by elution and characterized. The phenotypic and functional profiling of antigen-specific antibodies represents a substantial advance in the ability to understand how anti-HLA antibodies contribute to organ rejection. A robust application of this technology would distinguish complement-fixing antibodies that represent a contraindication for transplantation from refractory humoral responses that are less of a concern. These immunoaffinity columns constructed with native soluble HLA might also provide a new generation of therapeutic tools for patients with strong antibody reactivity directed towards allogeneic HLA.

### Materials and Methods of Example 3

Patient serum samples: Donor 1 serum was purchased as HLA-DR11 antiserum (Gen-Probe, Inc., San Diego, Calif.). Donor 2 serum was collected from a DR11 sensitized kidney recipient using informed consent according to a protocol approved by the University of Texas Southwestern institutional review board. Donor 2, a 50 year old male, received a kidney graft with a 6/6 mismatch (graft HLA: A2, A3, B62, B51, DR4, DR11). After transplantation donor 2 rejected the graft and developed anti-HLA antibodies. Approximately 5 ml of whole blood was collected and allowed to coagulate. The blood was then centrifuged and the serum was removed from the pellet. Sera were stored at 4° C. until testing.

sHLA-DR11 Protein Production. To produce secreted HLA-DRB11 (sHLA) molecules, α-chain cDNAs of HLA-DRA1*01:01 and HLA-DRB1*11:01 were modified by PCR mutagenesis to delete codons encoding the transmembrane and cytoplasmic domains and add the leucine zipper domains. For DRA*01:01, a 7 amino acid linker (DVGGGGG; SEQ ID NO:7) followed by leucine zipper ACIDp1 was added. For DRB*11:01 the same linker was used, followed by leucine zipper BASEp1 sequence (Busch et al., 2002). sHLA-DRA1*01:01 and sHLA-DRB1*11:01 were cloned into the mammalian expression vector pcDNA3.1(−) geneticin and zeocin respectively (Invitrogen, Life Technologies, Grand Island, N.Y.). The HLA class II deficient B-LCL cell line NS1 (ATCC # TIB-18) was transfected by electroporation simultaneously with sHLA-DRB1*11:01 and DRA1*01:01. Two days post-electroporation cells were transferred into selective growth media containing G418 (0.8 mg/ml) and zeocin (1 mg/ml). Drug resistant stable transfectants were tested for production of sHLA class II molecules by sandwich ELISA using L243 (Leinco Technologies Inc., St. Louis, Mo.) as a capture and class II specific commercial antibody for detection (One Lambda Class II, One Lambda Inc., Canoga Park, Calif.). Individual wells with clonal cell populations were tested for the production of sHLA class II by ELISA and the highest producing clone was expanded in an ACUSYST-MAXIMIZER® hollow fiber bioreactor (Biovest International, Inc., Minneapolis, Minn.). Approximately 25 mg of sHLA-DR11 was harvested from each bioreactor. sHLA-DR11 containing supernatant was loaded on a L243 immunoafffinity column and washed with 40 column volumes of 20 mM phosphate buffer, pH 7.4. sHLA-DR11 molecules were eluted from the affinity column with 50 mM DEA at pH 11.3, neutralized with 1M TRIS pH 7.0, and buffer exchanged and stored at 1 mg/ml in sterile PBS.

Mass spectrometry: 10 μg of purified sHLA-DR11 was reduced and denatured with dithiothreitol (Sigma-Aldrich D0632) and incubated at 95° C. for 5 minutes. The sample was then alkylated with iodoacetamide (Thermo Scientific 89671F), for 1 hour at room temperature. Denatured protein was digested with trypsin using a standard two step digestion protocol (Thermo Scientific 90055). Tryptic peptides were reconstituted in 30% acetic acid/70% ultra-pure water, and loaded onto the ULTIMATE® 3000 HPLC system (Dionex, Thermo Fisher Scientific, Inc., Sunnyvale, Calif.) with a PEPMAP™100 C18 75 μm×15 cm, 3 μm 100 Å reverse phase column. Peptides were eluted and analyzed on a QTOF QSTAR® Elite mass spectrometer (ABI, Thermo Fisher Scientific, MDS Sciex) with Mascot software.

Antibody Removal with DRB1*11:01-Coupled SEPHAROSE® Affinity Columns. For a 1 ml sHLA-DR11 affinity column, SEPHAROSE® 4 Fast Flow (GE Healthcare) was swollen and washed 4 times with ice-cold 1 mM HCl pH 3.0. The swollen matrix was mixed with sHLA-DR11 (4 mg) in bicarbonate coupling solution at a final reaction concentration of 1.6 mg/ml. After the reaction, the matrix was washed three times in coupling buffer and blocked with 0.1M TRIS, pH 8.0. The coupled matrix was then packed into a small 2 ml column.

Either 1 ml of a 200 μg/ml L243 antibody solution or 1 ml of total human sera was applied to the matrix and allowed to be absorbed by gravity. After sample application, 4 ml of PBS pH 7.4 was added. During this loading step, 25 fractions were collected manually, each containing ˜200 μl. Finally, the column was eluted by applying 5 ml of 50 mM DEA pH 11.3. 20 fractions were collected in the elution process and immediately neutralized with 35 μl of 1 M TRIS. For L243, collected fractions were measured by OD₂₈₀ for antibody content. After each procedure, column was mock eluted with DEA, pH11.3 followed by 50 ml of wash buffer (PBS pH 7.4).

Class II HLA Single Antigen Bead Assay and Ig Isotyping. Specificities of anti-HLA antibodies in the pre-column serum, flow through, and eluate were determined using a LUMINEX®-based class II HLA single antigen assay (Gen-Probe GTI Diagnostics), according to manufacturer protocols. Briefly, 40 μl of the bead suspension was incubated with 10 μl of the test sample at room temperature for 30 minutes. Beads were washed and incubated with the detecting antibody at room temperature for 30 minutes, then washed and analyzed on a LUMINEX® 100 analyzer. Data were analyzed using MATCHIT® (Gen-Probe GTI Diagnostics, San Diego, Calif.) and EXCEL® (Microsoft) software. Data for the starting sera and flow through are shown as background corrected median florescence intensity (BCMFI) values based on company defined background levels, which are lot specific and determined by a standard negative sera. With the eluate, there was substantially less background so the background was defined as the minimum bead MFI. For the flow through, the BCMFI values were normalized to the average DQ BCMFI in the starting sera (Tables 1 and 2). The eluate BCMFI values were normalized to the DRB1*11:01 BCMFI in the starting sera (Tables 1 and 2).

For antibody isotyping and quantification the BIO-PLEX PRO™ immunoglobulin isotyping kit (Bio-Rad Laboratories, Inc., Hercules, Calif.) was used according to manufacturer protocols. Briefly, 10-fold serial dilutions of the sample were made and 50 μl of the sample was incubated with 50 μl of the bead suspension for 30 minutes at room temperature. Beads were washed and incubated with the detecting antibody at room temperature for 30 minutes. Last, beads were washed and analyzed on a LUMINEX® 100 (One Lambda, Inc.). Sample MFI values were translated into Ig concentration using the Ig specific standard curves.

Complement Dependant Cytolysis. Complement dependant cytolysis (CDC) was determined using the Lambda Cell Tray: 30 B cell panel (One Lambda, Inc.) Cell lines analyzed were DR11 positive. Cell line class II HLA haplotypes are as follows. C433: DR4, DR11, DR52, DR53, DQ7. C418: DR4, DR11, DR52, DR53, DQ7. C423: DR11, DR13, DR52, DQ6, DQ7. C428: DR11, DR17, DR52, DQ2, DQ7 (One Lambda, Inc.). Lysis was performed on indicated samples according to manufacturer protocols. Rabbit complement was used as a source of complement. After lysis, FLOROQUENCH™ dye (One Lambda, Inc.) was used to differentiate live cells from lysed cells. Live cells and lysed cells were then analyzed using a Nikon TE200-E florescent microscope. Whole well images were generated for each well using the 4× objective lens for both the green filter (excitation: 490 nm bp 20, emission: 520 nm bp 38) and the red filter (excitation: 555 nm bp 28, emission: 617 nm bp 73). Total florescence in both channels was determined using MetaMorph v 7.5.5.0 and percent cell death was calculated as red florescence/red florescence+green florescence.

### Results for Example 3

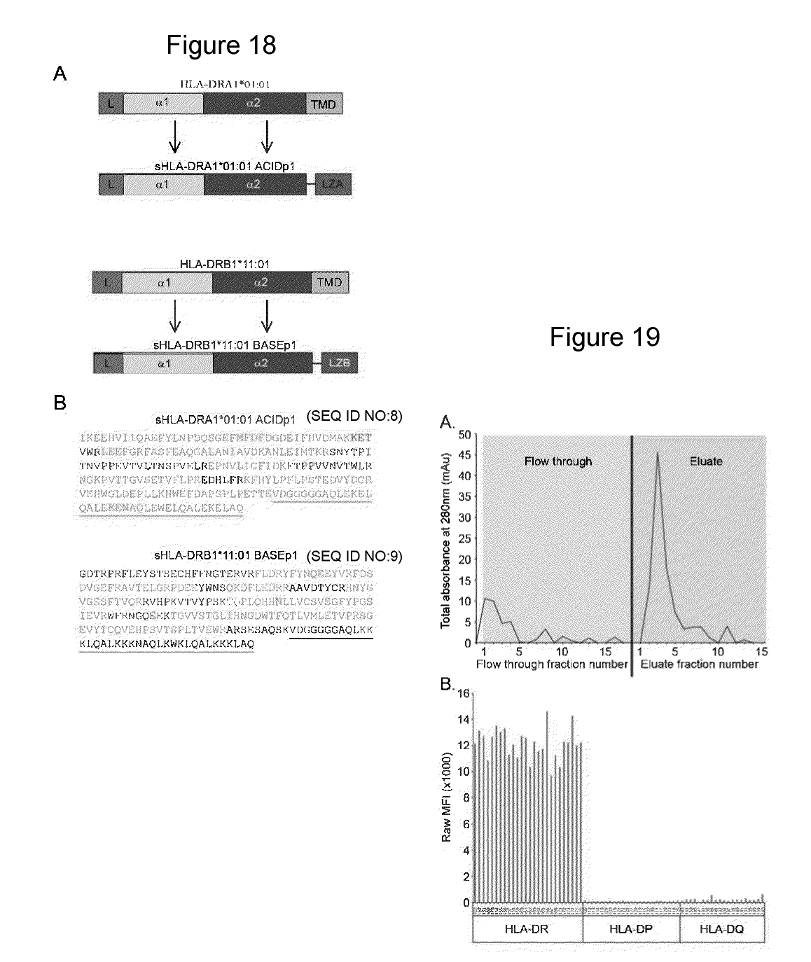

Production and Purification of Soluble Class II HLA. The specific isolation of anti-class II HLA antibodies requires a source of plentiful, native class II HLA. While there are several techniques for obtaining HLA proteins, in this Example, soluble molecules were produced in mammalian cells because these HLA are glycosylated, naturally loaded with ligands, and fully reactive with antibodies. One challenge is that HLA class II exists as an alpha/beta heterodimer and these proteins must be specifically paired to be functional. Previous studies have stabilized the class II soluble HLA heterodimer by replacing the transmembrane and cytoplasmic domains on both the alpha and beta chains with complementary leucine zipper domains (Busch et al., 2002; and Kalandadze et al., 1996), but these studies have only succeeded using non-mammalian cells. Here this approach was used to generate constructs for HLA-DRA1*01:01 and HLA-DRB1*11:01, in which the transmembrane domain is replaced with a 7 amino acid linker followed by an ACIDp1 or BASEp1 leucine zipper domain respectively (FIG. 18A).

A murine cell line was chosen for sHLA-DR11 production, because the inventors hypothesized that the endogenous mouse class II MHC alpha and beta proteins (H2-A^{d}, H2-E^{d}) would not pair with the soluble human class II HLA alpha and beta proteins nor interfere with the intended pairing of the soluble alpha/beta HLA proteins. To confirm that the purified sHLA-DR11 was free from mouse alpha and beta chains, the purified protein was digested with trypsin, and the resulting peptides were subjected to liquid chromatography mass spectrometry (LCMS) analysis. In a BLAST analysis, the peptide sequences showed no matches with the endogenous mouse class II MHC(H2-A^{d}, H2-E^{d}), while peptide sequences were detected from both the alpha and beta chains of the sHLA-DR11 construct transfected into the cells (FIG. 18B). Thus, it was concluded that the desired alpha and beta chain of sDR11 was produced and purified without contamination from other class II MHC subunits.

Column Removal of Anti-HLA Class II Antibodies. In order to test sHLA class II in an immunoaffinity column format, sHLA-DR11 was purified and coupled to CNBr activated SEPHAROSE® 4 Fast Flow solid support matrix. The anti-HLA-DR monoclonal antibody L243 was passed over the affinity column to test whether the sHLA-DR11 complexes remained intact during the coupling process and to measure the binding capacity of the column. Fractions of 200 μl were collected during the loading process (flow through), and bound L243 was eluted intact. Between the flow through and the eluate, 78% (170.6 μg) of the antibody loaded onto the column was recovered, of which 28% (47.8 μg) was in the flow through and 72% (122.9 μg) in the eluate (FIG. 19A). Furthermore the captured and eluted L243 antibody maintained its HLA-DR binding activity and specificity (FIG. 19B). These results demonstrated that HLA-DR11 retained its native conformation when coupled to the affinity column matrix and that a sHLA-DR11 column could be used to remove and recover intact anti-HLA antibodies.

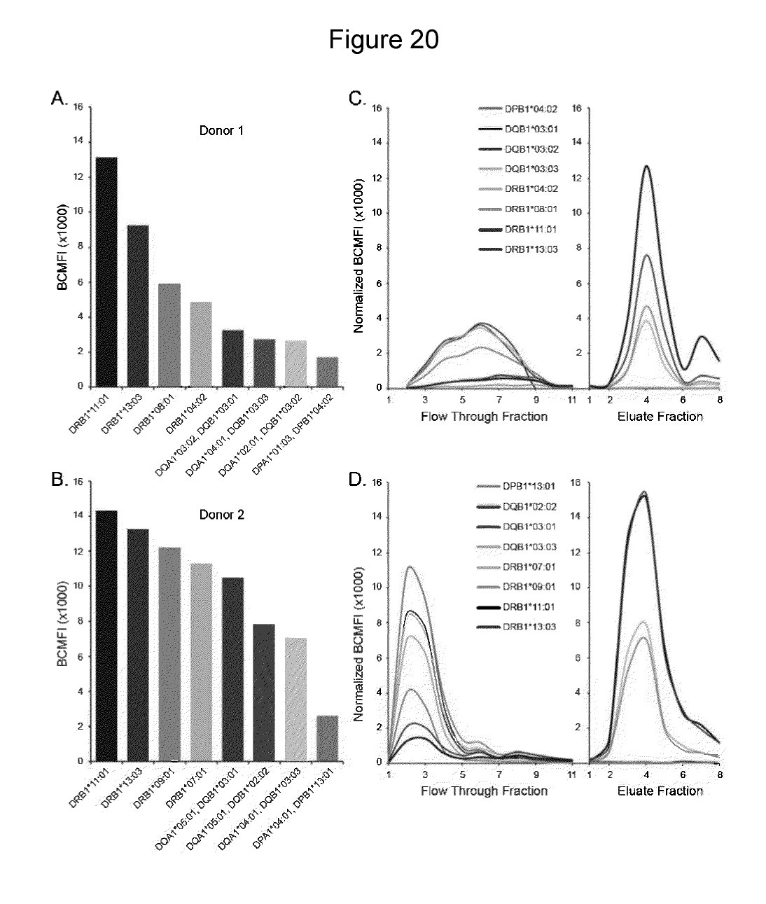

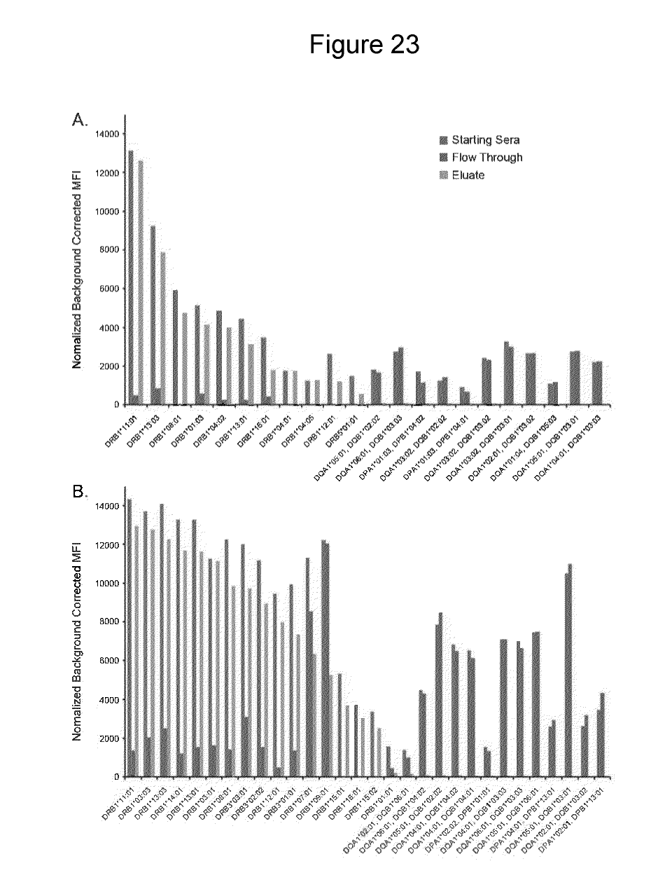

Depletion and Recovery of Anti-HLA-DR11 Antibodies from Patient Sera. Nest, it was tested whether the column could be used to deplete anti-HLA-DR11 antibodies from patient sera. Sera from two DR11 sensitized patients were analyzed for reactivity to multiple class II HLA types in the starting serum (prior to column loading), flow-through, and eluate. Both starting sera contained complex mixtures of polyclonal anti-HLA antibodies reactive with multiple DR and DQ specificities (FIGS. 20A and B). Following passage through the DR11 column, the flow through and eluate from each donor were quite distinct in their patterns of HLA reactivity (FIGS. 20C and D). In the donor 1 serum, HLA-DQ (red) and -DP (green) specific antibodies flowed through the column, while the majority of antibodies to DR11, 13, 8, and 4 were depleted from the serum and subsequently recovered in the eluate. Likewise, in the donor 2 serum, HLA-DQ and -DP specific antibodies passed through the column. However, unlike the donor 1 serum, the majority of DR9 and DR7 specific antibodies from the donor 2 serum flowed through the column, while antibodies to DR11 and DR13 were retained and subsequently eluted. Only small amounts of DR9 and DR7 specific antibodies were recovered in the eluate. All class II HLA reactivity in the starting sera, pooled flow-through (fractions 2-11), and pooled eluate (fractions 2-6) is summarized in FIG. 23.

Prior to column passage, these sera recognized a substantial number of DR specificities (11 HLA-DR in donor 1 and 17 HLA-DR in donor 2). Strikingly, the DR11 column depleted 100% (11/11) of the DR reactive antibodies in donor 1 and 88% (15/17) in donor 2 (FIG. 23, Tables 1 and 2), while no HLA-DQ or DP reactive antibodies were recovered. Thus, the DR11 column removed antibodies to multiple serologically related HLA-DR specificities while antibodies reactive to HLA-DQ and -DP did not bind. These results show that DR11 specific antibodies can be depleted and recovered from patient sera while antibodies reactive with other antigens are not retained.

TABLE 1

| | Pre | Flow Through | | Eluate | |
|---|---|---|---|---|---|
| Bead | Sera | | Normalized* | | Normalized^{†} |
| Antigens | BCMFI | BCMFI | BCMFI | MFI | MFI |
| DRB1*11:01 | 13136 | 517 | 498 | 12063 | 12619 |
| DRB1*13:03 | 9245 | 890 | 857 | 7530 | 7877 |
| DRB1*08:01 | 5945 | 49 | 47 | 4563 | 4773 |
| DRB1*01:03 | 5140 | 612 | 589 | 3964 | 4147 |
| DRB1*04:02 | 4890 | 290 | 279 | 3857 | 4035 |
| DRB1*13:01 | 4447 | 280 | 270 | 2999 | 3137 |
| DRB1*16:01 | 3477 | 456 | 439 | 1705 | 1784 |
| DRB1*04:01 | 1767 | 0 | 0 | 1694 | 1772 |
| DRB1*04:05 | 1243 | 0 | 0 | 1257 | 1315 |
| DRB1*12:01 | 2632 | 0 | 0 | 1172 | 1225 |
| DRB5*01:01 | 1496 | 0 | 0 | 574 | 600 |
| DQA1*05:01, | 1813 | 1728 | 1663 | 97 | 101 |
| DQB1*02:02 | | | | | |
| DQA1*06:01, | 2743 | 3084 | 2969 | 88 | 92 |
| DQB1*03:03 | | | | | |
| DPA1*01:03, | 1729 | 1201 | 1156 | 82 | 85 |
| DPB1*04:02 | | | | | |
| DQA1*03:02, | 1245 | 1472 | 1417 | 75 | 78 |
| DQB1*02:02 | | | | | |
| DPA1*01:03, | 907 | 713 | 686 | 75 | 78 |
| DPB1*04:01 | | | | | |
| DQA1*03:02, | 2422 | 2436 | 2344 | 65 | 68 |
| DQB1*03:02 | | | | | |
| DQA1*03:02, | 3273 | 3109 | 2992 | 51 | 53 |
| DQB1*03:01 | | | | | |
| DQA1*02:01, | 2674 | 2780 | 2676 | 43 | 45 |
| DQB1*03:02 | | | | | |
| DQA1*01:04, | 1113 | 1224 | 1178 | 36 | 38 |
| DQB1*05:03 | | | | | |
| DQA1*05:01, | 2745 | 2891 | 2783 | 25 | 26 |
| DQB1*03:01 | | | | | |
| DQA1*04:01, | 2229 | 2322 | 2235 | 24 | 25 |
| DQB1*03:03 | | | | | |
| Normalization | | | 0.96 | | 1.05 |
| Ratio | | | | | |

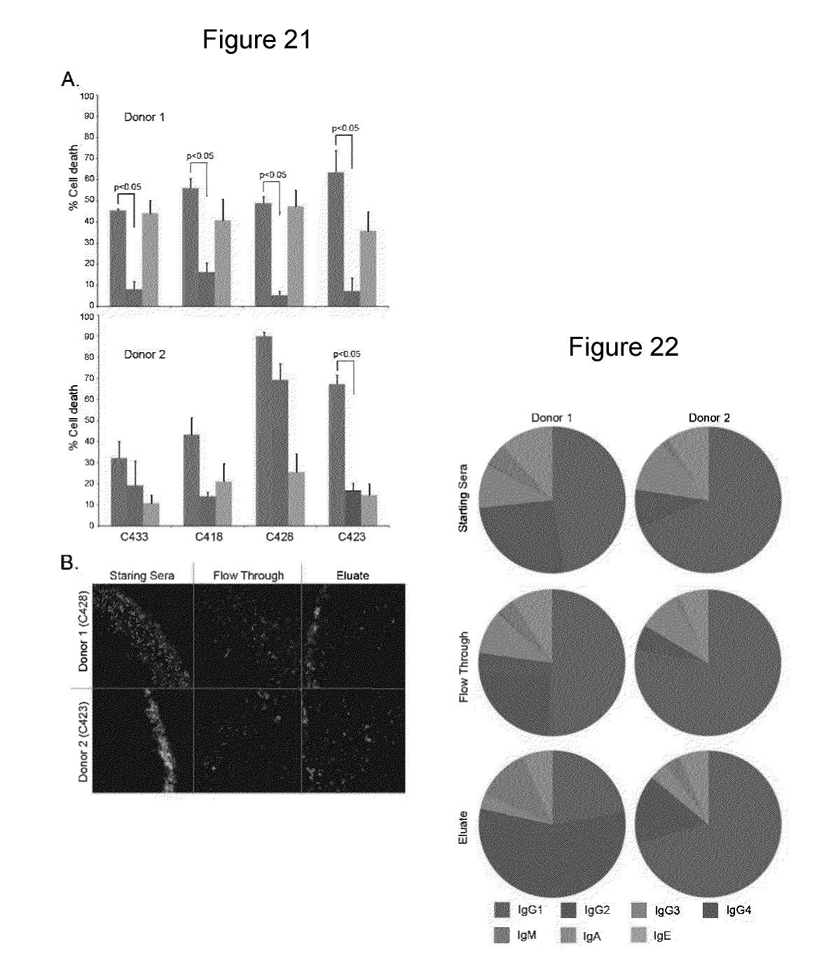

Background corrected MFI values for Donor 1 used to generate FIG. 21A and FIG. 23.
*BCMFI values was normalized to the average DQ BCMFI in the starting sera.
^{†}BCMFI values was normalized to the DRB1*11:01 BCMFI in the starting sera.

TABLE 2

| | Pre | Flow Through | | Eluate | |
|---|---|---|---|---|---|
| Bead | Sera | | Normalized* | | Normalized^{†} |
| Antigens | BCMFI | BCMFI | BCMFI | MFI | MFI |
| DRB1*11:01 | 14320 | 1094 | 1386 | 10721 | 12934 |
| DRB1*03:03 | 13703 | 1618 | 2050 | 10567 | 12748 |
| DRB1*13:03 | 14101 | 1980 | 2509 | 10146 | 12241 |
| DRB1*14:01 | 13267 | 973 | 1232 | 9667 | 11663 |
| DRB1*13:01 | 13268 | 1233 | 1563 | 9622 | 11608 |
| DRB1*03:01 | 11249 | 1285 | 1628 | 9232 | 11138 |
| DRB1*08:01 | 12247 | 1130 | 1431 | 8150 | 9832 |
| DRB3*03:01 | 12014 | 2434 | 3085 | 8065 | 9730 |
| DRB3*02:02 | 11172 | 1216 | 1541 | 7399 | 8926 |
| DRB1*12:01 | 9453 | 390 | 494 | 6599 | 7961 |
| DRB3*01:01 | 9915 | 1073 | 1360 | 6078 | 7333 |
| DRB1*07:01 | 11299 | 6741 | 8543 | 5247 | 6330 |
| DRB1*09:01 | 12218 | 9504 | 12044 | 4370 | 5272 |
| DRB1*15:01 | 5333 | 0 | 0 | 3092 | 3730 |
| DRB1*16:01 | 3729 | 0 | 0 | 2530 | 3052 |
| DRB1*15:02 | 3374 | 0 | 0 | 2076 | 2505 |
| DRB1*01:01 | 1569 | 362 | 459 | 180 | 217 |
| DQA1*02:01, | 1391 | 787 | 997 | 130 | 156 |
| DQB1*06:01 | | | | | |
| DQA1*06:01, | 4460 | 3376 | 4278 | 93 | 112 |
| DQB1*04:02 | | | | | |
| DQA1*05:01, | 7845 | 6681 | 8467 | 90 | 108 |
| DQB1*02:02 | | | | | |
| DQA1*04:01, | 6815 | 5123 | 6492 | 76 | 92 |
| DQB1*04:02 | | | | | |
| DQA1*04:01, | 6534 | 4833 | 6125 | 71 | 86 |
| DQB1*04:01 | | | | | |
| DPA1*02:02, | 1566 | 1049 | 1329 | 69 | 83 |
| DPB1*01:01 | | | | | |
| DQA1*04:01, | 7082 | 5599.5 | 7096 | 67 | 81 |
| DQB1*03:03 | | | | | |
| DQA1*06:01, | 7024 | 5252 | 6656 | 63 | 75 |
| DQB1*03:03 | | | | | |
| DQA1*05:01, | 7463 | 5899.5 | 7476 | 59 | 71 |
| DQB1*06:01 | | | | | |
| DPA1*04:01, | 2607 | 2319 | 2939 | 30 | 36 |
| DPB1*13:01 | | | | | |
| DQA1*05:01, | 10486 | 8673 | 10991 | 28 | 34 |
| DQB1*03:01 | | | | | |
| DQA1*02:01, | 2645 | 2499 | 3167 | 26 | 31 |
| DQB1*03:02 | | | | | |
| DPA1*02:01, | 3463 | 3402 | 4311 | 21 | 25 |
| DPB1*13:01 | | | | | |
| Normalization | | | 1.27 | | 1.21 |
| Ratio | | | | | |

Background corrected MFI values for Donor 2 used to generate FIG. 21B and FIG. 23.
*BCMFI values was normalized to the average DQ BCMFI in the starting sera.
^{†}BCMFI values was normalized to the DRB1*11:01 BCMFI in the starting sera.

Purified HLA-DR11 Antibodies Fix Complement. To evaluate the functional traits of antibodies for HLA-DR11, the complement fixing activity of the donor 1 and donor 2 starting sera, flow through, and eluate were tested. HLA-DR11 positive cells were incubated with starting sera, flow through, or eluate in the presence of complement. Complement dependent cytolysis (CDC) was measured with florescent microscopy. In donor 1 serum, the DR11 column depleted CDC activity to all 4 DR11 target cell types (FIG. 21; C433, C418, C428, C423), and this DR11 specific CDC activity was recovered in the eluate. Thus, anti-DR11 antibodies were necessary and sufficient for CDC activity in patient 1. The donor 2 serum showed heterogeneous reactivity to the different target cell lines in the assay. On some cell lines (C433, C418, C428), the removal of DR11 antibodies did not significantly reduce the CDC activity in the flow through, likely due to complement fixing antibodies directed towards the other HLA present on the target cells. Interestingly, CDC activity on cell line C423 was depleted in both the donor 2 flow through and eluate, indicating that anti-DR11 antibodies were necessary but not sufficient for CDC activity. These data demonstrate that antibodies to individual HLA can be isolated and functionally characterize, and that anti-HLA CDC activity can vary between individuals.

Quantity and Quality of Polyclonal HLA-DR11 Antibodies. The HLA immunoaffinity column provided a unique opportunity to study patient-derived populations of DR11 reactive antibodies. Antibody function is largely dictated by Ig constant region, or antibody isotype. Therefore, the isotype of DR11 reactive antibodies was characterized in patient sera. Several different isotypes were observed in the starting sera, the pooled flow through, and the pooled eluate for both donors (FIG. 22). IgG1 predominated in both the starting sera and in the flow through, with appreciable IgG2, IgA, IgG3, and some IgM present. The isotype profile of antibodies eluted from the DR11 column was diverse in both individuals, with 5 of the 7 Ig isotypes represented in the eluate. In the donor 1 eluate, IgG2 was the most common isotype, with considerable levels of IgG1, IgM, and IgA. In contrast, IgG1 predominated in the donor 2 eluate, with appreciable IgG2 and detectable IgA, IgM, and IgG3. The antibodies in the donor 1 eluate were 56.1% IgG2, 22.3% IgG1 and 11.6% IgM, whereas the donor 2 eluate contained 70.5% IgG1, 15.5% IgG2, and 3.3% IgM (FIG. 22). Both eluate samples showed similar low levels of IgA and IgG3, with IgA comprising 6.6% and 6.3% and IgG3 comprising 3.3% and 4.2% of the eluate for donor 1 and donor 2, respectively. This preliminary dataset suggests substantial heterogeneity can exist in anti-HLA antibody isotype.

The column depleted all detectable anti-HLA-DR activity from the donor 1 serum, allowing the total concentration of anti-HLA-DR antibodies in this patient to be estimated. The pooled eluate of donor 1 contained 17.7 μg/ml of antibody. Assuming the efficiency of antibody recovery from serum was similar to that of mAb L243, and factoring for volume variation, the serum concentration of the anti-HLA-DR antibody in donor 1 was approximately 23.7 μg/ml, or 0.05% of the total Ig. While this may not be representative of concentrations in other donor sera, it demonstrates that these immunoaffinity columns enable, for the first time, the direct quantification of anti-HLA antibodies in patient sera.

### Discussion of Example 3

Donor specific anti-HLA antibodies represent a pre-transplant contraindication and a post-transplant risk for graft loss. While it is clear that antibodies to HLA mediate graft failure and loss, studies also suggest that not all anti-HLA antibodies are detrimental (Wasowska, 2010; and Amico et al., 2009). These observations have sparked great interest in discerning what differentiates pathogenic anti-HLA antibodies from those that are not a threat to transplanted organs. To date, the tools available for studying antibodies to HLA have not been sufficient for characterizing or detecting antibodies that warrant clinical intervention. In this Example, HLA-DR11 immunoaffinity columns were used to characterize patterns of HLA-DR serologic cross-reactivity, to phenotype DR11 reactive antibodies, and to assess the function of antibodies in patient sera. This ability to isolate anti-HLA antibodies is positioned to augment both clinical and basic scientific endeavors by unraveling the complex nature of humoral responses to HLA.

Anti-HLA antibody responses are recognized as polyclonal and heterogeneous. In particular, allogeneic antibody responses to class II HLA are highly cross-reactive, with any given serum reacting to multiple class II HLA (EI-Awar et al., 2007). Indeed, antibodies reactive to the HLA-DR11 column recognized a striking diversity of HLA-DR specificities. The HLA-DR11 column depleted 11 different HLA-DR specificities from the donor 1 serum while 15 HLA-DR specificities were removed from donor 2 (FIG. 23). The HLA-DR11 reactive antibodies purified from donor 1 then reacted with HLA-DR103, 4, 8, 12, 13, 16, and 51 with no reactivity to the remaining 26 DR complexes tested. The pattern of serologic cross reactivity observed for donor 1 was consistent with the recognition of a solvent accessible Asp residue present at position 70 in the beta chain of all recognized HLA-DR complexes but in none of the other HLA-DR complex except HLA-DR7 (EI-Awar et al., 2007). The serologic reactivity pattern for antibodies recovered from donor 2 was more complex; the anti-HLA-DR11 antibodies cross reacted with every HLA-DR tested except for HLA-DR1, 103, 4, 10, 51, and 53. Interestingly, antibodies directed toward HLA-DR7 and HLA-DR9 were split into two groups; those that bound HLA-DR11 and those that did not (FIG. 23). This demonstrates the availability of two (or more) distinct epitopes in the HLA-DR7 and HLA-DR9 reactive antibody pool, only one of which is shared with DR11. These data illustrate the use of HLA immunoaffinity columns to characterize the target epitopes and cross-reactivity of anti-HLA antibodies, and the variability of anti-HLA reactivity profiles from patient to patient.

In addition to deciphering patterns of serologic recognition, HLA-DR11 reactive sera were analyzed for their isotype profile and ability to fix complement. The straightforward relationship between isotype profile and CDC activity in Donor 1 indicated that complement-fixing anti-HLA-DR11 antibodies (i.e., IgG1 and IgM) were responsible for anti-HLA-DR11 CDC activity in the Donor 1 starting serum and that the HLA-DR11 column removed complement fixing activity from the flow through by depleting HLA-DR11-reactive antibodies. The relationship between isotype profile and CDC activity was more complex for Donor 2. The Donor 2 eluate was dominated by non-complement-fixing IgG1, and CDC activity was lost in both the flow-through and eluate. This finding is consistent with antibody synergy, which has been previously described in complement fixation. Murine models of MHC class I mismatch during cardiac transplantation demonstrated that modest amounts of complement-fixing (IgG2a) antibodies to MHC fix complement much more effectively when combined with non-complement-fixing (IgG1) antibodies to MHC (Wasowska, 2010; and Murata et al. 2007). Thus, the CDC activity in the Donor 2 starting serum could have resulted from a combination of anti-HLA-DR11 IgG1 and complement-fixing antibodies without specificity for HLA-DR11, while the HLA-DR11 column eliminated HLA-DR11-elicited CDC activity from both the flow-through and eluate by separating these syngergistic antibodies. These results show that HLA immunoaffinity columns absorb complement-fixing activity in a sera-specific manner.

A long-term objective in the development of an HLA immunoaffinity matrix is antibody absorption. Antibody reduction therapies such as plasma exchange are now used for bulk antibody depletion to facilitate transplants for recipients who are otherwise serologically incompatible. One drawback to these existing reduction therapies is their lack of specificity, which results in the removal of beneficial as well as deleterious anti-HLA antibodies (Schmaldienst et al., 2001). The ability to specifically deplete anti-HLA antibodies could significantly improve existing immune reduction therapies. Antigen-specific antibody depletion columns are currently in use to remove antibodies specific for blood group A and B antigens (Crew et al., 2010; and Takahashi, 2007). While the column and serum volumes tested here were on a small scale, this column could be scaled up, similar to the columns for blood group antigens, in order to reduce anti-HLA-antibodies from patient plasma before or after transplantation.

In summary, an approach for producing milligram quantities of native class II HLA proteins in mammalian cells has been developed, and in this Example, these proteins have been successfully coupled to a column support used to purify anti-HLA antibodies. The DR11 reactive antibodies recovered were functionally intact and highly cross-reactive. Antibodies that recognized DR11 fixed complement in one of the two donors, and isotype profiles were consistent with CDC activity. These observations demonstrate that HLA immunoaffinity columns, or perhaps other platforms such as HLA coated magnetic beads, will provide transplant physicians and their supporting clinical HLA laboratories with the means to parse anti-HLA reactivity into acceptable or unacceptable categories on the basis of CDC activity, isotype profile, and serologic cross-reactivity with other HLA. HLA technologies like this antibody separation device will help elucidate which antibodies promote rejection. Lastly, these results establish the feasibility of using HLA immunoaffinity columns to study anti-HLA immunity and to achieve specific immune reduction for organ transplantation.

### Example 4

### SHARC (sHLA Antibody Removal Column) Analysis

Coupling CNBr (Cyanogen Bromide) vs NHS (N-Hydroxysuccinimide)

There are three primary properties of a matrix that indicate the effectiveness of the SHARC. These properties are: (1) coupling efficiency—the ability of an activated matrix to covalently link sHLA to the solid support; (2) binding capacity—the maximum quantity of antibody depleted by the sHLA linked matrix; and (3) regeneration efficiency—the number of times the matrix can be loaded and eluted (regenerated).

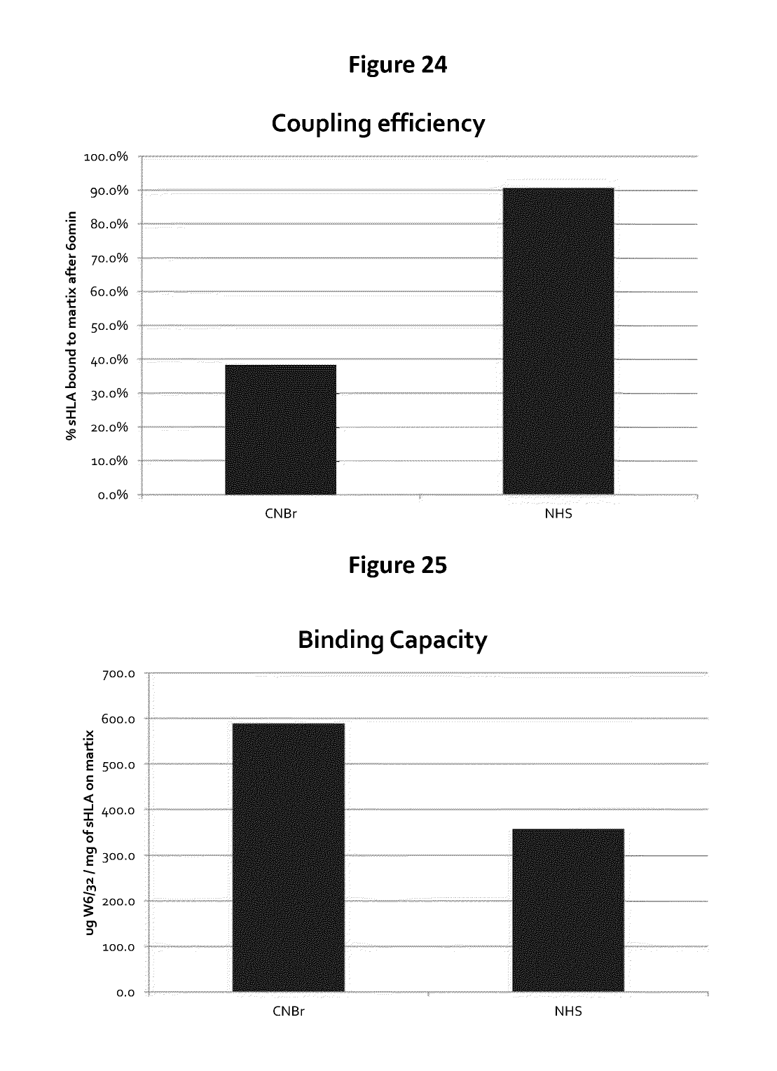

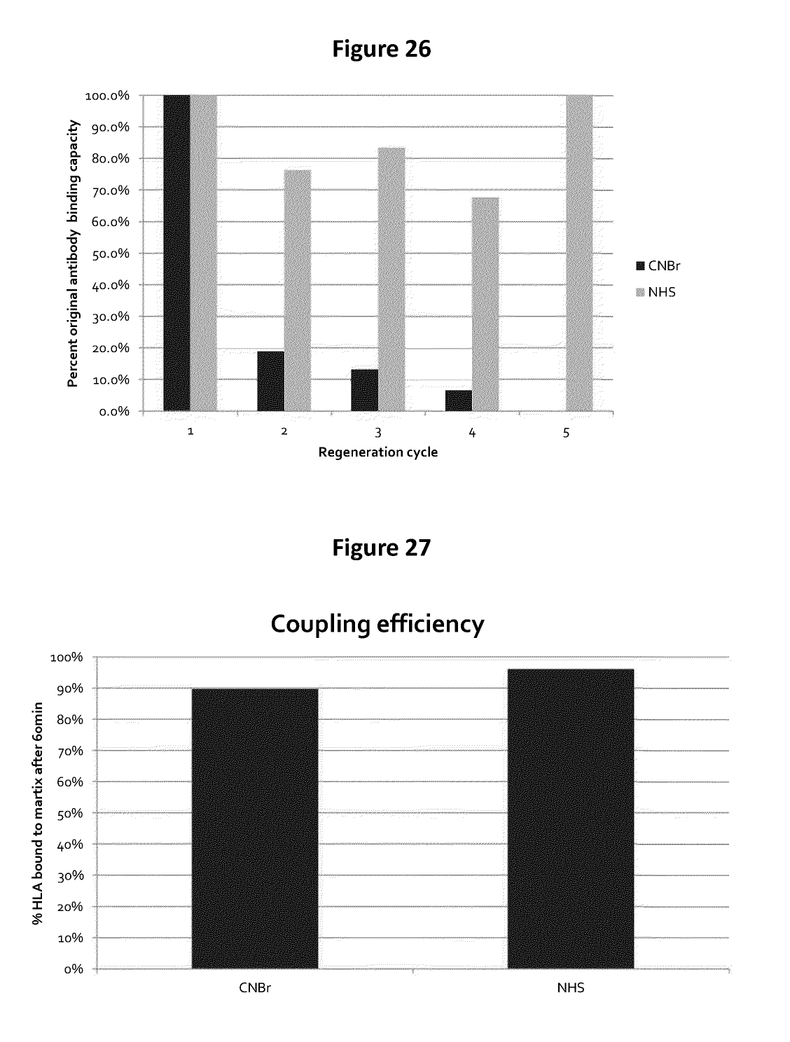

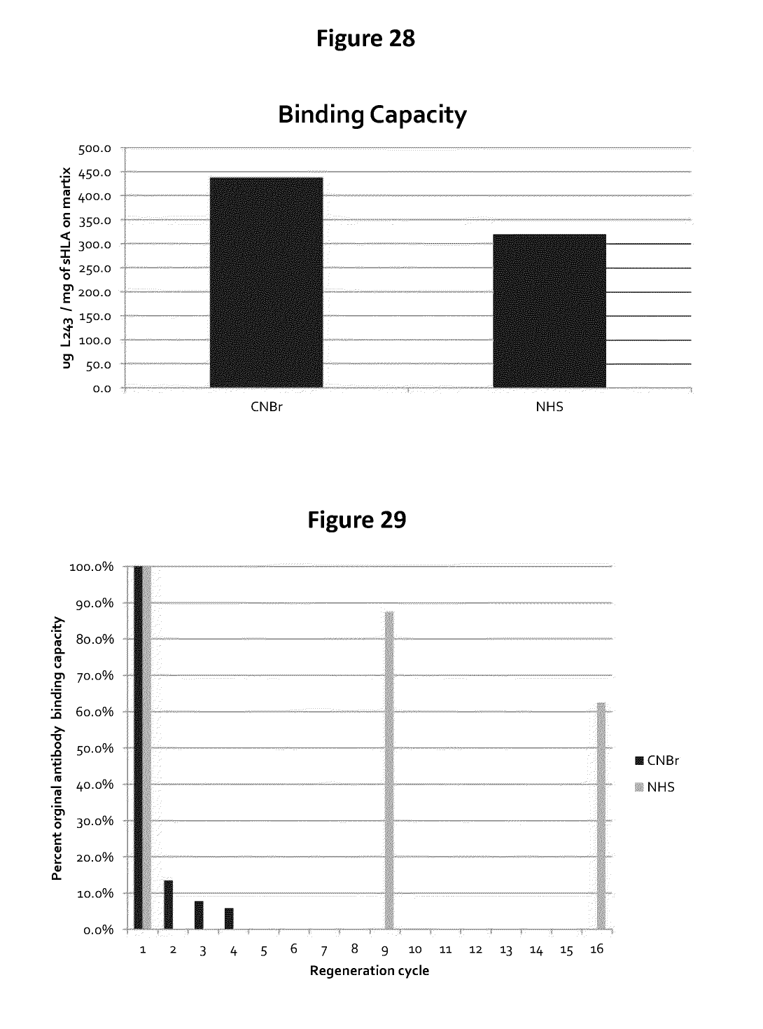

sHLA can be covalently linked to a solid support such as SEPHAROSE® using a number of different chemistries. In this Example, the aforementioned parameters were tested with either a CNBr- or NHS-SEPHAROSE® based chemistry to link sHLA to a SEPHAROSE® 4 fast flow matrix. Both CNBr and NHS chemistries were tested using class I and class II sHLA. In the case of class I sHLA, the NHS-based chemistry outperformed in both coupling efficiency (FIG. 24) and regeneration efficiency (FIG. 25); however, it exhibited a lower binding capacity (FIG. 25). For class II sHLA, coupling efficiencies were similar between NHS and CNBr (FIG. 27), but the binding capacity was higher with the CNBr matrix (FIG. 28); in addition, the regeneration efficiency was higher with the NHS matrix.

Full Scale Class I and Class II HLA SHARC

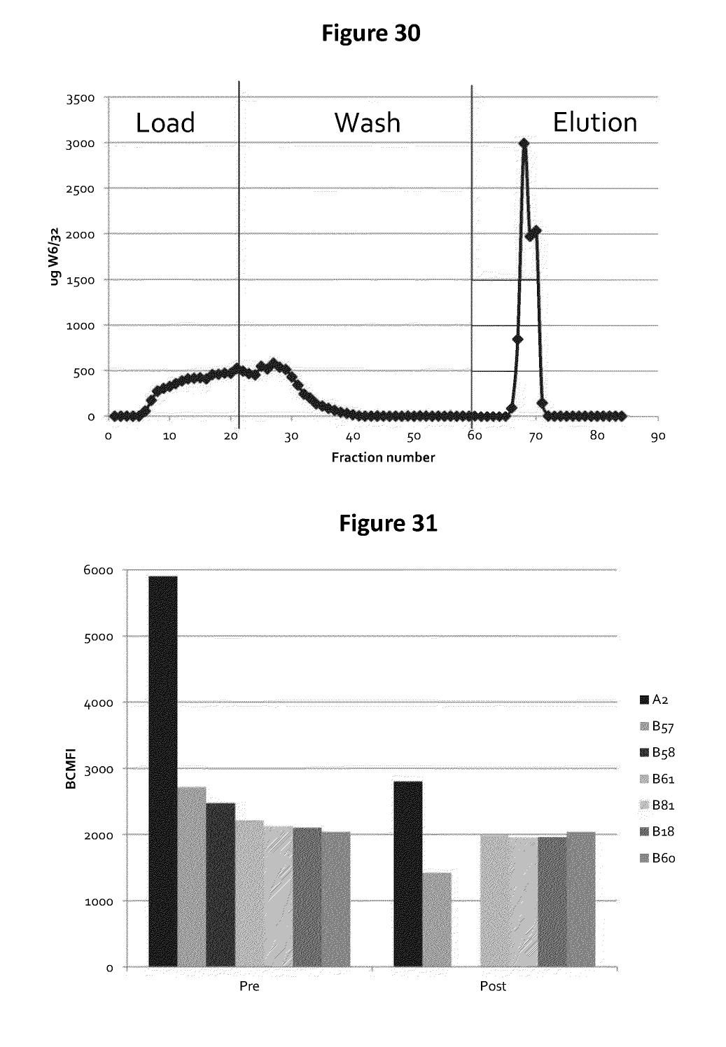

In order to demonstrate that the full scale SHARC was able to deplete anti-HLA antibodies, the ability to deplete monoclonal anti-HLA antibodies from PBS was first investigated. In these experiments, sHLA-A2 was used as the class I molecule, and sHLA-DR11 was used as the class II molecule. The pan-class I antibody W6/32 was used for analysis of class I, while the pan-HLA-DR antibody L243 was used for class II. As shown in FIGS. 30 and 33, both the sHLA-A2 (class I) and sHLA-DR11 (class II) SHARC devices depleted anti-HLA antibodies from PBS, although the sHLA-DR11 SHARC was more effective than the HLA-A2 SHARC.

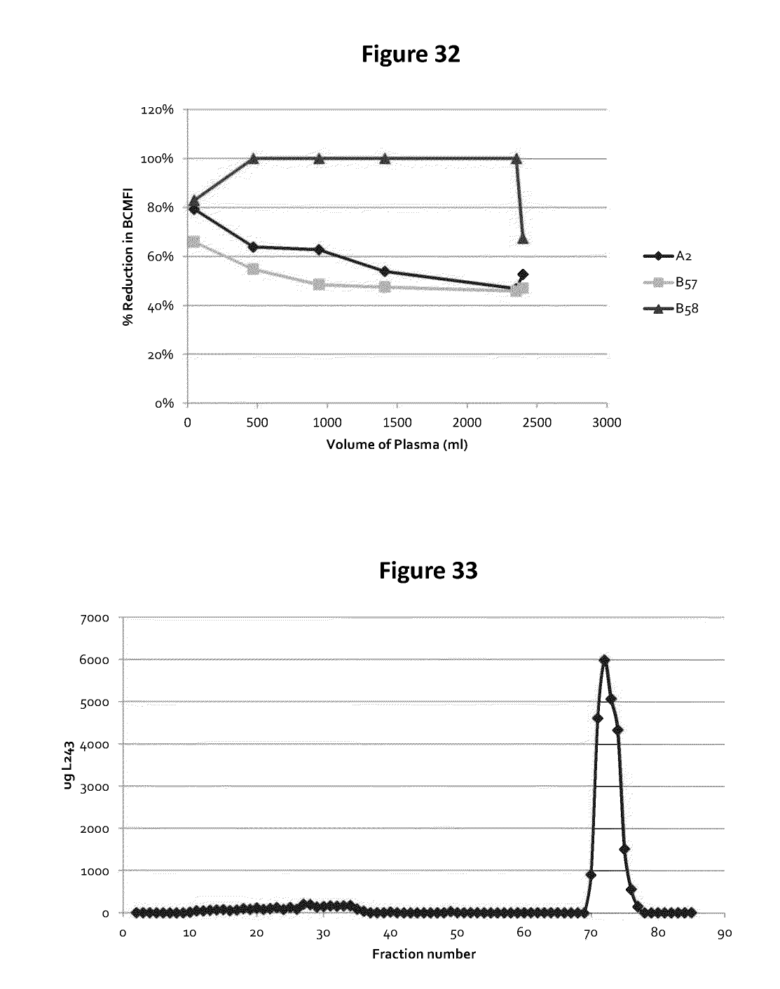

Next, the ability of the class I and II SHARC to deplete antibody from patient plasma containing anti-HLA antibodies was tested. When patient plasma containing anti-HLA-A2 antibodies was passed over the sHLA-A2 SHARC, anti-HLA-A2 antibodies were depleted (FIGS. 31 and 32). In addition to anti-HLA-A2 antibodies, serologically related antibodies (B57, B58) were reduced from the starting plasma. The presence of serologically unrelated anti-HLA antibodies (B61, B81, B18, B60) was unchanged between pre-SHARC and post-SHARC plasma, demonstrating that these antibodies passed through the SHARC without binding thereto (FIG. 31).

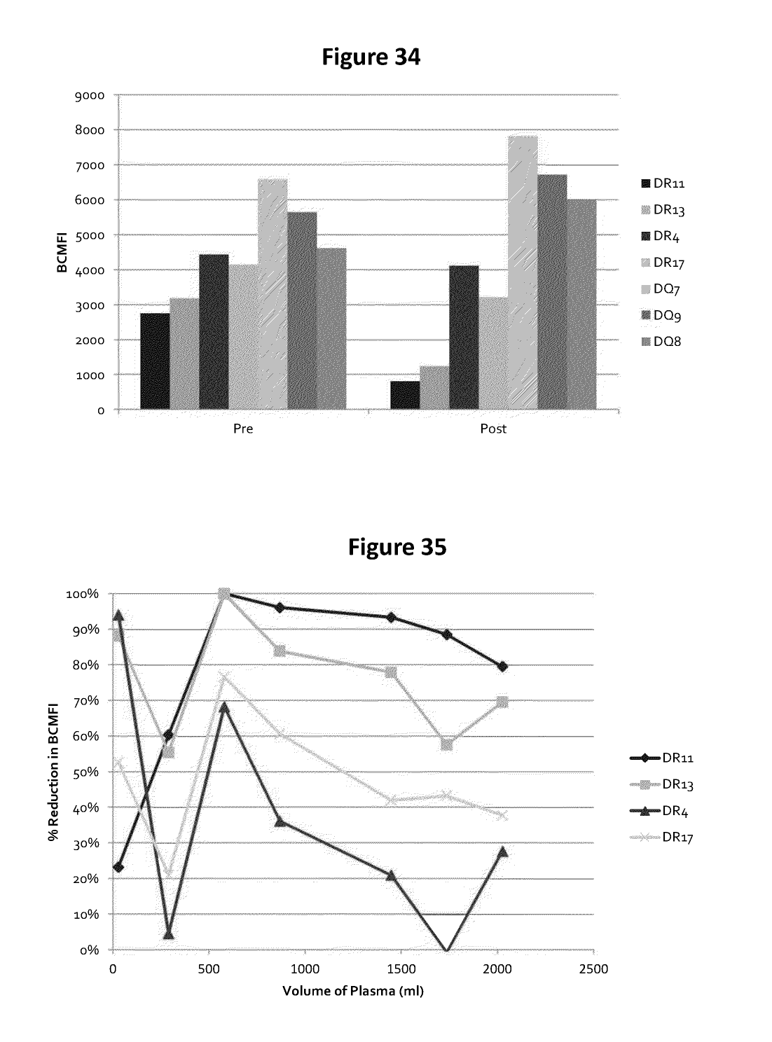

Like the sHLA-A2 SHARC, the sHLA-DR11 SHARC depleted anti-HLA-DR11 antibodies from patient plasma (FIGS. 34 and 35). As shown in FIG. 34, anti-HLA-DR11 antibodies as well as serologically related antibodies (DR13, DR4, DR17) were reduced from the starting plasma. Serologically unrelated anti-HLA antibodies (DQ7, DQ8, DQ9) were unchanged between pre-SHARC and post-SHARC plasma, demonstrating that these antibodies passed through the SHARC without binding thereto. Together these data demonstrate the ability and specificity of both of the class I and II SHARC devices.

### Example 5

### Additional SHARC (sHLA Antibody Removal Column) Analysis

In this Example, several specific HLA-A*0201 columns were generated to demonstrate the feasibility of removing defined anti-HLA antibodies (anti-HLA-mAbs) from a buffered solution. Soluble class I HLA molecules were produced in a native conformation in mammalian cells, purified by affinity chromatography, coupled to a SEPHAROSE® matrix, and loaded into a column enclosure. The HLA on these columns were shown to maintain their structural integrity and function. Multiple passes of the antibody W6/32, which recognizes only intact HLA molecules, resulted in consistent and repeatable binding patterns. During the entire evaluation process, several parameters were identified determining capacity and efficiency. In conclusion, the anti-HLA antibody removal devices have been demonstrated herein to be highly efficient in selectively depleting anti-HLA-mAbs.

### Materials and Methods for Example 5

Recombinant techniques were used to create cell lines which express single HLA class I molecules (as described herein above). Eliminating the cytoplasmic and transmembrane regions of the molecule resulted in a soluble form of HLA (sHLA) which is secreted during production and easily purified by affinity chromatography. Large-scale production of sHLA proteins was performed using the CP-2500 CELL PHARM® system. Hollow fiber bioreactors are designed to produce up to 50 to 100 times more protein than a traditional static culture will yield. Affinity chromatography purification was applied to purify crude sHLA harvests, resulting in samples of >95% purity. All samples produced were individually controlled by a QC system. Mass spectroscopy demonstrated that soluble HLA proteins were purified so that contaminants are essentially undetectable.

After purification of sHLA, fractions of the protein stock were used to couple to NHS-activated SEPHAROSE® 4 Fast Flow and packed into a chromatography column. Elution profiling was conducted using an Äkta Purifier System by applying a specific run protocol consisting of a loading cycle, elution cycle, and equilibration cycle. All parameters were kept consistent throughout the study, assigning a volume of 12 ml of PBS, pH 7.4 to the loading cycle, 8 ml of 0.1 M Glycine pH 11.0 to the elution cycle and 25 ml of PBS, pH 7.4 to equilibrate the system. Depending on the injection amount and volume, different loading loops were used. Data showed that injection conditions are concentration independent (not shown).

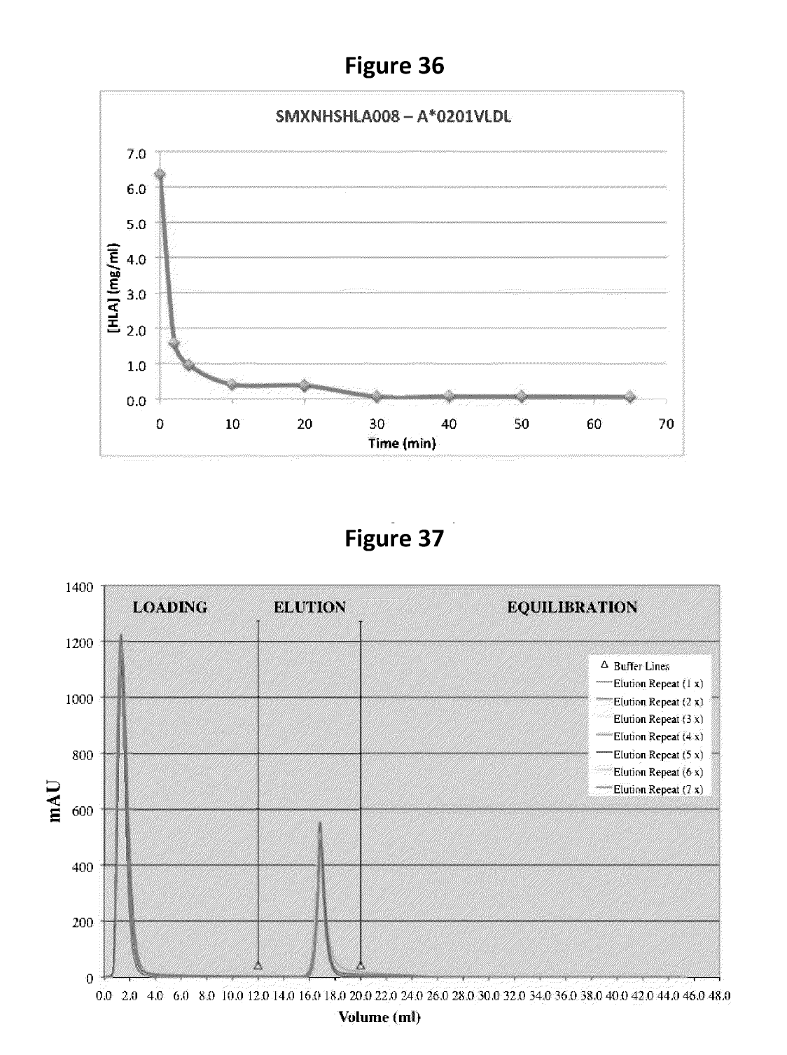

FIG. 36 shows a typical coupling timeline for binding of the sHLA to the SEPHAROSE® 4 Fast Flow matrix. A rapid decline of sHLA is visualized within the first 10 minutes and faded out after 30 minutes, where no additional sHLA is bound to the matrix. For this Example, three columns of 0.5, 1.0 and 2.0 mg per ml matrix were created with coupling efficiencies above 95%.

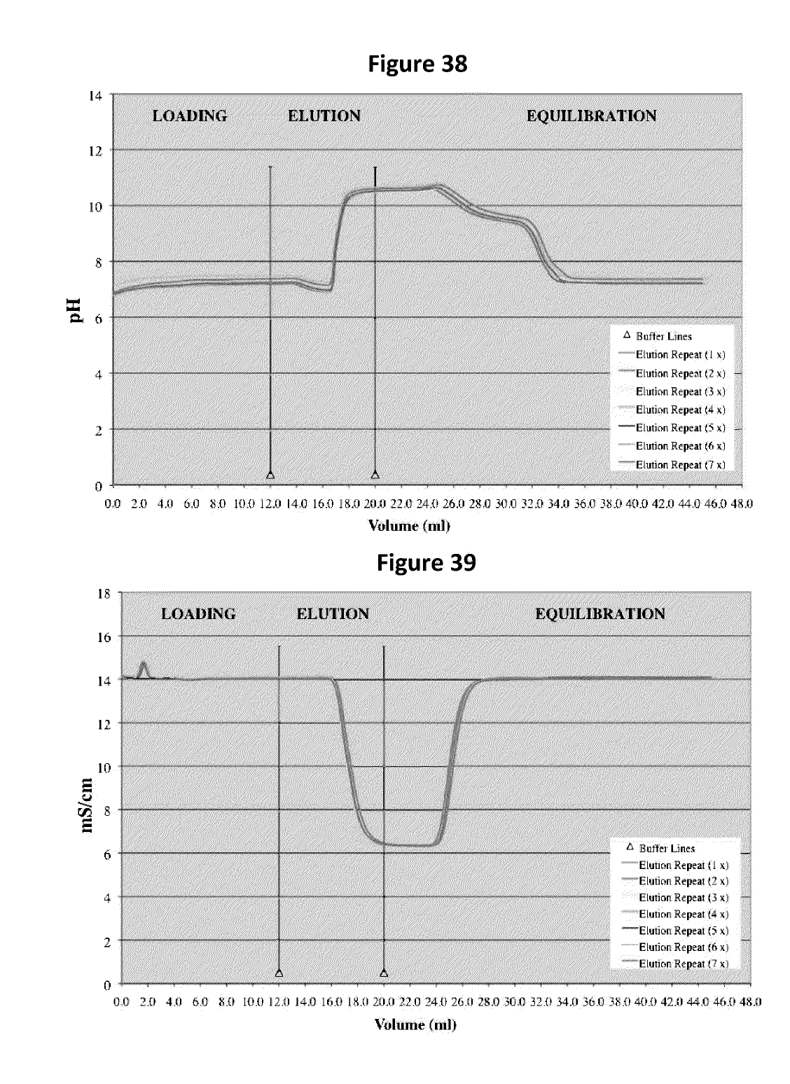

To assure consistency in the measurements, a repeatability study was started to record and superimpose elution profiles. For quality purposes, three parameters were observed: (1) Absorption Units (mAU) to detect proteinacious material (FIG. 37); (2) pH (FIG. 38); and (3) conductivity to follow changes in buffer phases (FIG. 39). The graphics prove great consistency between multiple experiments, validating the suitability of the method.

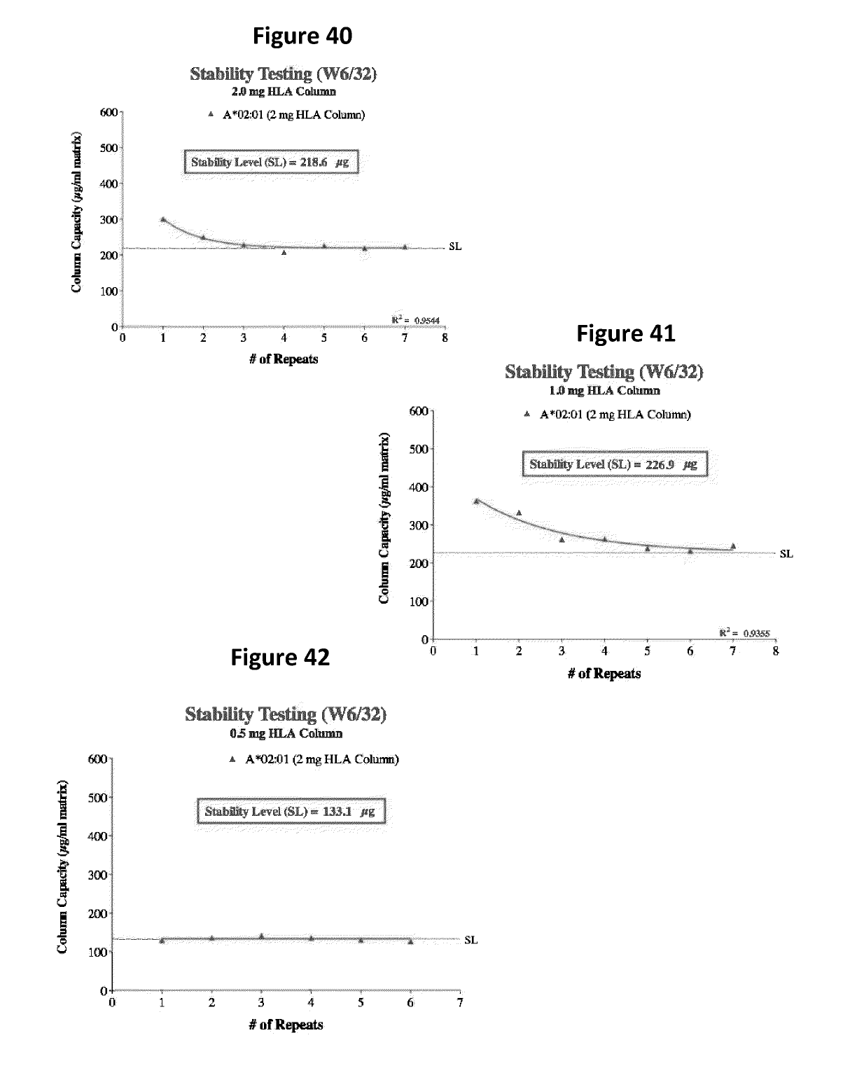

Using the anti-HLA-mAb W6/32, which recognizes only structurally intact HLA molecules, multiple rounds of load-elute-equilibrate cycles were performed to measure the stability of sHLA attached to the solid support (FIGS. 40-42). Overall it was observed that freshly coupled HLA-columns lose HLA molecules within the first 3 rounds of glycine exposure, but then stabilize with no further loss of functionality. This effect is most likely caused by incompletely coupled HLA proteins being trapped within the matrix and knocked loose after a drastic pH change. The effect seems to be more profound in higher coupling ratios. A similar study was performed manually (data not shown), measuring the “shedding” of sHLA after an elution event with the result that no sHLA was detectable after 5 elutions (15 cycles).

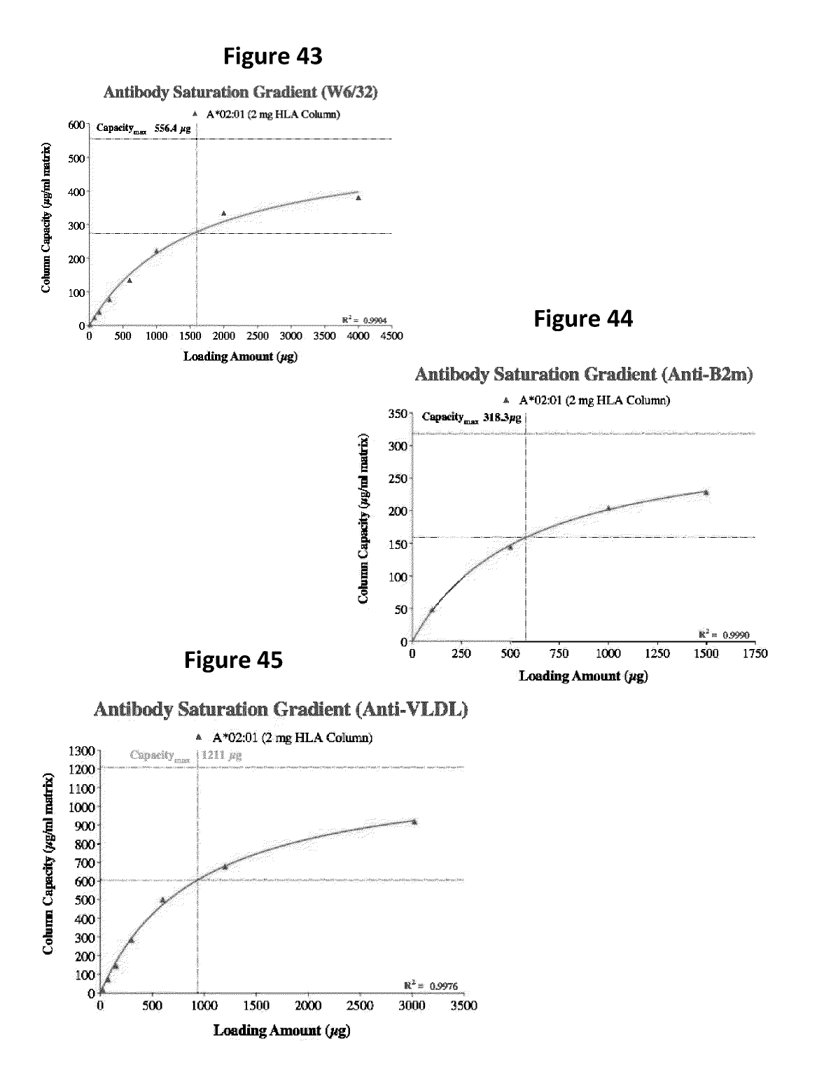

Determination of the column's binding capacity is one of the most important parameters in establishing feasibility of the technology. The more antibody that can be removed, the less sHLA is needed, and smaller/cheaper devices can be created. FIGS. 43-45 show three different anti-HLA mAbs applied to a 2.0 mg column at variable amounts. The column's capacity was shown to not be unlimited, but was able to bind a certain amount of antibodies before saturation occurred. Anti-VLDL (FIG. 45) appeared to be able to bind the largest amount of antibody before the column becomes saturated, while Anti-B2m (FIG. 44) bound the lowest amount of antibody before saturation. These differences were expected, as each antibody has a different affinity towards its target epitope. Depending on the anti-HLA mAb used, capacities ranged from 300-1200 μg of antibody per 1 ml matrix.

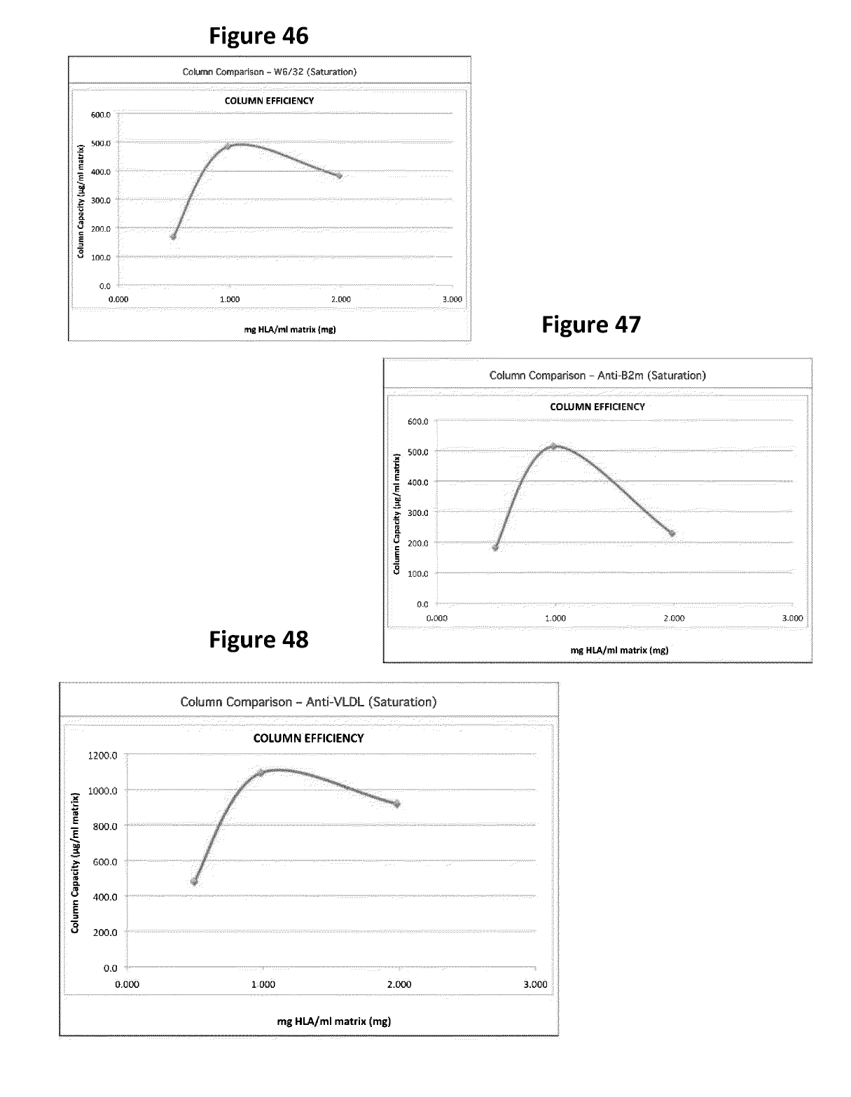

The maximum binding efficiency for the A*02:01 appeared to be at around 1 mg of HLA per 1 ml of matrix. This was confirmed by 3 independent tests using anti-HLA mAbs W6/32 (FIG. 46), anti-B2m (FIG. 47) and anti-VLDL (an antibody directed against an artificial tail introduced into the A*02:01 molecule; FIG. 48). Clear evidence of sterical hindrance was detectable, where the 1 mg column reached much higher binding capacity than its 2 mg counterpart.

This Example demonstrates that soluble HLA class I molecules coupled to an affinity matrix were capable of binding specific anti-HLA Abs. Elution profiles become stable and the column performed without a visual decrease in functionality. All parameters measured were highly acceptable to move forward in creating a large-scale device.

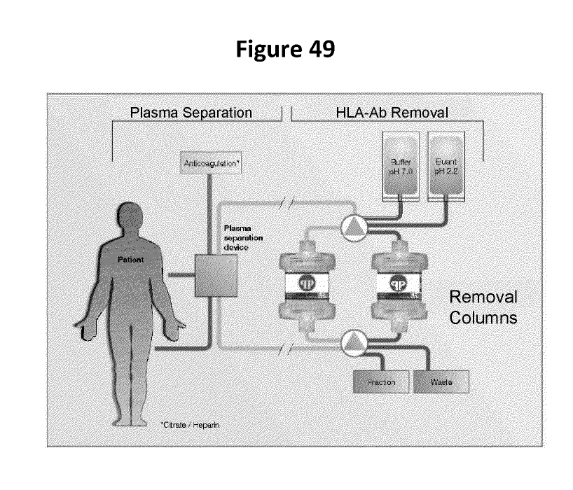

A proposed application scenario using such a system is shown in FIG. 49. The large amount of antibody required to be removed necessitates a two column system where one column is actively filtering plasma while the second is being regenerated.

### Example 6

### Profiling HLA Alloantibodies in Transplant Patient Sera

Antibodies that recognize class I and class II human leukocyte antigens (HLA) represent a contraindication at multiple stages of the organ transplant process. Prior to transplantation, patients who have been sensitized to produce a broad range of HLA-specific antibodies typically wait longer to receive a transplant, and are often limited to desensitization with live donor. Post-transplantation, antibodies that recognize the HLA of the donor organ contribute to hyperacute, acute, and/or chronic rejection of a transplanted organ. These alloantibodies mediate rejection by a number of mechanisms, including but not limited to, activation of the complement cascade, killing via FcγRs following innate immune cell recruitment, inflammation accompanying epithelial cell migration, and epithelial cell apoptosis. While antibodies are recognized as a substantial barrier to allogeneic transplants, antibody responses can differ substantially depending upon the antigen in question, the route of immunization, and the immune status of the responder; substantial heterogeneity can be expected in humoral immune responses to HLA. Indeed, variability among allogeneic immune responses has likely contributed to the observation that not all antibodies that recognize HLA promote organ failure, and a more thorough understanding of anti-HLA antibodies in transplant patients would contribute to understanding those immunoglobulins that are truly a contraindication for transplantation.

Experimentally, the phenotypic and functional evaluation of antibodies directed towards any given HLA molecule remains challenging for several reasons. First, anti-HLA antibody responses can be polyclonal, potentially recognizing multiple epitopes on an allogeneic HLA. Second, sensitized individuals frequently have antibodies reactive with multiple HLA, whereby it is not clear whether one antibody response is serologically cross-reactive with various HLA, or whether individual serologic responses target different HLA. Third, anti-HLA antibodies can be difficult to characterize, as they are intermingled in a complex blend of serum immunoglobulins. Clearly, the isolation of antibodies to a given HLA molecule would enable subsequent studies of anti-HLA antibody concentration, isotype, epitope specificity, and affinity. Such measurements could then be compared to transplant function/survival in order to correlate distinct humoral responses with clinical outcomes. In addition to shedding light on how antibody variables influence clinical outcomes, the isolation of anti-HLA antibodies would help to unravel the impact that antibody isotype, concentration, and affinity have on diagnostic bead-based assays—an area of considerable interest. In particular, clinicians and HLA laboratory technicians share an interest in assigning an antibody titer to their HLA-sensitized patients to determine risk, a calculation that is especially affected by antibody heterogeneity.

This Example examined the isolation and profiling of anti-HLA immunoglobulins to determine if person-to-person differences and similarities in alloreactivity could be observed, thus leading to a better understanding of how such variability influences diagnostic tests and clinical outcomes. In this Example, appreciable quantities of soluble class II HLA molecules were produced in a native conformation and used to construct a novel HLA immunoaffinity column. Patient sera containing a complex mixture of antibodies, including anti-HLA immunoglobulins, were then passed over this column. Antibodies specific for a particular class II HLA were retained on the column; these immunoglobulins were recovered by elution and then profiled for concentration, isotype, cross-reactivity, complement activation, and impact on antibody screening assay outcomes. The resulting phenotypic and functional profiles represent a substantial advance in the understanding of anti-HLA antibody variability, providing new insight as to how immunoglobulin heterogeneity can influence diagnostic tests and transplant outcomes. More robust applications of this HLA antibody isolation and profiling technology are described, including the provision of a new generation of therapeutic antibody removal tools for patients with strong antibody reactivity directed towards allogeneic HLA.

### Methods of Example 6

Patient samples: Patient ‘G’ serum was purchased as HLA-DR11 antiserum with complement fixing activity (Gen-Probe GTI Diagnostics). Patient 1 serum was collected from a DR11 sensitized kidney recipient using informed consent according to a protocol approved by the University of Texas Southwestern Institutional Review Board. Patients 2-12 serum was collected from sensitized patients using informed consent according to a protocol approved by the University of Warwick Institutional Review Board. For patients 13-14, approximately 600 ml of double filtration plasmapheresis retentate from a sensitized patient was recovered after a single session according to a protocol approved by the University of Warwick Institutional Review Board. For Patient 13, 600 ml of retentate was diluted in 1.8 L of PBS; for patient 14, 600 ml of retentate was diluted in 1.8 L HLA antibody negative plasma; and for patient 15, 350 ml of retentate was diluted in 1.8 L HLA antibody negative plasma. HLA antibody negative plasma was obtained by pooling plasma from random blood donors who were confirmed negative by the single antigen bead (SAB) assay.

sHLA-DR11 Protein Production: To produce secreted HLA-DR11 (sHLA) molecules, α-chain cDNAs of HLA-DRA1*01:01 and HLA-DRB1*11:01 were modified by PCR mutagenesis to delete codons encoding the transmembrane and cytoplasmic domains and to add the leucine zipper domains. For DRA*01:01, a 7 amino acid linker (DVGGGGG; SEQ ID NO:7) followed by leucine zipper ACIDp1 was added. For DRB*11:01 the same linker was used, followed by leucine zipper BASEp1 sequence. sHLA-DRA1*01:01 and sHLA-DRB1*11:01 were cloned into the mammalian expression vector pcDNA3.1(−) geneticin and zeocin, respectively (Invitrogen). The HLA class II deficient B-LCL cell line NS1 (ATCC # TIB-18) was transfected by electroporation simultaneously with sHLA-DRB1*11:01 and DRA1*01:01. Drug resistant stable transfectants were tested for production of sHLA class II molecules by sandwich ELISA using L243 (Leinco Technologies) as a capture and class II specific commercial antibody for detection (One Lambda Class II, One Lambda Inc.). The highest producing clone was expanded and seeded into an ACUSYST-MAXIMIZER® hollow fiber bioreactor (Biovest International, Worcester, Mass.). Approximately 75 mg of sHLA-DR11 was purified from the harvest using an L243 immunoafffinity column with an alkaline elution. Purified sHLA-DR11 was quantified using a standard BCA assay.

Mass spectrometry: 10 μg of purified sHLA-DR11 was reduced and denatured with dithiothreitol (Sigma-Aldrich D0632) and incubated at 95° C. for 5 minutes. Sample was then alkylated with iodoacetamide (Thermo Scientific 89671F), for 1 hour at room temperature. Denatured protein was digested with trypsin using a standard two step digestion protocol (Thermo Scientific 90055). Tryptic peptides were reconstituted in 30% acetic acid/70% ultra-pure water, and loaded onto the UltiMate® 3000 HPLC system (Dionex, Sunnyvale, Calif.) with a PepMap100 C18 75 μm×15 cm, 3 μm 100 Å reverse phase column. Peptides were eluted and analyzed on a QTOF Qstar Elite mass spectrometer (ABI MDS Sciex) with Mascot software.

sHLA-DR11 Affinity Columns: Two different size columns were used in this Example; a small 1 ml gravity column and a large 65 ml pump flow column. In both cases, the sHLA-DR11 was coupled to NHS-activated SEPHAROSE® 4 Fast Flow matrix at a ratio of 1 mg of sHLA-DR11 per 1 ml of matrix according to manufacturer's protocol. For the small column, 1 ml of coupled matrix was packed into a 1 cm diameter glass gravity column enclosure. For the large 65 ml column, matrix was packed into an empty GLYCOSORB® column enclosure (Glycorex International AB, Lund, Sweden).

Immunoaffinity Purification of Alloantibodies: On patients 1-12 and patient ‘G’, antibodies were purified from the sera by passing 1 ml of undiluted sera over the 1 ml gravity column. The column was then washed with 7 ml of PBS, pH 7.4, followed by an elution with 4 ml of 0.1 M glycine, pH 11.0. Eluate was instantly neutralized in 1M TRIS, pH 7.0, at a ratio of 1:5 TRIS:Eluate. On patients 13-15, approximately 2.5 L of plasma containing alloantibodies were passed once over the 65 ml column at an average flow rate of 35 ml/min. The column was then washed with 1 L of PBS, pH 7.4, and antibodies were eluted with a total volume of 240 ml of 0.1 M glycine, pH 11.0. As with the small scale columns, eluate was instantly neutralized in 1 M TRIS, pH 7.0, at a ratio of 1:5 TRIS:eluate. After each load/elution cycle, the columns were mock eluted with 0.1 M glycine, pH 11.0, followed by a wash in PBS, pH 7.4.

For the L243 experiment (FIG. 19), 1 ml of L243 at 200 μg/ml was applied to the matrix and allowed to be absorbed by gravity. After sample application, 4 ml of PBS, pH 7.4, was added. During this loading step, 25 fractions were collected manually, each containing ˜200 μl. Finally, the column was eluted by applying 5 ml of 50 mM DEA, pH 11.3. 20 fractions were collected in the elution process and immediately neutralized with 35 μl of 1 M TRIS. For L243, collected fractions were measured by OD₂₈₀ for antibody content.

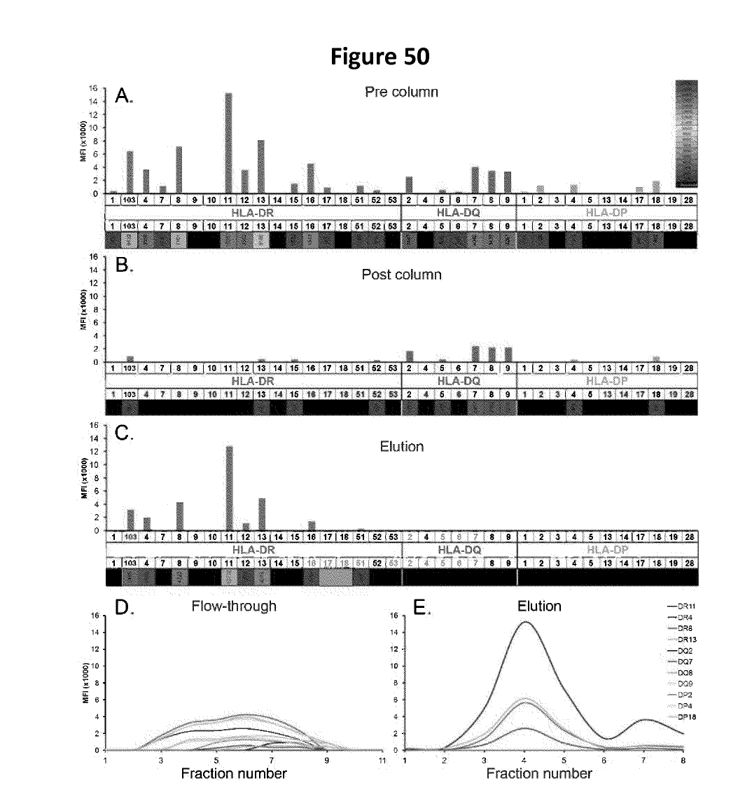

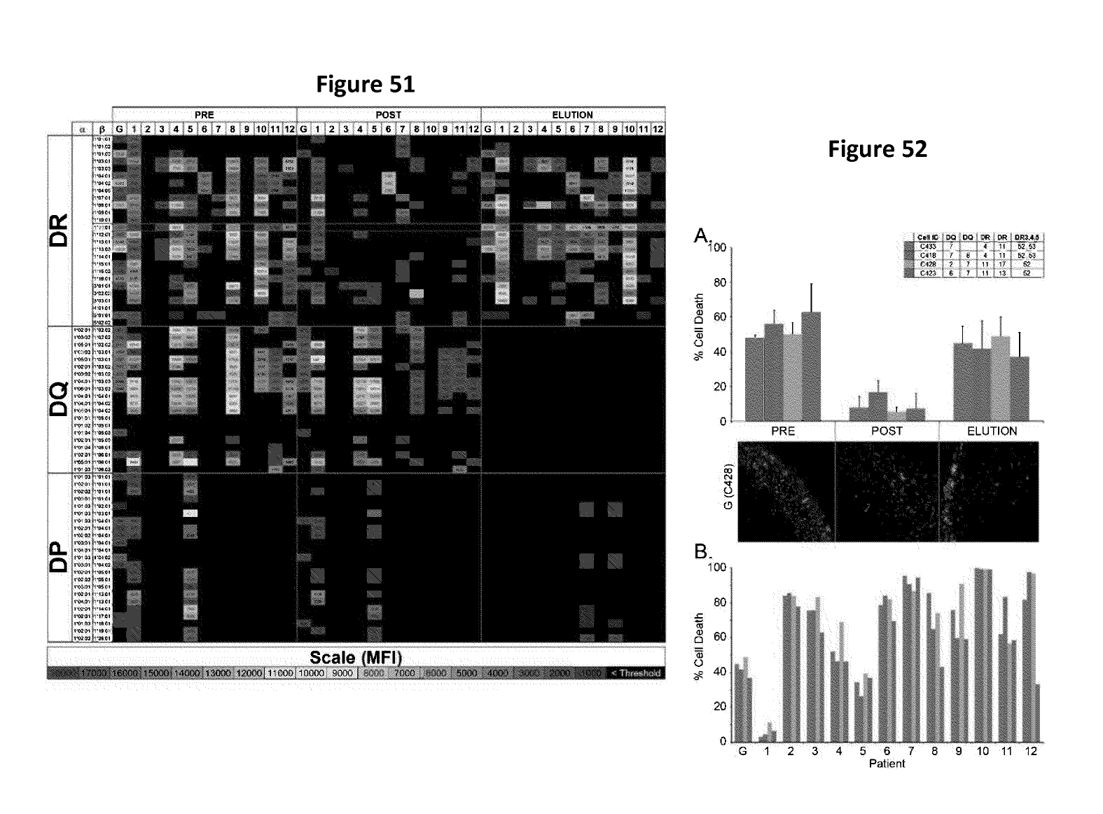

Class II HLA Single Antigen Bead Assays: For the experiments shown in FIGS. 50 and 51, specificities of anti-HLA antibodies in the pre-column serum, flow through, and eluate were determined using LIFECODES® LSA™ Class II HLA single antigen assay (Hologic Gen-Probe Molecular Diagnostics, San Diego, Calif.), according to manufacturer's protocols. Briefly, 40 μl of the bead suspension was incubated with 10 μl of the test sample at room temperature for 30 minutes in the dark on an orbital shaker. Beads were washed and incubated with supplied LSA Conjugate Concentrate (goat anti-human IgG PE (diluted ten-fold)) for 30 minutes in the dark on an orbital shaker.

For every other experiment, a single lot of LABScreen® Class II Single Antigen Beads (Lot#009) were used to determine MFI values (One Lambda). Briefly, 2.5 μl of the bead suspension was incubated with 10 μl of sample and incubated at room temperature for 30 minutes in the dark on an orbital shaker. Using a filter plate, beads were washed with the supplied wash buffer and incubated with 50 μl the detecting antibody (anti-human IgG PE secondary antibody (diluted 100-fold) supplied by One Lambda) at room temperature for 30 minutes in the dark on an orbital shaker. After incubation with the secondary antibody, the beads were washed and analyzed on a LUMINEX® 100 analyzer.

All MFI values for every sample were normalized using the positive control beads according to the following equation: Allomorph MFI*(20000/Positive control bead MFI).

Immunoglobulin lsotyping: For antibody isotyping and quantification, the BIO-PLEX PRO™ immunoglobulin isotyping kit (Bio-Rad Laboratories, Inc., Hercules, Calif.) was used according to manufacturer's protocols. Briefly, 10-fold serial dilutions of the sample were made, and 50 μl of the sample was incubated with 50 μl of the bead suspension for 30 minutes at room temperature. Beads were washed and incubated with a biotynlated secondary antibody at room temperature for 30 minutes. Beads were then washed and incubated with streptavidin PE at room temperature for 30 minutes. Lastly, beads were washed and analyzed on a LUMINEX® 100 analyzer (Luminex Corp., Austin, Tex.). Sample MFI values were translated into Ig concentration using the Ig specific standard curves generated from Ig mixes supplied by the manufacturer. All calculations were made using the BIO-PLEX MANAGER™ software (Bio-Rad Laboratories, Inc., Hercules, Calif.).

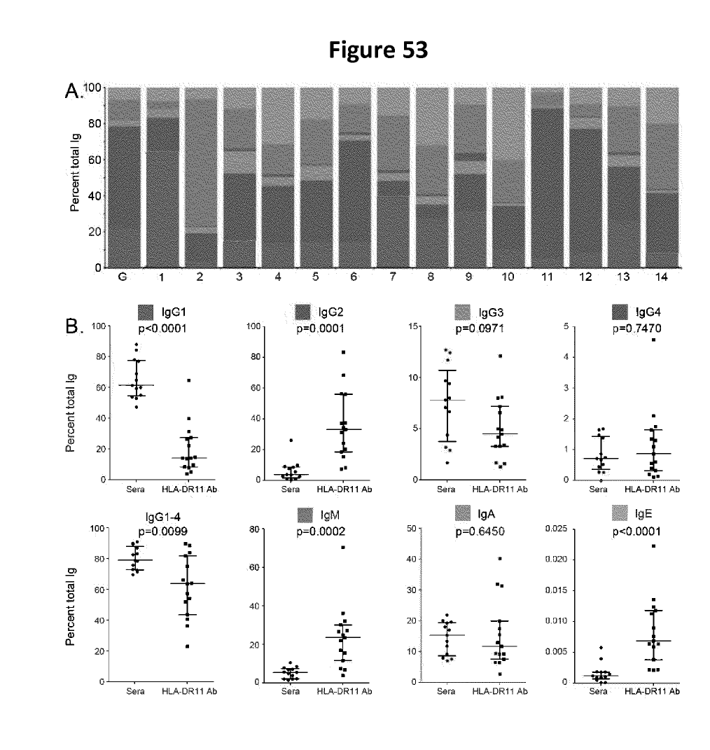

Complement Dependent Cytolysis: Complement dependant cytolysis (CDC) was determined using the Lambda Cell Tray: 30 B cell panel (One Lambda Inc.) Cell lines analyzed were DR11 positive. Cell line class II HLA haplotypes are shown in FIG. 53. Lysis was performed on indicated samples according to manufacturer'S protocols. Rabbit complement was used as a source of complement. After lysis, FluoroQuench™ dye (One Lambda Inc., Canoga Park, Calif.) was used to differentiate live cells from lysed cells. Live cells and lysed cells were then analyzed using a Nikon TE200-E florescent microscope. Whole well images were generated for each well using the 4× objective lens for both the green filter (excitation: 490 nm by 20, emission: 520 nm by 38) and the red filter (excitation: 555 nm bp 28, emission: 617 nm bp 73). Total florescence in both channels was determined using MetaMorph v 7.5.5.0 software, and percent cell death was calculated as: red florescence/(red florescence+green florescence).

Size Exclusion Chromatography: IgM and IgA multimers were separated from monomeric Ig using size exclusion chromatography. Antibodies were either left neat or reduced with 100 mM DTT overnight at 4° C. Human IgM, IgA, IgG was obtained from Sigma-Aldrich as >95% pure. 10 μg (10 μl at 1 mg/ml) of human IgM, IgA, IgG, or purified alloantibodies were injected into a Michrome HPLC and run over a Phenomenex BioSep™ SEC-s4000 SEC column (4.6 mm ID×300 mm length) at a flow rate of 220 μl. Chromatograms were made by measuring the absorbance at 215 nM of the eluting species.

Statistical Analysis: Data variance was determined using a D'Agostino and Pearson omnibus normality test. On parametrically distributed data, mean and standard deviation was used to describe the data. Significant differences in mean values were determined using an unpaired t-test. On non-parametrically distributed data, medians and interquartile range were used to describe the data. Significant differences in median values were determined using a Mann-Whitney test.

### Results of Example 6

Generation of HLA class II Immunoaffinity Column: The isolation of anti-class II HLA antibodies requires a source of plentiful, native class II HLA. In this Example, DNA constructs for a secreted HLA-DRA1*01:01/HLA-DRB1*11:01 alpha/beta heterodimer were prepared by replacing the transmembrane domain of the alpha and beta chains with a 7 amino acid linker followed by an ACIDp1 or BASEp1 leucine zipper domain, respectively (FIG. 18A). This approach was implemented so that (1) the lack of a transmembrane domain would make the class II complex soluble, whereby transfected mammalian cells would continually secrete the desired alpha/beta complex, and (2) the leucine zipper domain would bring and keep the HLA-DRA1*01:01/HLA-DRB1*11:01 heterodimer together in solution. Additionally, a non-human mammalian cell line was used for sHLA-DR11 production to prevent the endogenous non-human class II MHC alpha and beta proteins from pairing with the transfected, soluble, alpha/beta HLA proteins.

To confirm that the secreted class II HLA molecules purified from tissue culture harvests were indeed HLA-DRA1*01:01/HLA-DRB1*11:01 heterodimers, the purified class II protein was digested with trypsin and subjected to liquid chromatography mass spectrometry (LCMS) analysis. In a BLAST analysis, the only protein sequences detected were derived from the transfected sHLA-DR11 alpha and beta chains of the class II complexes produced and isolated here (FIG. 18B). The desired alpha and beta chain of sHLA-DR11 were therefore produced and purified without contamination from other class II MHC subunits.

Pure sHLA-DR11 was covalently coupled to SEPHAROSE® 4 Fast Flow to create an immunoaffinity column. In order to confirm the serologic activity of secreted class II HLA following its coupling to a column, the anti-HLA-DR monoclonal antibody L243 that recognizes intact class II HLA proteins was passed over the HLA affinity column. Fractions of 200 μl were collected during the L243 loading process (flow through), and L243 antibodies that bound to the class II HLA on the column were then eluted from the column intact. A total of 200 μg of L243 was passed over the HLA-DR11 column, 170.6 μg (78%) of which was recovered: 122.9 μg (72%) bound to the class II HLA column and was recovered in the eluate, while 47.8 μg (28%) passed through the column and was recovered in the flow through (FIG. 19A). Furthermore, the captured and eluted L243 antibody maintained its HLA-DR binding activity and specificity in an HLA single antigen bead assay (SAB) (FIG. 19B). These data demonstrate that sHLA-DR11 retains a native conformation when coupled to an affinity column matrix and that a sHLA-DR11 column can be used to recover anti-HLA antibodies that are intact and suitable for use in immunoassays.

Affinity Purification of Alloantibodies from Sensitized Patient Sera: Having demonstrated with monoclonal antibody L243 that sHLA-DR11 complexes were serologically active on an immunoaffinity column, alloantibodies were next passed over the DR11 column. Alloantibodies can be quite complex, so initially a well-defined commercial sera was column purified. For this first run, a commercial sera (GTI Diagnostics) that is cytotoxic to only DR11 expressing cell lines was passed over the column. Prior to passage over the DR11 column, the GTI DR11 serum was found to be cross-reactive with DR, DQ, and DP specificities (FIG. 50A). Following passage through the DR11 column, the majority of the DR reactive antibodies bound to the column, while DP and DQ specificities flowed through the DR11 column (FIGS. 50B and 50D). Antibodies to DR11 that were bound and then eluted from the column did not react to DQ or DP (FIGS. 50C and 50E). The recognition of several HLA-DR by the antibodies purified with the DR11 column demonstrates that amino acids 70DA and 37YV of the class II beta chain represent serologic epitopes for this commercial sera. These data demonstrate that epitope-specific antibodies can be isolated from sensitized sera using HLA immunoaffinity chromatography.

Next, a panel of DR11 sensitized patient sera was passed over the immunoaffinity column (Table 3). For each of twelve patients, antibodies in the pre-column sera, post-column flow through, and antibody eluate were compared using an HLA single antigen bead assay (FIG. 51). In all but one high-titer individual, the HLA column completely depleted DR11 specificities from the sera. From every individual but patient 2, DR11 antibodies, as well as other DR cross-reactive specificities, were recovered in the column eluate. These eluted antibodies reacted with multiple DR in the single antigen bead assay, whereby patterns of DR cross-reactivity were consistent with known DR serologic epitopes. For example, antibodies purified from patients 7 and 9 reacted with both DR and DP due to the shared 57/55DE epitope. Patients 7 and 9 also exhibited specificities in their purified antibodies that were not in the starting sera, an observation explained by the fact that purified HLA antibodies have a reduced background in the SAB assay as compared to raw sera; purified antibody MFIs fall significantly above a greatly reduced background signal. There was no evidence of DQ activity in the recovered/purified antibodies (FIG. 51), and the DR11 serologic epitopes recognized by patient antibodies could be defined on the basis of cross-reactivity with other DR/DP molecules.

Serum Concentration of HLA-DR11 Alloantibodies: The class II HLA column removed most if not all of the DR11 reactivity in the 12 patients tested. When the total antibody recovered from the column was assessed after from running 1 ml of sera over the column, and column loss was adjusted to approximately 25%, the serum concentration of DR11 alloantibodies ranged from a high of 26.1 μg/ml of sera to a low of 0.76 μg/ml with a median concentration of 2.3 μg/ml. The average bulk serum Ig concentration was 48.6 mg/ml in this cohort: between 0.002% and 0.054% of total serum Ig were DR11 reactive alloantibodies.

TABLE 3

| | | | | | | | Previous | | DR11 IgG | DR11 | DR11 |
|---|---|---|---|---|---|---|---|---|---|---|---|
| Sample | Age* | DR1 | DR1 | DR345 | DQ | DQ | Tx | Probable DR Sensitizing Events | FXM | IgM FXM | SAB |
| GTI | Unk | | | | | | Unk | Unknown | N.T. | N.T. | + |
| 1 | 50 | | | | | | 1 | First graft DR4 DR11 mismatch | N.T. | N.T. | + |
| 2 | 61 | 7 | 13 | 52, 53 | 2 | 7 | 0 | No known sentitizing event | + | + | − |
| 3 | 29 | 1 | 7 | 53 | 2 | 5 | 3 | First graft no DR mismatch. Four pregnancies; | − | − | + |
| | | | | | | | | partner type unknown | | | |
| 4 | 42 | 1 | 4 | 53 | 5 | 8 | 1 | First graft DR7 mismatch | − | − | + |
| 5 | 43 | 1 | 4 | 53 | 5 | 8 | 1 | First graft type unknown | + | − | + |
| 6 | 22 | 9 | 17 | 52, 53 | 2 | 9 | 1 | First graft DR4 mismatch | − | − | + |
| 7 | 65 | 4 | 17 | 52, 53 | 2 | 7 | 0 | Two pregnancies; partner DR7 | + | − | − |
| 8 | 34 | 10 | 15 | 51 | | | 1 | First graft type unknown | + | + | + |
| 9 | 43 | 13 | 13 | 52 | | | 0 | Two pregnancies; partner type unknown | + | − | − |
| 10 | 47 | 1 | | | 5 | | 0 | No known sentitizing event | − | − | + |
| 11 | 61 | 1 | 17 | | 2 | 5 | 1 | First graft DR7 mismatch | + | − | + |
| 12 | 48 | 1 | 7 | | 2 | 5 | 1 | First graft DR17 mismatch | + | − | + |
| 13 | 75 | 9 | 17 | 52, 53 | 2 | 9 | 0 | Blood transfusion; type unknown | + | − | + |
| 14 | 62 | 4 | 17 | 52, 53 | 2 | 7 | 1 | First graft DR11 mismatch | + | − | + |

*at time of blood draw

Istotype Profile of HLA-DR11 Alloantibodies: Column-isolated HLA-DR11 reactive antibodies were next profiled for their immunoglobulin isotype. All Ig istotypes were detected in all patient sera, including IgG, IgM, IgA, and IgE (FIG. 52A). The ratio of these antibodies differed from patient to patient—patient 1 had a substantial proportion of IgG1, while in patient 2, greater than 70% of the DR11 reactive antibodies were IgM. Nonetheless, when compared to bulk Ig, this cohort showed a consistent pattern, whereby the IgG1 isotype was significantly underrepresented and IgG2 was overrepresented. DR11 IgG3 and 4 levels were unchanged as compared to total serum IgG (FIG. 52B). When added together, the IgG istotypes were under represented, accounting for 64% of the total DR11 Ig compared to serum IgG isotypes that represented 79% of the total Ig. The IgM isotype compensated for this drop in IgG levels, as IgM accounted for 31% of total DR11 Ig. IgA represented 12% of the DR11 reactive antibodies, equivalent to the IgA seen in bulk serum Ig. While IgE was less than a hundredth of a percent of the total Ig, it was significantly higher than the proportion found in sera. These data demonstrate that anti HLA-DR11 alloantibodies contain measurable amounts of every isotype, are enriched in IgG2, IgM, and IgE, and that IgG1 is underrepresented.

Purified HLA-DR11 Antibodies Fix Complement: Purified DR11 alloantibodies were next tested for their ability to fix complement. Four different DR11 expressing B-cell lines served as target cells. The relatively simple commercial serum ‘sample G’ had only DR11 specific complement fixation activity (FIG. 53A). After column absorption of DR11 antibodies, complement-fixing activity was recovered in the antibodies eluted from the column, while virtually no cytolysis remained in the sample flow through: Column purified DR11 alloantibodies retain complement-fixing activity. Next, alloantibodies were column purified from the 12 patients and tested for complement-fixing activity (FIG. 53B). Ten of the twelve patient sera exhibited cell lysis of greater than 40%, including antibodies from patient 2 that were predominantly IgM. Patient 5 showed relatively little cytolytic activity, even though this patient's DR11 isotype profile resembled the rest of the group, and patient 1 had no significant CDC activity despite a prevalence of IgG1 alloantibodies—an isotype known to fix complement. Thus, the DR11 isotype profile reported here consistently fixed complement, yet exceptions in two patients demonstrate that additional factors contribute to CDC activity.

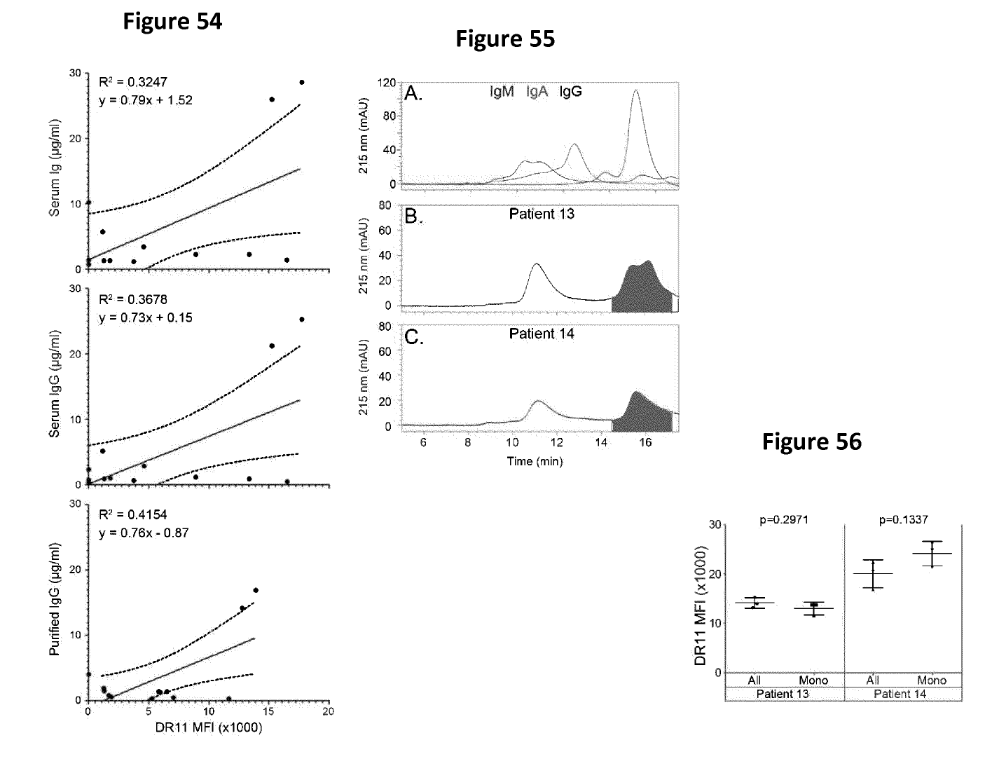

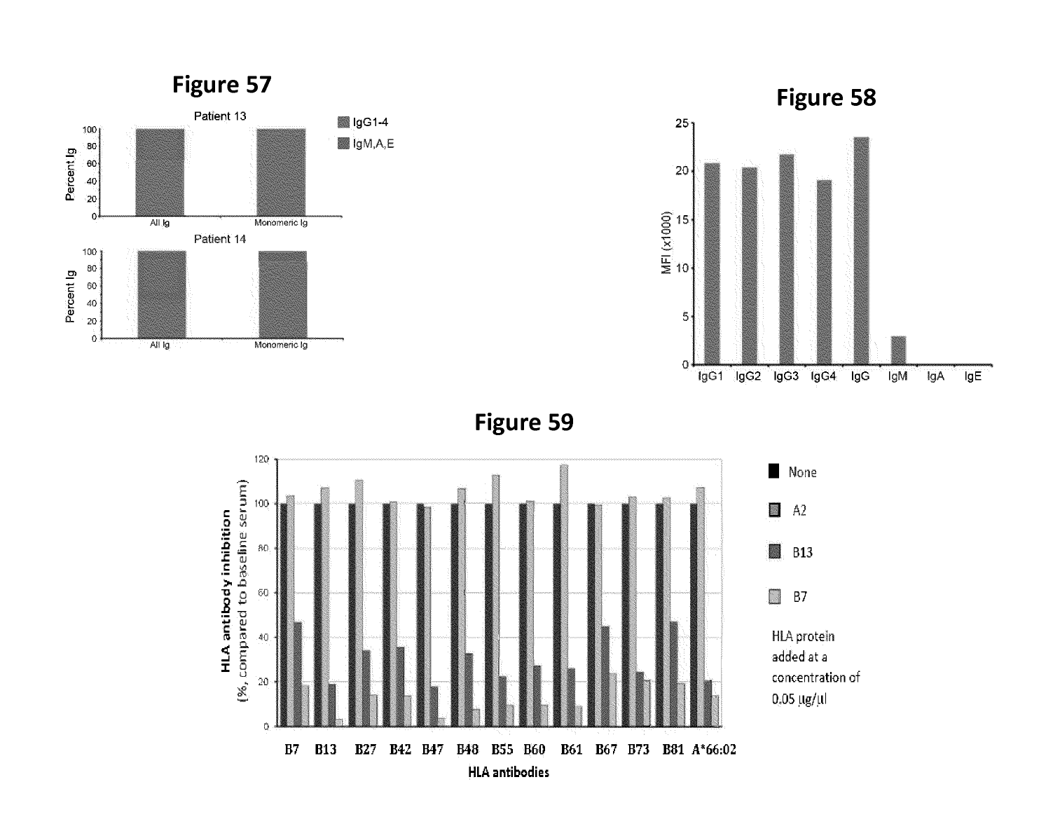

MFI & Antibody Concentration: HLA column purification enabled the determination of alloantibody concentration in DR11 specific patient sera. Hence, it was possible to assess how well MFI values correlated with serum antibody concentration. As stated previously, DR11 reactive antibody concentrations for the 13 patients tested here ranged from 26.1 μg/ml to 0.76 μg/ml. Next, MFI values obtained with the pre-column sera were plotted against serum antibody concentrations. As shown in FIG. 54A, highly variable but significant (R²=0.3247) linear correlation between MFI values and serum antibody concentration was seen. When the data is plotted in terms of the serum IgG concentration (having subtracted IgA, M, & E), a similar correlation is seen, but the variability is slightly reduced (R²=0.3678) (FIG. 54B). With the purified antibodies, the plotted MFI versus IgG variability was even further reduced (R²=0.4154) (FIG. 54C). However, in all of these examples, many patient antibodies fell outside of the 95% confidence bands, including three patients that had relatively low antibody concentrations with consistently high MFI values. Thus, while there is a significant linear correlation between serum antibody concentrations and MFI value, a high degree of variation makes it difficult to determine serum antibody concentrations with MFI values.

The Influence of IgM and IgA Multimers on MFI Values: As observed in the CDC activity assay, multiple factors are positioned to influence the behavior of antibodies in a diagnostic test, including but not limited to, differences in isotype mixtures, antibody affinity, and the breath of epitope specificities recognized. When testing HLA alloantibodies, the removal of IgM multimers by either DTT reduction or size exclusion has been reported to provide more meaningful determinations of IgG concentration. Here, it was hypothesized that MFI values would more accurately reflect patient-to-patient IgG concentrations with IgM and IgA multimers removed. Milligrams of DR11 reactive alloantibody were purified from patients 13 and 14 (the only patients with ≧500 ml of available sample), their IgM and IgA multimers were separated from IgG monomers by size-exclusion chromatography (FIG. 55), and the purified monomeric DR11 reactive antibodies were confirmed as >88% IgG (FIG. 58). These two IgG preparations were adjusted to 20 μg/ml and tested on a SAB assay. Patient 13 had an average MFI value of 10,660, and patient 14 had an MFI of 15,075; a significant (p=0.0285) difference of 4,415 MFI remained between these equilibrated samples. Moreover, DR11 MFI values did not significantly change, even though considerable concentrations of multimeric IgM and IgA were removed. Thus, multimeric IgM and IgA alloantibody had little to no effect on MFI values within a patient, nor did multimer removal make MFI values comparable between patients 13 and 14.

### Discussion of Example 6

Donor specific anti-HLA antibodies represent a pre-transplant contraindication as well as a post-transplant risk for graft loss. While it is clear that antibodies to HLA mediate graft failure and loss, studies suggest that not all anti-HLA antibodies are detrimental to allografts. Substantial heterogeneity likely exists between antibody responses, and there is a great interest in discerning pathogenic anti-HLA antibodies from those that are not a threat to transplanted organs. To date, few experimental tools have been able to provide a detailed profile of the antibodies to HLA such that the antibodies that warrant clinical intervention remain ambiguous with those responses that do not impact clinical outcomes. Here, an HLA-DR11 immunoaffinity column was developed to purify HLA alloantibodies, and, once purified, these antibodies were profiled. This ability to isolate anti-HLA antibodies is positioned to augment both clinical and basic scientific endeavors by unraveling the complex nature of humoral responses to HLA.

Through the recognition of epitopes or eplets that are shared by multiple HLA allomorphs, alloantibodies to HLA are able to cross-react with several different HLA antigens. Here, the purified DR11 alloantibodies were cross-reactive with numerous DR and some DP molecules. In many cases, the broad reactivity of these purified alloantibodies made it difficult to define the multiple epitopes recognized without sequential absorption and/or blocking experiments. Nonetheless, several class II HLA serologic epitopes were readily apparent when the DR11 column purified antibodies were tested. For example, when sera from certain patients were tested with single antigen beads, cross-reactivity between DR11 and multiple DP (HLA-DP2, 3, 04:02, 6, 9, 10, 14, 16, 17, 18, 28) antigens occurred due to a shared 57DE epitope. Epitope 57DE is defined by an Asp at position 57 and a Glu at position 58 on the beta chain of DR11, with the exact same amino acids found at positions 55 and 56 on the DP beta chain. Another commonly observed class II HLA epitope is 10YST, defined by residues Y10, S11, T12, and S13 that are conserved on DR11, DR3, DR13, and DR14. Here, all seven patients who were not DR3, DR13, or DR14 responded to 10YST, while all DR3 (5/15), DR13(2/15), or DR14(0/15) individuals did not react to the 10YST epitope.

Several studies have examined HLA alloantibody isotypes and found that isotypes IgM and IgA are the major components of an allogeneic response. Minority isotypes are, however, difficult to detect, often remaining hidden within complex sera. The microgram quantities of DR11 reactive antibodies purified here helped to elucidate HLA reactive isotypes representing as little as 0.001% of the total Ig, and in each patient tested, detectable amounts of every istotype, including low abundance IgE, were identified. Because the patients in this Example were sensitized via a variety of antigenic exposures, no single alloantibody isotype was under or over represented in every patient when compared with bulk serum antibodies. Nonetheless, the IgG1 isotype, which has high affinity for Fc receptors (FcR), was underrepresented throughout the patient panel, suggesting that IgG-mediated ADCC plays a minor role in the class II alloresponse. Likewise, IgG subtypes 1 and 3 have the highest affinity for C1q, so one might initially predict that a lack of these class II specific alloantibodies results in a low CDC activity as well. However, the relatively large proportion of IgM found in most patients, an isotype that exhibits the highest affinity for C1q due to its pentameric structure, may functionally compensate for any dearth of IgG-mediated Clq interaction. Indeed, when tested for CDC activity, the purified alloantibodies lysed>50% of the DR11 expressing cells in most patients. A high proportion of IgM class II specific antibodies therefore provide ample CDC activity when IgG1 and IgG3 proportions are low.

Solid phase single antigen bead assays are widely used to screen for allomorph specific HLA antibodies in patient sera. Such assays are very effective at determining the presence of reactivity to a given allotype, but it remains difficult to determine antibody concentration or functional relevance using gradations in MFI. Several explanations are offered to explain a lack of correlation between MFI and antibody concentration, and here it was examined how changes to the surrounding antibody milieu impact the correlation of MFI and IgG concentration. It was initially postulated that a lack of consistency between MFI and IgG measurement was largely due to the interference of IgM and IgA multimeric alloantibodies mixed with the IgG antibodies. Indeed, many groups hypothesize that patient-to-patient variability in IgM and IgA antibody concentration contributes to MFI variability. However, following the removal of multimeric antibodies by both chemical reduction and physical separation, MFI values remained largely independent of IgG levels in the patients tested. These data suggest that antibody multimers have only a modest influence on MFI, and those variables such as antibody affinity and epitope specificity must also influence MFI indications of IgG concentration.

The development of an HLA antibody absorption device to deplete humoral immune responses to specific HLA antigens while leaving adaptive immunity otherwise intact is the long-term objective for the HLA immunoaffinity matrix described here. Today, antibody reduction therapies such as plasma exchange deplete bulk antibodies to facilitate transplants for recipients who are otherwise serologically incompatible. The goal of this Example was to add the element of HLA specificity to these existing reduction therapies, removing only the deleterious anti-HLA antibodies in transplant scenarios. Antigen-specific antibody depletion columns are currently used to remove antibodies specific for blood group A and B antigens, and one could envision the removal of antibodies to HLA with a like technology. For example, in this report it was shown that liters of patient plasma can be processed over columns equivalent in proportion to the GLYCOSORB® (Glycorex Transplantation AB, Lund, Sweden) and IMMUNOSORBA™ (Excorim AB Corp., Lund, Sweden) columns used for blood group desensitization. More than 20 mg of DR11 reactive antibody were HLA column-isolated in each of these three patients, significantly reducing MFI values in the plasma flow through (data not shown). The HLA absorption device matrix tested here demonstrates that complement fixing antibodies to HLA can be removed and recovered from patient samples.

In summary, an approach for producing milligram quantities of native class II HLA proteins in mammalian cells has been developed, and these proteins can be coupled to a column support for use in HLA antibody purification. The recovered DR11 reactive antibodies were functionally intact and highly cross-reactive with DR and DP allomorphs. Given purified alloantibodies, it was demonstrated that detectable amounts of every antibody isotype were present in each patient and that the IgG2, IgM, and IgE isotypes tended to be enriched. Although IgG1 and IgG3 levels were not elevated, these HLA alloantibody mixtures remained active in complement fixation assays. Testing of the purified alloantibodies with HLA SAB confirmed the lack or association between MFI values and antibody concentration, an inconsistency not remedied by the removal of multimeric antibody isotypes. Future column studies with other class II and class I allormorphs will be needed to better elucidate the character, reactivity, and quantity of alloantibodies and to better define those antibodies that promote transplant rejection.

### Example 7

### Selective Depletion of HLA Specific Antibodies from Sera Using SEPHAROSE®Columns Containing Immobilized HLA Proteins

The Department of Health has provided targets to increase the rate of transplantation in the UK, but because the number of heart beating deceased donor organs continues to decline, a rise in the rate of live donor renal transplantation is one effective way to meet this demand. There are obstacles to transplantation, and ABO incompatibility has always been of one the major barriers. Similarly, preformed donor human leukocyte antigen (HLA) specific antibodies often either prohibit or complicate transplantation. To put this into context, around 25% of the 6,000 individuals awaiting kidney transplantation in the UK have detectable anti-HLA antibodies. In the UK, around 300 transplants a year are prevented due to HLA antibodies. A range of antibody types have been reported to impact on the success of renal transplantation, but the two main types are blood group (ABO) and HLA specific antibodies.

The immediate aim of antibody incompatible transplantation (AIT) protocols is to avoid hyperacute rejection, usually with the aid of sophisticated laboratory protocols to enable the rapid quantification of donor specific antibody (DSA) levels. The challenge of successfully treating and preventing both acute and chronic rejection remain in AIT.

The identification of donor HLA specific antibody as a causal factor for hyperacute rejection was first made in 1967, and the first efforts to remove DSA and thus treat antibody mediated rejection was made in the mid-1970s using plasma exchange. The principle of plasma exchange is simple. Blood from the transplant recipient is passed through either a filter or a centrifuge in order to isolate the plasma fraction of whole blood. The plasma containing the harmful DSA is then discarded and replaced with donated plasma to effectively replace albumin and neccessary clotting factors. A major disadvantage of this procedure is that it is relatively poorly tolerated by the patient, and, depending on the size of the individual, only around 3-4 liters of plasma can be treated in a single session.

Double filtration plasmapheresis (DFPP) is similar to standard plasma exchange but has the advantage of being slightly better tolerated, with patient tolerance typically around 8 liters per treatment, which is double that tolerated by standard plasma exchange. In DFPP, plasma is removed in the same way as for plasma exchange, but the plasma is then passed through a second filter which is able to trap larger molecules. This allows components such as albumin, some clotting factors, and a range of other lower molecular weight proteins to pass back into the patient.

Human immunoglobulins can also be depleted by protein A immunoadsorption. Plasma is once again removed as for plasma exchange. The plasma is then passed through a column which contains immobilized protein A. Protein A binds human immunoglobulins and is highly selective. In this manner, plasma is returned back to the patient with only the immunoglobulins removed. This treatment is extremely well tolerated, with the treatment of up to 40 liters of plasma in a single session being possible. However, protein A immunoadsorption is very expensive, and not all antibodies are removed efficiently.

All of the aforementioned antibody removal strategies have the disadvantage of being non-selective, i.e., a depletion of all immunoglobulins is experienced. In the setting of ABO antibody incompatible transplantation, an alternative is available. ABO specific adsorption columns (Glycorex, Lund, Sweden) are commercially available that allow anti-ABO antibodies to be removed exclusively. These columns are extremely well tolerated by the patient, with up to 10 liters of plasma per session routinely being treated. Although expensive, these columns are very attractive to physicians involved in blood group incompatible transplantation.

The challenge of the presently disclosed and claimed inventive concept(s) therefore was to design a strategy to selectively deplete HLA specific antibody, thus leaving humoral immunity completely intact. Previously the construction of HLA specific depletion columns has been prevented due to both the lack of sufficient quantities of soluble HLA protein and production of a wide enough spectrum of HLA specificities. Large scale production of these proteins is now available, with milligram quantities of a wide range of HLA proteins expressed in mammalian cell lines.

HLA proteins are the most genetically variable of all human proteins, giving rise to multiple antigens. For HLA class I (A, B, and Cw loci), there are nearly 2,000 distinct protein forms. But in serological terms, these are derived from specific combinations of up to about ten variant epitopes from a total pool of only 103 epitopes. There is therefore considerable cross-reactivity between different HLA types due to shared epitopes. This cross-reactivity can be exploited by selecting a panel of HLA molecules which collectively represent the widest range of known epitopes. It was estimated that the universe of HLA Class I epitopes can be represented in only 33 selected different HLA types.

This Example explores the scientific feasibility of this approach with the ultimate aim of developing a clinically usable column. The inventors have a serum and plasma archive from almost 100 antibody-incompatible renal transplant patients, and resources for high-throughput screening of anti-HLA antibody profiles via single antigen bead assay are fully established. This Example describes initial soluble and mini-column studies which show the feasibilty of epitope specific HLA class I antibody removal.

### Materials and Methods for Example 7

Patients: Serum samples were taken from the inventors' archive of almost 100 HLA AIT patients. The HLA specific antibody profiles of these patients have been elucidated to the highest available resolution by single antigen bead assay.

Soluble Class I HLA Protein Production: Soluble class I HLA was produced as described herein previously.

Class I Single Antigen Bead Assay: HLA class I specific antibodies were analyzed using a recombinant single antigen microbead assay manufactured by One Lambda Inc. (Canoga Park, Calif.) and analyzed on the LUMINEX® xMAP® 200 platform (Luminex Corporation, Austin, Tex.). Antibody binding was measured as raw fluorescence to avoid differences in background binding seen with different sera which disproportionately influences relative fluorescence, a particular problem associated with plasma exchange. All assays were performed using serum/bead ratios in accordance with the manufacturer's instructions. Briefly, 2.5 μl single antigen microbeads were incubated with 10 μl patient serum at room temperature for 30 minutes. Wells were then washed four times with PBS based wash buffer and incubated for a further 30 minutes with phycoerythrin (PE) conjugated goat anti-human IgG. Samples were then washed a further four times and analyzed using the LUMINEX® analyzer (Luminex Corp., Austin, Tex.). Raw median fluorescent intensity (MFI) values were used to determine anti-HLA antibody specificity.

Soluble Phase Inhibition: Patient sera was incubated at room temperature for 30 minutes with soluble HLA protein to give a final protein concentration of 0.05 μg/μl. This concentration was determined by initial dose titration analysis with the aim of reducing HLA specific antibody level by at least 75% (data not shown). Phosphate buffered saline (PBS) solution was added to the same patient samples to act as negative control and to balance the dilution effect of protein addition. Samples were then tested by single antigen bead assay.

Mini-column Coupling Protocol: To prepare a 200 μg HLA protein column, 200 mg freeze-dried CNBr-activated SEPHAROSE® 4 Fast Flow matrix (GE Healthcare, N.J., USA) was swollen and activated using 2 ml 1 mM HCl, pH 3.0, and chilled on ice for 30 minutes. The swollen matrix was then centrifuged at 2000 g for 10 minutes, and supernatant was discarded. The matrix was then resuspended in 2 ml suspension buffer (50 mM HEPES, pH 7.8, 100 mM NaCl), then re-centrifuged at 2000 g for 10 minutes. The matrix was then resuspended in 500 μl suspension buffer to give a final matrix concentration of approximately 2 mg/ml.

Starting concentrations of HLA protein were determined using OD₂₈₀ absorbance measurement. Two hundred milligrams of HLA protein was added to 100 μl matrix and incubated for 2 hours at 4° C., followed by centrifuging at 2000 g for 5 minutes and measuring OD₂₈₀ absorbance of supernatant. A coupling efficiency greater than 80% was the aim, and the protein/matrix incubation was repeated until the desired coupling efficiency was obtained. 1 ml 1 M ethanolamine was added to deactivate any non-reacted matrix residues, followed by incubation at 4° C. overnight. The matrix was then centrifuged at 2000 g for 5 minutes, and the ethanolamine was carefully decanted off. The matrix was resuspended in 1 ml PBS containing 0.05% sodium azide (NaN₃), pH 7.4. The protein coupled matrix was then packed into a 2 ml affinity chromatography column. A negative control column was prepared in parallel using bovine serum albumin (BSA) as an alternative to HLA protein.

Antibody Removal using HLA Protein Columns: Patient serum was tested by class I single antigen bead assay prior to mini-column absorption. One ml of patient serum was then applied to the HLA protein column and allowed to run through by gravity; a further 1 ml sample was applied to the negative control BSA column. The post-column serum fractions were then re-tested with class I single antigen beads.

To analyze the characteristics of antibody eluted from the mini-columns, 5 ml 100 mM glycine, pH 10, was added to the column, and the eluate was immediately neutralized in 1 M Tris-HCl, pH 8.0. Eluted fractions were dialyzed into PBS, pH 7.4, and analyzed using the single antigen bead assay.

### Results of Example 7

Soluble Phase Inhibition: Patient 065 from the University Hospital Coventry and Warwickshire (UHCW) HLA incompatible transplant (AIT) program was used for initial soluble phase inhibition studies. This patient displayed a single anti-HLA class I specific antibody which recognised the 163E+166E epitope expressed by the following HLA protein specificities: HLA-B7, B13, B27, B42, B47, B48, B55, B60, B61, B67, B73, B81, and A*66:02. This alloantibody was stimulated by a HLA-B7 mismatch from an earlier failed renal transplant. Patient sera was absorbed with soluble HLA-B7 protein at a concentration of 0.05 μg/μl for 30 minutes and analyzed with a single antigen bead assay. Highly specific antibody reduction was seen for all HLA specificities carrying the 163E+166E epitope (FIG. 59), displayed as percentage reduction in antibody reactivity when compared with a comparatively diluted serum sample. The analysis was then repeated using a second HLA protein, HLA-B13, which expresses the correct epitope (FIG. 59). Again, highly effective epitope specific inhibition of the anti-HLA antibody response was observed. The absorption was carried out once more, this time using a HLA protein, HLA-A2, which is negative for the 163E+166E epitope (FIG. 59). No reduction of the 163E+166E specific response was observed using HLA-A2 absorption.

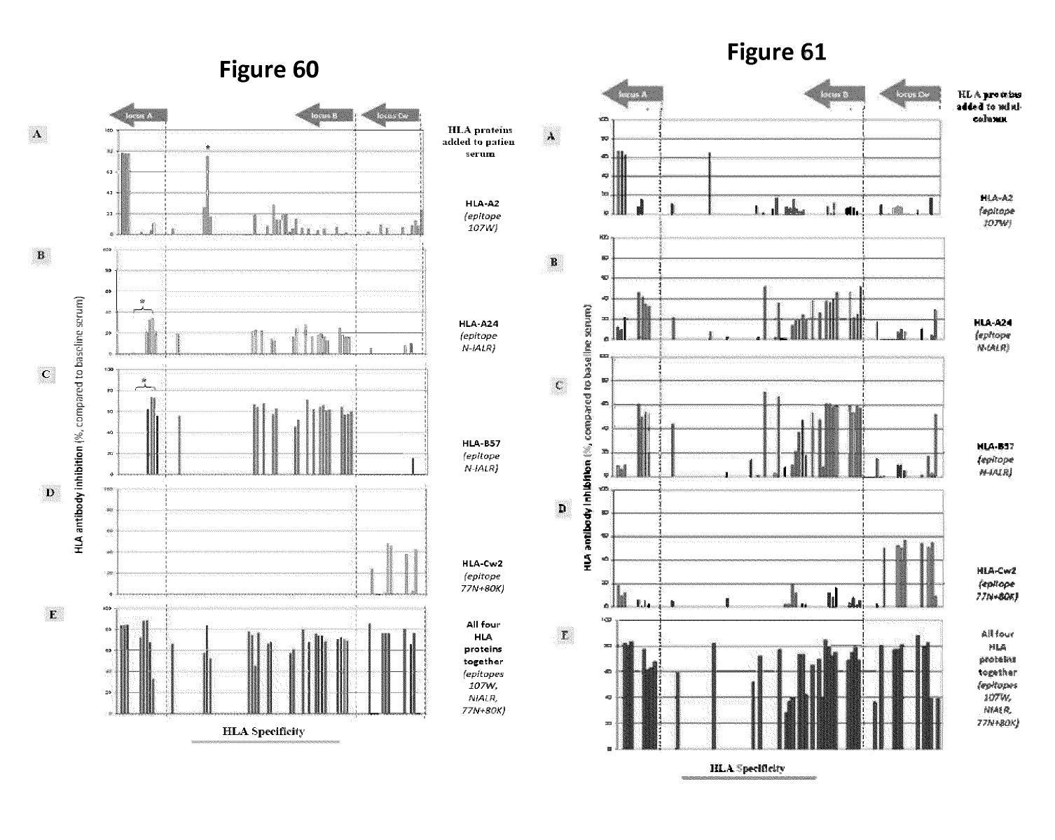

A second soluble phase analysis was carried out using a patient with a much more complex and diverse HLA reactive antibody spectrum. Patient 35 from the UHCW AIT program was selected. This patient had demonstrable alloantibody directed against HLA-A2, A69, Cw2, Cw4, Cw5, Cw6, Cw15, Cw17, and the public epitope HLA-Bw4. Epitope analysis of this profile suggested that the entire spectra of anti-HLA reactivity can be explained by reactivity against 3 individual epitopes: 107W (HLA-A2, A69), 84N-IALR (Bw4), and 77N+80K (Cw specificities). Soluble inhibition was performed using HLA-A2, A24 (for Bw4 expression), B57 (for Bw4 expression), and Cw2 and analyzed as before. Specific antibody reduction was seen for all four proteins (FIG. 60A-D), with a typical level of reduction in the range of 50-80%. A fresh serum aliquot was then incubated with a mixture of all four proteins simultaneously (final concentration of each protein 0.05 μg/μl). Effective inhibition of the patient's entire class I HLA reactive repertoire was observed, with a median antibody reduction for all specificities of 72.3% (FIG. 60E).

Antibody Removal using HLA Protein Columns: HLA specific antibody from patient 35 was then applied to HLA protein mini-columns. One ml of patient serum was applied to each of HLA-A2, A24, B57, and Cw2 200 mg protein columns and allowed to absorb via gravity flow. Once again, clear epitope specific removal was seen with each protein, with removal efficacy in the range of 50-80% (FIG. 61A-D). One hundred micrograms of each protein was then added to a fresh column to produce a 400 μg mini-column. Four ml of fresh patient serum was applied to this column, and removal of all epitope specificities was observed in complete concordance with the single protein mini-column data (FIG. 61E). The median antibody reduction across all HLA reactive specificities was 73.6%.

### Discussion of Example 7

Current protocols to reduce the levels of donor HLA-specific antibody prior to HLA incompatible transplantation have the major disadvantage of being non-specific, leading to a general comprising of overall humoral immunity. This Example describes the use of soluble HLA proteins and its ability to inhibit the anti-HLA response both in liquid and solid phase (mini-column) format. This Example has also demonstrated the isolation of HLA specific alloantibody from HLA protein columns and the characterization of their isotype composition and complement activating capability.

The soluble phase inhibition analysis is an effective means with which to define antibody specificity beyond the antigenic level and directly identify potential epitope specific reactivity. Knowledge of specific commonly reactive epitopes enables the design of a soluble phase absorption matrix which can be tailored to suit the individual patient profile. This is demonstrated clearly here with the selection of patients 35 and 65 from the antibody incompatible transplant cohort. For example, the entire class I HLA reactive antibody profile can be reduced by a single protein (HLA-B7) for patient 65 and by four proteins for patient 35 (HLA-A2, A24, B57, and Cw2). Patients with more complex antibody profiles may require an increased number of specific absorptions to elucidate the specific epitopes recognized by their HLA specific antibody fraction. These soluble phase studies using miniscule amounts of soluble HLA protein therefore provide strong support for an epitope specific approach to HLA specific antibody absorption.

HLA protein mini-columns were equally effective at removing HLA specific antibody in an epitope specific manner. Highly specific antibody reduction, typically 50-80%, in a single absorption was routinely observed. Once again, antibody specificities that did not express the epitope carried on the column protein were retrieved completely.

Thus, in accordance with the presently disclosed and claimed inventive concept(s), there have been provided anti-MHC removal devices, as well as methods of production and use thereof, that fully satisfy the objectives and advantages set forth hereinabove. Although the presently disclosed and claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the invention.

### REFERENCES

The following references, to the extent that they provide exemplary procedural or other details supplementary to those set forth herein, are specifically incorporated herein by reference.
Adams, S. 2000. Nat Med 6:337-342.
Amico, P et al. 2009 (Epub Sep. 11, 2009). Curr Opin Organ Transplant. 14(6):656-61.
Bohmig, G. A., et al. 2000. Am J Kidney Dis 35:667-673.
Busch, et al. 2002 (Epub May 16, 2002). J Immunol Methods. 263(1-2):111-21.
Cardella, C. J., et al., 197. Lancet, 1(8005): 264.
Chang, H., et al. 1994. Immunology, 91:11408-11412.
Chapman, J. R., et al., 1986. Transplantation, 42(1): 91-3.
Claas FH. 2010 (Epub Jul. 9, 2010). Curr Opin Organ Transplant. 15(4):462-6.
Claas FH. 2010. Current opinion in organ transplantation. 15(4):462-6.
Clatworthy, M R et al. 2010 (Epub Oct. 12, 2010). Curr Opin Immunol. 22(5):669-81.
Colvin RB. 2007. Journal of the American Society of Nephrology, 18(4):1046-56.
Crawford, F., et al. 1998. Immunity, 8:675-682.
Crew, R J et al. 2010 (Epub Jul. 9, 2010). Curr Opin Organ Transplant. 15(4):526-30.
Ditschkowski, M., et al. 1999. Ann Surg. 229(2): 246-254.
Einecke G, et al., 2009. American journal of transplantation, 9(11):2520-31.
El-Awar, N et al. 2007 (Epub Jul. 22, 2008). Clin Transpl. 175-94.
El-Awar, N., et al., 2007. Hum Immunol, 68(3): 170-80.
El-Awar, N. R., et al., 2007. Transplantation, 84(4): 532-40.
Emonds, M. P., et al. 2000. Pediatr Transplant, 4:6-11.
Gaseitsiwe and Maeurer, 2009. Methods in Mol. Bio. 524:417-26.
Gaston R S, et al., 2010. Transplantation, 90(1):68-74.
Gentry S E, et al., 2005. American journal of transplantation, 5(8):1914-21.
Gloor, J et al. 2008 (Epub May 31, 2008). Am J. Transplant. 8(7):1367-73.
Gloor, J. M., et al., 2004. Transplantation, 78(2): 221-7.
Gronski and Weinem, 2006. Rev. Diabet. Stud. 3:88-95.
Guerra, C. B., et al. 1998. J. of Immunol., 160: 4289-4297.
Hansen, John A. 2005. Biology of Blood and Marrow Transplantation 11:24-27.
Herold et al., 2009. Clin Immunol. 132:166-173.
Hickman, H., et al. 2003. J. of Immunology, 171: 22-26.
Higgins, R., et al., 2008. J Ren Care, 34(2): 85-93.
Higgins, R. M., et al., 1996. Nephron, 74(1): 53-7.
Howden, A. J., et al. 2000. Hum Immunol, 61:419-24.
Jindra P T, et al., 2008. J Immunol, 180(4):2357-66.
Jones et al., 2006. Nat. Rev. Immunol. 6:271-282.
Kalandadze, A et al. 1996 (Epub Aug. 16, 1996). J Biol. Chem. 271(33):20156-62.
Kalandadze, A., et al. 1996. J. Bio. Chem. 271:20156-20162.
Kaufman and Herold. 2009. Diabetes Metab. Res. Rev. 25:302-6.
Kezuka, T., et al., 2001. Arch Ophthalmol. 119(7):1044-9.
Lampson, L. and Levy, R. 1980. J. Immunol. 125: 293-299.
Landschulz, W., et al. 1988. Science, Vol. 240.
Mao, Q., et al., 2007. Am J Transplant, 7(4): 864-71.
Mao, Q., et al., 2007. Transplantation, 83(1): 54-61.
McMurtrey, C., et al. 2008. PNAS, 105:2981-2986.
Muller-Steinhardt, M., et al. 2000. Clin Transplant, 14:85-9.
Murata, K et al. 2007 (Epub Sep. 18, 2007). Am J. Transplant. 7(11):2605-14.
Nankivell, B J et al. 2010 (Epub Oct. 12, 2010). N Engl J. Med. 363(15):1451-62.
Narayanan K, et al. 2006. Transplant immunology, 15(3):187-97.
Nepom and Kwok, 1998. Diabetes, 47:1177-84.
Novak, E., et al. 1999. J. Clin. Invest. 104:R63-R67.
Palmer, A., et al., 1989. Lancet, 1(8628): 10-2.
Pratesi, F., et al. 2000. J Rheumatol, 27:109-15.
Prilliman, K. R., et al. 1999. J. Immunology, 162:7277.
Rahimi S, et al., 2004. American journal of transplantation, 4(3):326-34.
Rydberg, L., et al., 2005. Transpl Int, 17(11): 666-72.
Schmaldienst, S et al. 2001 (Epub May 24, 2001). Rheumatology (Oxford). 40(5):513-21.
Schuna, A. A. and C. Megeff. 2000. Am J Health Syst Pharm, 57:225-34. 2000.
Segev D L, et al., 2005. American journal of transplantation, 5(10):2448-55.
Starzl, T. E., et al., 1964. Surgery, 55: 195-200.
Streilein, J W et al. 2007. Ocul Immunol Inflamm. 15(3):187-94.
Takahashi, K. 2007 (Epub Jun. 27, 2007). Clin Exp Nephrol. 11(2):128-41.
Tanabe, K., 2007. Transplantation, 84(12 Suppl): S30-2.
Todd et al., 1988. Science, 240:1003-1009.
Tyden, G., et al., 2007. Transplantation, 83(9): 1153-5.
Warren, D S et al. 2010 (Epub Jan. 21, 2010). Immunol Res. 47(1-3):257-64.
Wasowska BA. 2010. Immunologic research, 47(1-3):25-44.
Wasowska, BA. 2010 (Epub Feb. 6, 2010). Immunol Res. 47(1-3):25-44.
Weber et al., 2007. Oncologist. 12:864-72.
Wettstein, D. A., et al. 1991. J. Exp. Med. 174:219-228.
Wicker et al., 1996. J. Clin. Invest. 98:2597-2603.
Williams, G. M., et al., 1968. N Engl J Med, 279(12): 611-8.
Wilson, B., 1981. Scand. J. Immunol., 14:201-205.
Yoon and Jun, 2001. Ann N Y Acad. Sci. 928:200-11.
Yoshida and Kikutani, 2000. Rev. Immunogenet. 2:140-6.
Zachary, A. A., et al., 2007. Tissue Antigens, 69 Suppl 1: 160-73.
Zimmer, K. P., et al. 1995. Gut. 36: 703-709.

What is claimed is:

1. An anti-MHC removal device, comprising:
a solid support;
a serologically active, soluble MHC moiety covalently coupled to the solid support and disposed on a surface of the anti-MHC removal device, the MHC moiety capable of interacting with a sample brought into contact with the surface of the device having the serologically active, soluble MHC moiety disposed thereon, whereby antibodies specific for the MHC moiety present in the sample will bind thereto, resulting in removal of said antibodies from the sample.

2. The anti-MHC removal device of claim 1, wherein the MHC moiety is further defined as a soluble class I HLA trimolecular complex produced by a method comprising the steps of:
cloning a nucleotide segment into a mammalian expression vector, the nucleotide segment encoding a desired individual class I MHC heavy chain that has the coding regions encoding the cytoplasmic and transmembrane domains of the desired individual class I MHC heavy chain allele removed such that the nucleotide segment encodes a truncated, soluble form of the desired individual class I MHC heavy chain molecule, thereby forming a construct that encodes the desired individual soluble class I MHC heavy chain molecule;
transfecting a mammalian cell line with the construct to provide a mammalian cell line expressing a construct that encodes a recombinant, individual soluble class I MHC heavy chain molecule, wherein the mammalian cell line is able to naturally process proteins into peptide ligands for loading into antigen binding grooves of MHC molecules, and wherein the mammalian cell line expresses beta-2-microglobulin;
culturing the mammalian cell line under conditions which allow for expression of the recombinant individual soluble class I MHC heavy chain molecule from the construct, such conditions also allowing for endogenous loading of a peptide ligand into the antigen binding groove of each recombinant, individual soluble class I MHC heavy chain molecule and non-covalent association of native, endogenously produced beta-2-microglobulin to form the individual soluble class I MHC trimolecular complexes prior to secretion of the individual soluble class I MHC trimolecular complexes from the cell;
harvesting the soluble class I MHC trimolecular complexes from the culture while retaining the mammalian cell line in culture for production of additional soluble class I MHC trimolecular complexes; and
purifying the individual, soluble class I MHC trimolecular complexes substantially away from other proteins, wherein the individual soluble class I MHC trimolecular complexes maintain the physical, functional and antigenic integrity of the native class I MHC trimolecular complex, and wherein each trimolecular complex so purified comprises identical recombinant, individual soluble class I MHC heavy chain molecules.

3. The anti-MHC removal device of claim 1, wherein the MHC moiety is further defined as a soluble class II HLA trimolecular complex produced by a method comprising the steps of:
inserting a first isolated nucleic acid segment and a second isolated nucleic acid segment into a mammalian cell line, the first isolated nucleic acid segment encoding a soluble form of an alpha chain of a HLA class II molecule having a first domain of a super secondary structural motif attached thereto, and the second isolated nucleic acid segment encoding a soluble form of a beta chain of the HLA class II molecule having a second domain of the super secondary structural motif attached thereto, wherein the mammalian cell line is a non-human mammalian cell line or a human cell line that does not express endogenous HLA class II, and wherein the mammalian cell line comprises glycosylation mechanisms required for glycosylation of proteins produced therein and chaperone complexes required for peptide ligand loading into HLA class II molecules;
culturing the recombinant mammalian cell line under conditions that allow for expression of the soluble class II alpha and beta chains, association of the soluble class II alpha and beta chains through the first and second domains of the super secondary structural motif, glycosylation of the soluble class II alpha and beta chains, and loading of an antigen binding groove formed from the soluble class II alpha and beta chains with an endogenously produced, non-covalently associated peptide ligand, thereby producing soluble class II trimolecular complexes;
isolating the soluble class II trimolecular complexes secreted from the recombinant mammalian cell line; and
purifying the soluble class II trimolecular complexes substantially away from other proteins.

4. The anti-MHC removal device of claim 1, wherein the solid support is selected from the group consisting of a well, a bead, a membrane, a microtiter plate, a matrix, a pore, plastic, glass, a polymer, a polysaccharide, nylon, nitrocellulose, a paramagnetic compound, and combinations thereof.

5. The anti-MHC removal device of claim 4, wherein the solid support is further defined as an N-hydroxysuccinimide (NHS)-activated SEPHAROSE® matrix.

6. The anti-MHC removal device of claim 1, wherein the soluble MHC moiety is coupled to the solid support via a covalent amide bond formed between a primary amino group contained within the HLA moiety and an ester group contained in the solid support.

7. The anti-MHC removal device of claim 1, wherein the solid support further comprises a spacer arm.

8. The anti-MHC removal device of claim 1, further defined as a human use device.

9. The anti-MHC removal device of claim 8, further defined as an extracorporeal plasmapheresis human use device.

10. A kit containing the anti-MHC removal device of claim 1.

11. The kit of claim 10, further comprising at least one reagent for elution of antibodies from the anti-MHC removal device.

12. A method of removing anti-HLA antibodies from a biological sample, the method comprising the steps of:
(a) contacting a biological sample with the anti-MHC removal device of claim 1, whereby antibodies specific for the MHC moiety present on a surface of the anti-MHC removal device are removed from the biological sample; and
(b) recovering the biological sample, whereby the antibodies specific for the MHC moiety are substantially reduced in the recovered biological sample.

13. The method of claim 12, wherein the biological sample is selected from the group consisting of serum, tissue, blood, plasma, cerebrospinal fluid, tears, saliva, lymph, dialysis fluid, organ or tissue culture derived fluids, fluids extracted from physiological tissues, and combinations thereof.

14. The method of claim 12, further comprising repeating steps (a) and (b).

15. The method of claim 12, further comprising the step of eluting antibodies from the anti-MHC removal device.

16. The method of claim 12, wherein the MHC moiety is a class I MHC trimolecular complex.

17. The method of claim 12, wherein the MHC moiety is a class II MHC trimolecular complex.

18. The method of claim 12, wherein the solid support of the anti-MHC removal device is selected from the group consisting of a well, a bead, a membrane, a microtiter plate, a matrix, a pore, plastic, glass, a polymer, a polysaccharide, nylon, nitrocellulose, a paramagnetic compound, and combinations thereof.

19. The method of claim 18, wherein the solid support is further defined as an N-hydroxysuccinimide (NHS)-activated SEPHAROSE® matrix.

20. The method of claim 12, further comprising the step of placing the recovered biological sample back into the patient.


SEQUENCE LISTING (9 sequences)

SEQ ID NO: 1 - DNA, 786 bp, Artificial Sequence
  construct encoding truncated, soluble human DRA1*0101 fused to linker and leucine zipper motif
atggccataagtggagtccctgtgctaggatttttcatcatagctgtgctgatgagcgctcaggaatcatgggctdraatcaaagaagaacatgtgatcatccaggccgagttctatctgaatcctgaccaatcaggcgagtttatgtttgacdratttgatggtgatgagattttccatgtggatatggcaaagaaggagacggtctggcggcttgaagaatttggacgadratttgccagctttgaggctcaaggtgcattggccaacatagctgtggacaaagccaacctggaaatcatgacaaagdracgctccaactatactccgatcaccaatgtacctccagaggtaactgtgctcacgaacagccctgtggaactgagadragagcccaacgtcctcatctgtttcatcgacaagttcaccccaccagtggtcaatgtcacgtggcttcgaaatggadraaaacctgtcaccacaggagtgtcagagacagtcttcctgcccagggaagaccaccttttccgcaagttccactatdractccccttcctgccctcaactgaggacgtttacgactgcagggtggagcactggggcttggatgagcctcttctcdraaagcactgggagtttgatgctccaagccctctcccagagactacagaggtcgacggaggaggaggtggagctcagctcgaaaaagagctccaggccctggagaaggaaaatgcacagctggaatgggagttgcaagcactggaaaaggaactggctcagtga

SEQ ID NO: 2 - protein, 253 aa, Artificial Sequence
  amino acid sequence encoded by SEQ ID NO1
MAISGVPVLGFFIIAVLMSAQESWAIKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGAQLEKELQALEKENAQLEWELQALEKELAQ

SEQ ID NO: 3 - DNA, 822 bp, Artificial Sequence
  construct encoding truncated, soluble human DRB1*040101 fused to a linker and leucine zipper motif
atggtgtgtctgaagttccctggaggctcctgcatggcagctctgacagtgacactgatggtgctgagctccccadrbctggctttggctggggacacccgaccacgtttcttggagcaggttaaacatgagtgtcatttcttcaacgggacgdrbgagcgggtgcggttcctggacagatacttctatcaccaagaggagtacgtgcgcttcgacagcgacgtgggggagdrbtaccgggcggtgacggagctggggcggcctgatgccgagtactggaacagccagaaggacctcctggagcagaagdrbcgggccgcggtggacacctactgcagacacaactacggggttggtgagagcttcacagtgcagcggcgagtctatdrbcctgaggtgactgtgtatcctgcaaagacccagcccctgcagcaccacaacctcctggtctgctctgtgaatggtdrbttctatccaggcagcattgaagtcaggtggttccggaacggccaggaagagaagactggggtggtgtccacaggcdrbctgatccagaatggagactggaccttccagaccctggtgatgctggaaacagttcctcggagtggagaggtttacdrbacctgccaagtggagcacccaagcctgacgagccctctcacagtggaatggagagcacggtctgaatctgcacagdrbagcaaggtcgacggaggaggaggtggagctcagttgaaaaagaaattgcaagcactgaagaaaaagaacgctcagctgaagtggaaacttcaagccctcaagaagaaactcgcccagtga

SEQ ID NO: 4 - protein, 264 aa, Artificial Sequence
  amino acid sequence encoded by SEQ ID NO3
MVCLKFPGGSCMAALTVTLMVLSSPLALAGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARSESAQSKVDGGGGGAQLKKKLQALKKKNAQLKWKLQALKKKLAQ

SEQ ID NO: 5 - DNA, 825 bp, Artificial Sequence
  construct encoding truncated, soluble human DRB1*0103 fused to a linker and leucine zipper motif
atggtgtgtctgaagctccctggaggctcctgcatgacagcgctgacagtgacactgatggtgctgagctccccadrbctggctttggctggggacacccgaccacgtttcttgtggcagcttaagtttgaatgtcatttcttcaatgggacgdrbgagcgggtgcggttgctggaaagatgcatctataaccaagaggagtccgtgcgcttcgacagcgacgtgggggagdrbtaccgggcggtgacggagctggggcggcctgatgccgagtactggaacagccagaaggacatcctggaagacgagdrbcgggccgcggtggacacctactgcagacacaactacggggttggtgagagcttcacagtgcagcggcgagttgagdrbcctaaggtgactgtgtatccttcaaagacccagcccctgcagcaccacaacctcctggtctgctctgtgagtggtdrbttctatccaggcagcattgaagtcaggtggttccggaacggccaggaagagaaggctggggtggtgtccacaggcdrbctgatccagaatggagattggaccttccagaccctggtgatgctggaaacagttcctcggagtggagaggtttacdrbacctgccaagtggagcacccaagtgtgacgagccctctcacagtggaatggagagcacggtctgaatctgcacagdrbagcaaggtcgacggaggaggaggtggagctcagttgaaaaagaaattgcaagcactgaagaaaaagaacgctcagdrbctgaagtggaaacttcaagccctcaagaagaaactcgcccagtga

SEQ ID NO: 6 - protein, 264 aa, Artificial Sequence
  amino acid sequence encoded by SEQ ID NO5
MVCLKLPGGSCMTALTVTLMVLSSPLALAGDTRPRFLWQLKFECHFFNGTERVRLLERCIYNQEESVRFDSDVGEYRAVTELGRPDAEYWNSQKDILEDERAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRARSESAQSKVDGGGGGAQLKKKLQALKKKNAQLKWKLQALKKKLAQ

SEQ ID NO: 7 - protein, 7 aa, Artificial Sequence
  amino acid linker sequence
DVGGGGG

SEQ ID NO: 8 - protein, 228 aa, Artificial Sequence
  sHLA-DRA1*01 01 ACIDp1
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDAPSPLPETTEVDGGGGGAQLEKELQALEKENAQLEWELQALEKELAQ

SEQ ID NO: 9 - protein, 235 aa, Artificial Sequence
  sHLA-DRB1*1101 BASEp1
GDTRPRFLEYSTSECHFFNGTERVRFLDRYFYNQEEYVRFDSDVGEFRAVTELGRPDEEYWNSQKDFLEDRRAAVDTYCRHNYGVGESFTVQRRVHPKVTVYPSKTQPLQHHNLLVCSVSGFYPGSIEVRWFRNGQEEKTGVVSTGLIHNGDWTFQTLVMLETVPRSGEVYTCQVEHPSVTSPLTVEWRARSESAQSKVDGGGGGAQLKKKLQALKKKNAQLKWKLQALKKKLAQ